(19) United States
(12) Patent Application Publication
(10) Pub. No.: US 2010/0311701 A1
(43) Pub. Date: Dec. 9, 2010

(54) Pharmaceutical Co-Crystal Compositions

(75) Inventors: Orn Almarsson, Shrewsbury, MA (US); Magali Bourghol Hickey, Medford, MA (US); Matthew Peterson, Hopkinton, MA (US); Michael J. Zaworotko, Tampa, FL (US); Brian Moulton, Providence, RI (US); Nair Rodriguez-Hornedo, Ann Arbor, MI (US)

Correspondence Address: SALIWANCHIK LLOYD & SALIWANCHIK;A PROFESSIONAL ASSOCIATION, PO Box 142950, GAINESVILLE, FL, 32614, US

(73) Assignees: Transform Pharmaceuticals, Inc, Lexington, MA (US); University Of South Florida, Tamp, FL (US); The Regents of the University Of Michigan, Ann Arbor, MI (US)

(21) Appl. No.: 12/792,415

(22) Filed: Jun. 2, 2010

Related U.S. Application Data
(63) Continuation of application No. 10/546,963, filed on Aug. 26, 2005, now abandoned.
Application No. 10/546,963 is a continuation-in-part of application No. 10/660,202, filed on Sep. 11, 2003.
Application No. PCT/US04/06288 is a continuation-in-part of application No. PCT/US03/27772, filed on Sep. 4, 2003.
Application No. PCT/US03/27772 is a continuation-in-part of application No. 10/378,956, filed on Mar. 3, 2003.
Application No. 10/660,202 is a continuation-in-part of application No. 10/637,829, filed on Aug. 8, 2003, now abandoned.
Application No. 10/637,829 is a division of application No. 10/295,995, filed on Nov. 18, 2002, now Pat. No. 6,699,840.
Application No. 10/295,995 is a continuation of application No. 10/232,589, filed on Sep. 3, 2002, now Pat. No. 6,559,293.
Application No. PCT/US03/27772 is a continuation-in-part of application No. 10/637,829, filed on Aug. 8, 2003, now abandoned.
Application No. 10/637,829 is a division of application No. 10/295,995, filed on Nov. 18, 2002, now Pat. No. 6,699,840.
Application No. 10/295,995 is a continuation of application No. 10/232,589, filed on Sep. 3, 2002, now Pat. No. 6,559,293.
Application No. 10/660,202 is a continuation-in-part of application No. 10/449,307, filed on May 30, 2003, now Pat. No. 7,078,526.
Application No. PCT/US03/27772 is a continuation-in-part of application No. 10/449,307, filed on May 30, 2003, now Pat. No. 7,078,526.
Application No. 10/660,202 is a continuation-in-part of application No. 10/601,092, filed on Jun. 20, 2003, now abandoned.
Application No. PCT/US03/27772 is a continuation-in-part of application No. 10/601,092, filed on Jun. 20, 2003, now abandoned.
Application No. PCT/US04/06288 is a continuation-in-part of application No. PCT/US03/06662, filed on Mar. 3, 2003.
Application No. PCT/US04/06288 is a continuation-in-part of application No. 10/637,829, filed on Aug. 8, 2003, now abandoned.
Application No. 10/637,829 is a division of application No. 10/295,995, filed on Nov. 18, 2002, now Pat. No. 6,699,840.
Application No. 10/295,995 is a continuation of application No. 10/232,589, filed on Sep. 3, 2002, now Pat. No. 6,559,293.
Application No. PCT/US04/06288 is a continuation-in-part of application No. 10/449,307, filed on May 30, 2003, now Pat. No. 7,078,526.
Application No. PCT/US04/06288 is a continuation-in-part of application No. 10/601,092, filed on Jun. 20, 2003, now abandoned.
Application No. 10/601,092 is a continuation-in-part of application No. PCT/US03/41273, filed on Dec. 24, 2003.
Application No. PCT/US03/41273 is a continuation-in-part of application No. PCT/US03/19574, filed on Jun. 20, 2003.
(60) Provisional application No. 60/451,213, filed on Feb. 28, 2003.
(60) Provisional application No. 60/463,962, filed on Apr. 18, 2003.
(60) Provisional application No. 60/487,064, filed on Jul. 11, 2003.
(60) Provisional application No. 60/360,768, filed on Mar. 1, 2002.
(60) Provisional application No. 60/451,213, filed on Feb. 28, 2003.
(60) Provisional application No. 60/463,962, filed on Apr. 18, 2003.
(60) Provisional application No. 60/487,064, filed on Jul. 11, 2003.
(60) Provisional application No. 60/406,974, filed on Aug. 30, 2002.
(60) Provisional application No. 60/380,288, filed on May 15, 2002.
(60) Provisional application No. 60/356,764, filed on Feb. 15, 2002.
(60) Provisional application No. 60/356,764, filed on Feb. 15, 2002.
(60) Provisional application No. 60/380,288, filed on May 15, 2002.
(60) Provisional application No. 60/406,974, filed on Aug. 30, 2002.
(60) Provisional application No. 60/463,962, filed on Apr. 18, 2003.
(60) Provisional application No. 60/444,315, filed on Jan. 31, 2003.
(60) Provisional application No. 60/439,282, filed on Jan. 10, 2003.
(60) Provisional application No. 60/384,152, filed on May 31, 2002.
(60) Provisional application No. 60/463,962, filed on Apr. 18, 2003.
(60) Provisional application No. 60/444,315, filed on Jan. 31, 2003.
(60) Provisional application No. 60/439,282, filed on Jan. 10, 2003.
(60) Provisional application No. 60/384,152, filed on May 31, 2002.
(60) Provisional application No. 60/451,213, filed on Feb. 28, 2003.
(60) Provisional application No. 60/451,213, filed on Feb. 28, 2003.
(60) Provisional application No. 60/360,768, filed on Mar. 1, 2002.
(60) Provisional application No. 60/406,974, filed on Aug. 30, 2002.
(60) Provisional application No. 60/380,288, filed on May 15, 2002.
(60) Provisional application No. 60/356,764, filed on Feb. 15, 2002.
(60) Provisional application No. 60/463,962, filed on Apr. 18, 2003.
(60) Provisional application No. 60/444,315, filed on Jan. 31, 2003.
(60) Provisional application No. 60/439,282, filed on Jan. 10, 2003.
(60) Provisional application No. 60/384,152, filed on May 31, 2002.
(60) Provisional application No. 60/451,213, filed on Feb. 28, 2003.
(60) Provisional application No. 60/508,208, filed on Oct. 2, 2003.
(60) Provisional application No. 60/542,752, filed on Feb. 6, 2004.
(60) Provisional application No. 60/390,881, filed on Jun. 21, 2002.
(60) Provisional application No. 60/426,275, filed on Nov. 14, 2002.
(60) Provisional application No. 60/427,086, filed on Nov. 15, 2002.
(60) Provisional application No. 60/429,515, filed on Nov. 26, 2002.
(60) Provisional application No. 60/437,516, filed on Dec. 30, 2002.
(60) Provisional application No. 60/456,027, filed on Mar. 18, 2003.

(51) Int. Cl. A61K 31/616; A61K 31/415; A61K 31/455; A61K 31/335; A61K 31/357; A61K 31/5513; A61K 31/4196; A61K 31/194; A61K 31/165; A61K 31/191; A61K 31/513; A61K 31/17; A61K 31/549; A61K 31/167; A61K 31/444; A61K 31/4166; A61K 31/4412; A61K 31/192; A61K 31/55; A61K 31/12

(52) U.S. Cl. 514/165; 514/406; 514/355; 514/450; 514/454; 514/220; 514/383; 514/574; 514/618; 514/557; 514/274; 514/588; 514/223.2; 514/223.5; 514/356; 514/629; 514/334; 514/391; 514/345; 514/570; 514/217; 514/688; 514/568

(57) ABSTRACT

A pharmaceutical composition comprising a co-crystal of an API and a co-crystal former; wherein the API has at least one functional group selected from ether, thioether, alcohol, thiol, aldehyde, ketone, thioketone, nitrate ester, phosphate ester, thiophosphate ester, ester, thioester, sulfate ester, carboxylic acid, phosphonic acid, phosphinic acid, sulfonic acid, amide, primary amine, secondary amine, ammonia, tertiary amine, imine, thiocyanate, cyanamide, oxime, nitrile diazo, organohalide, nitro, S-heterocyclic ring, thiophene, N-heterocyclic ring, pyrrole, O-heterocyclic ring, furan, epoxide, peroxide, hydroxamic acid, imidazole, pyridine and the co-crystal former has at least one functional group selected from amine, amide, pyridine, imidazole, indole, pyrrolidine, carbonyl, carboxyl, hydroxyl, phenol, sulfone, sulfonyl, mercapto and methyl thio, such that the API and co-crystal former are capable of co-crystallizing from a solution phase under crystallization conditions.

### CROSS-REFERENCE TO RELATED APPLICATIONS

This application is a continuation of U.S. patent application Ser. No. 10/546,963, filed Aug. 26, 2005, which is the U.S. national stage application of International Patent Application No. PCT/US2004/006288, filed Feb. 26, 2004 each of which are hereby incorporated herein by reference in its entirety for all purposes.

PCT/US2004/006288 is a continuation-in-part of U.S. patent application Ser. No. 10/660,202, filed Sep. 11, 2003, which claims the benefit of U.S. Provisional Patent Application No. 60/451,213, filed Feb. 28, 2003; U.S. Provisional Patent Application No. 60/463,962, filed Apr. 18, 2003; and U.S. Provisional Application No. 60/487,064, filed Jul. 11, 2003 each of which are hereby incorporated herein by reference in its entirety for all purposes.

PCT/US2004/006288 is also a continuation-in-part of PCT/US03/27772, filed on Sep. 4, 2003 which is a continuation-in-part of U.S. patent application Ser. No. 10/378,956, filed Mar. 1, 2003, which claims the benefit of U.S. Provisional Application No. 60/360,768, filed Mar. 1, 2002; said PCT/US03/27772 also claims the benefit of U.S. Provisional Patent Application No. 60/451,213, filed Feb. 28, 2003; U.S. Provisional Patent Application No. 60/463,962, filed Apr. 18, 2003; and U.S. Provisional Application No. 60/487,064, filed Jul. 11, 2003 each of which are hereby incorporated by reference in its entirety for all purposes.

Said Ser. No. 10/660,202 and PCT/US03/27772 are also continuations-in-part of U.S. patent application Ser. No. 10/637,829, filed Aug. 8, 2003, which is a divisional of U.S. patent application Ser. No. 10/295,995, filed Nov. 18, 2002, which is a continuation of U.S. patent application Ser. No. 10/232,589, filed Sep. 3, 2002, which claims the benefit of U.S. Provisional Patent Application No. 60/406,974, filed Aug. 30, 2002 and U.S. Provisional Patent Application No. 60/380,288, filed May 15, 2002 and U.S. Provisional Patent Application No. 60/356,764, filed Feb. 15, 2002 each of which are hereby incorporated by reference in its entirety for all purposes.

Said Ser. No. 10/660,202 and PCT/US03/27772 are also continuations-in-part of U.S. patent application Ser. No. 10/449,307, filed May 30, 2003 which claims the benefit of U.S. Provisional Patent Application No. 60/463,962, filed Apr. 18, 2003 and U.S. Provisional Patent Application No. 60/444,315, filed Jan. 31, 2003 and U.S. Provisional Patent Application No. 60/439,282, filed Jan. 10, 2003 and U.S. Provisional Patent Application No. 60/384,152, filed May 31, 2002 each of which are hereby incorporated by reference in its entirety for all purposes.

Said Ser. No. 10/660,202 and PCT/US03/27772 are also continuations-in-part of U.S. patent application Ser. No. 10/601,092, filed Jun. 20, 2003, which claims the benefit of U.S. Provisional Patent Application No. 60/451,213, filed Feb. 28, 2003 each of which are hereby incorporated by reference in its entirety for all purposes.

PCT/US2004/006288 is also a continuation-in-part of PCT/US03/06662, filed Mar. 3, 2003, which claims the benefit of U.S. Provisional Application No. 60/360,768, filed Mar. 1, 2002.

PCT/US2004/006288 is also a continuation-in-part of U.S. patent application Ser. No. 10/637,829, filed Aug. 8, 2003, which is a divisional of U.S. patent application Ser. No. 10/295,995, filed Nov. 18, 2002, which is a continuation of U.S. patent application Ser. No. 10/232,589, filed Sep. 3, 2002, which claims the benefit of U.S. Provisional Patent Application No. 60/406,974, filed Aug. 30, 2002 and U.S. Provisional Patent Application No. 60/380,288, filed May 15, 2002 and U.S. Provisional Patent Application No. 60/356,764, filed Feb. 15, 2002 each of which are hereby incorporated by reference in its entirety for all purposes.

PCT/US2004/006288 is also a continuation-in-part of U.S. patent application Ser. No. 10/449,307, filed May 30, 2003, which claims the benefit of U.S. Provisional Patent Application No. 60/463,962, filed Apr. 18, 2003 and U.S. Provisional Patent Application No. 60/444,315, filed Jan. 31, 2003 and U.S. Provisional Patent Application No. 60/439,282, filed Jan. 10, 2003 and U.S. Provisional Patent Application No. 60/384,152, filed May 31, 2002 each of which are hereby incorporated by reference in its entirety for all purposes.

PCT/US2004/06288 is also a continuation-in-part of U.S. patent application Ser. No. 10/601,092, filed Jun. 20, 2003, which claims the benefit of U.S. Provisional Patent Application No. 60/451,213, filed Feb. 28, 2003 each of which are hereby incorporated by reference in its entirety for all purposes.

PCT/US2004/06288 claims the benefit of U.S. Provisional Patent Application No. 60/508,208, filed Oct. 2, 2003 and U.S. Provisional Patent Application No. 60/542,752, filed Feb. 6, 2004 each of which are hereby incorporated by reference in its entirety for all purposes.

PCT/US2004/06288 is also a continuation-in-part of PCT/US03/41273, filed Dec. 24, 2003, which is a continuation-in-part of PCT/US03/19574, filed Jun. 20, 2003, which claims the benefit of U.S. Provisional Application No. 60/390,881, filed Jun. 21, 2002, U.S. Provisional Application No. 60/426,275, filed Nov. 14, 2002; U.S. Provisional Application No. 60/427,086, filed Nov. 15, 2002; U.S. Provisional Application No. 60/429,515, filed Nov. 26, 2002; U.S. Provisional Application No. 60/437,516, filed Dec. 30, 2002; and U.S. Provisional Application No. 60/456,027, filed Mar. 18, 2003 each which are hereby incorporated by reference in its entirety for all purposes.

PCT/US2004/006288 is also a continuation-in-part of U.S. patent application Ser. No. 10/601,092, filed Jun. 20, 2003 which claims the benefit of U.S. Provisional Application No. 60/390,881, filed Jun. 21, 2002, U.S. Provisional Application No. 60/426,275, filed Nov. 14, 2002; U.S. Provisional Application No. 60/427,086, filed Nov. 15, 2002; U.S. Provisional Application No. 60/429,515, filed Nov. 26, 2002; U.S. Provisional Application No. 60/437,516, filed Dec. 30, 2002; and U.S. Provisional Application No.; and U.S. Provisional Application No. 60/456,027 filed on Mar. 18, 2003 each of which are hereby incorporated by reference in its entirety for all purposes.

### FIELD OF THE INVENTION

The present invention relates to co-crystal API-containing compositions, pharmaceutical compositions comprising such. APIs, and methods for preparing the same.

### BACKGROUND OF THE INVENTION

Active pharmaceutical ingredients (API or APIs (plural)) in pharmaceutical compositions can be prepared in a variety of different forms. Such APIs can be prepared so as to have a variety of different chemical forms including chemical derivatives or salts. Such APIs can also be prepared to have different physical forms. For example, the APIs may be amorphous, may have different crystalline polymorphs, or may exist in different solvation or hydration states. By varying the form of an API, it is possible to vary the physical properties thereof. For example, crystalline polymorphs typically have different solubilities from one another, such that a more thermodynamically stable polymorph is less soluble than a less thermodynamically stable polymorph. Pharmaceutical polymorphs can also differ in properties such as shelf-life, bioavailability, morphology, vapour pressure, density, colour, and compressibility. Accordingly, variation of the crystalline state of an API is one of many ways in which to modulate the physical properties thereof.

It would be advantageous to have new forms of these APIs that have improved properties, in particular, as oral formulations. Specifically, it is desirable to identify improved forms of APIs that exhibit significantly improved properties including increased aqueous solubility and stability. Further, it is desirable to improve the processability, or preparation of pharmaceutical formulations. For example, needle-like crystal forms or habits of APIs can cause aggregation, even in compositions where the API is mixed with other substances, such that a non-uniform mixture is obtained. It is also desirable to increase or decrease the dissolution rate of API-containing pharmaceutical compositions in water, increase or decrease the bioavailability of orally-administered compositions, and provide a more rapid or more delayed onset to therapeutic effect. It is also desirable to have a form of the API which, when administered to a subject, reaches a peak plasma level faster or slower, has a longer lasting therapeutic plasma concentration, and higher or lower overall exposure when compared to equivalent amounts of the API in its presently-known form. The improved properties discussed above can be altered in a way which is most beneficial to a specific API for a specific therapeutic effect.

### SUMMARY OF THE INVENTION

It has now been found that new co-crystalline forms of APIs can be obtained which improve the properties of APIs as compared to such APIs in a non-co-crystalline state (free acid, free base, zwitter ions, salts, etc.).

Accordingly, in a first aspect, the present invention provides a co-crystal pharmaceutical composition comprising an API compound and a co-crystal former, such that the API and co-crystal former are capable of co-crystallizing from a solid or solution phase under crystallization conditions.

Another aspect of the present invention provides a process for the production of a pharmaceutical composition, which process comprises:

(1) providing an API which has at least one functional group selected from ether, thioether, alcohol, thiol, aldehyde, ketone, thioketone, nitrate ester, phosphate ester, thiophosphate ester, ester, thioester, sulfate ester, carboxylic acid, phosphonic acid, phosphinic acid, sulfonic acid, amide, primary amine, secondary amine, ammonia, tertiary amine, imine, thiocyanate, cyanamide, oxime, nitrile, diazo, organohalide, nitro, S-heterocyclic ring, thiophene, N-heterocyclic ring, pyrrole, O-heterocyclic ring, furan, epoxide, peroxide, hydroxamic acid, imidazole, and pyridine;

(2) providing a co-crystal former which has at least one functional group selected from ether, thioether, alcohol, thiol, aldehyde, ketone, thioketone, nitrate ester, phosphate ester, thiophosphate ester, ester, thioester, sulfate ester, carboxylic acid, phosphonic acid, phosphinic acid, sulfonic acid, amide, primary amine, secondary amine, ammonia, tertiary amine, imine, thiocyanate, cyanamide, oxime, nitrile, diazo, organohalide, nitro, S-heterocyclic ring, thiophene, N-heterocyclic ring, pyrrole, O-heterocyclic ring, furan, epoxide, peroxide, hydroxamic acid, imidazole, and pyridine;

(3) grinding, heating, co-subliming, co-melting, or contacting in solution the API with the co-crystal former under crystallization conditions;

(4) isolating co-crystals formed thereby; and

(5) incorporating the co-crystals into a pharmaceutical composition.

A further aspect of the present invention provides a process for the production of a pharmaceutical composition, which comprises:

(1) grinding, heating, co-subliming, co-melting, or contacting in solution an API compound with a co-crystal former, under crystallization conditions, so as to form a solid phase;

(2) isolating co-crystals comprising the API and the co-crystal former; and

(3) incorporating the co-crystals into a pharmaceutical composition.

In a further aspect, the present invention provides a process for the production of a pharmaceutical composition, which comprises:

(1) providing (i) an API or a plurality of different APIs, and (ii) a co-crystal former or a plurality of different co-crystal formers, wherein at least one of the APIs and the co-crystal formers is provided as a plurality thereof;

(2) isolating co-crystals comprising the API and the co-crystal former; and

(3) incorporating the co-crystals into a pharmaceutical composition.

### Solubility Modulation

In a further aspect, the present invention provides a process for modulating the solubility of an API, which process comprises:

(1) grinding, heating, co-subliming, co-melting, or contacting in solution the API with a co-crystal former under crystallization conditions, so as to form a co-crystal of the API and the co-crystal former; and

(2) isolating co-crystals comprising the API and the co-crystal former.

### Dissolution Modulation

In a further aspect, the present invention provides a process for modulating the dissolution of an API, whereby the aqueous dissolution rate or the dissolution rate in simulated gastric fluid or in simulated intestinal fluid, or in a solvent or plurality of solvents is increased or decreased, which process comprises:

(1) grinding, heating, co-subliming, co-melting, or contacting in solution the API with a co-crystal former under crystallization conditions, so as to form a co-crystal of the API and the co-crystal former; and

(2) isolating co-crystals comprising the API and the co-crystal former.

In one embodiment, the dissolution of the API is increased.

### Bioavailability Modulation

In a further aspect, the present invention provides a process for modulating the bioavailability of an API, whereby the AUC is increased, the time to Tₘₐₓ is reduced, the length of time the concentration of the API is above ½ Tₘₐₓ is increased, or Cₘₐₓ is increased, which process comprises:

(1) grinding, heating, co-subliming, co-melting, or contacting in solution the API with a co-crystal former under crystallization conditions, so as to form a co-crystal of the API and the co-crystal former; and

(2) isolating co-crystals comprising the API and the co-crystal former.

### Dose Response Modulation

In a further aspect the present invention provides a process for improving the linearity of a dose response of an API, which process comprises:

(1) grinding, heating, co-subliming, co-melting, or contacting in solution an API with a co-crystal former under crystallization conditions, so as to form a co-crystal of the API and the co-crystal former; and

(2) isolating co-crystals comprising the API and the co-crystal former.

### Increased Stability

In a still further aspect the present invention provides a process for improving the stability of a pharmaceutical salt, which process comprises:

(1) grinding, heating, co-subliming, co-melting, or contacting in solution the pharmaceutical salt with a co-crystal former under crystallization conditions, so as to form a co-crystal of the API and the co-crystal former; and

(2) isolating co-crystals comprising the API and the co-crystal former.

### Difficult to Salt or Unsaltable Compounds

In a still further aspect the present invention provides a process for making co-crystals of difficult to salt or unsaltable APIs, which process comprises:

(1) grinding, heating, co-subliming, co-melting, or contacting in solution the API with a co-crystal former under crystallization conditions, so as to form a co-crystal of the API and the co-crystal former; and

(2) isolating co-crystals comprising the API and the co-crystal former.

### Decreasing Hygroscopicity

In a still further aspect the present invention provides a method for decreasing the hygroscopicity of an API, which method comprises:

(1) grinding, heating, co-subliming, co-melting, or contacting in solution the API with a co-crystal former under crystallization conditions, so as to form a co-crystal of the API and the co-crystal former; and

(2) isolating co-crystals comprising the API and the co-crystal former.

### Crystallizing Amorphous Compounds

In a still further embodiment aspect the present invention provides a process for crystallizing an amorphous compound, which process comprises:

(1) grinding, heating, co-subliming, co-melting, or contacting in solution the API with a co-crystal former under crystallization conditions, so as to form a co-crystal of the API and the co-crystal former; and

(2) isolating co-crystals comprising the API and the co-crystal former.

### Decreasing Form Diversity

In a still further embodiment aspect the present invention provides a process for reducing the form diversity of an API, which process comprises:

(1) grinding, heating, co-subliming, co-melting, or contacting in solution the API with a co-crystal former under crystallization conditions, so as to form a co-crystal of the API and the co-crystal former; and

(2) isolating co-crystals comprising the API and the co-crystal former.

### Morphology Modulation

In a still further embodiment aspect the present invention provides a process for modifying the morphology of an API, which process comprises:

(1) grinding, heating, co-subliming, co-melting, or contacting in solution the API with a co-crystal former under crystallization conditions, so as to form a co-crystal of the API and the co-crystal former; and

(2) isolating co-crystals comprising the API and the co-crystal former.

In a further aspect, the present invention provides a co-crystal composition comprising a co-crystal, wherein said co-crystal comprises an API compound and a co-crystal former. In further embodiments the co-crystal has an improved property as compared to the free form (including a free acid, free base, zwitter ion, hydrate, solvate, etc.) or a salt (which includes salt hydrates and solvates). In further embodiments, the improved property is selected from the group consisting of: increased solubility, increased dissolution, increased bioavailability, increased dose response, decreased hygroscopicity, a crystalline form of a normally amorphous compound, a crystalline form of a difficult to salt or unsaltable compound, decreased form diversity, more desired morphology, or other property described herein.

### BRIEF DESCRIPTION OF THE DRAWINGS

FIGS. 1A-B PXRD diffractograms of a co-crystal comprising celecoxib and nicotinamide, with the background removed and as collected, respectively.

FIG. 2 DSC thermogram for a co-crystal comprising celecoxib and nicotinamide.

FIG. 3 TGA thermogram for a co-crystal comprising celecoxib and nicotinamide.

FIG. 4 Raman spectrum for a co-crystal comprising celecoxib and nicotinamide.

FIGS. 5A-B PXRD diffractograms of a co-crystal comprising celecoxib and 18-crown-6, with the background removed and as collected, respectively.

FIG. 6 DSC thermogram for a co-crystal comprising celecoxib and 18-crown-6.

FIG. 7 TGA thermogram for a co-crystal comprising celecoxib and 18-crown-6.

FIGS. 8A-B PXRD diffractograms of a co-crystal comprising topiramate and 18-crown-6, with the background removed and as collected, respectively.

FIG. 9 DSC thermogram for a co-crystal comprising topiramate and 18-crown-6.

FIGS. 10A-B PXRD diffractograms of a co-crystal comprising olanzapine and nicotinamide (Form I), with the background removed and as collected, respectively.

FIG. 11 DSC thermogram for a co-crystal comprising olanzapine and nicotinamide (Form I).

FIG. 12 PXRD diffractogram of a co-crystal comprising olanzapine and nicotinamide (Form II).

FIGS. 13A-B PXRD diffractograms of a co-crystal comprising olanzapine and nicotinamide (Form III), with the background removed and as collected, respectively.

FIGS. 14A-D Packing diagrams and crystal structure of a co-crystal comprising olanzapine and nicotinamide (Form III).

FIG. 15 PXRD diffractogram of a co-crystal comprising cis-itraconazole and succinic acid.

FIG. 16 DSC thermogram for a co-crystal comprising cis-itraconazole and succinic acid.

FIG. 17 PXRD diffractogram of a co-crystal comprising cis-itraconazole and fumaric acid.

FIG. 18 DSC thermogram for a co-crystal comprising cis-itraconazole and fumaric acid.

FIG. 19 PXRD diffractogram of a co-crystal comprising cis-itraconazole and L-tartaric acid.

FIG. 20 DSC thermogram for a co-crystal comprising cis-itraconazole and L-tartaric acid.

FIGS. 21A-B An acetaminophen 1-D polymeric chain and a co-crystal of acetaminophen and 4,4′-bipyridine, respectively.

FIGS. 22A-B Pure phenyloin and a co-crystal with phenyloin and pyridone, respectively.

FIGS. 23A-D Pure aspirin and the corresponding crystal structure are shown in FIGS. 23A and 23B, respectively. FIGS. 23C and 23D show the supramolecular entity containing the synthon and corresponding co-crystal of aspirin and 4,4′-bipyridine, respectively.

FIGS. 24A-D Pure ibuprofen and the corresponding crystal structure are shown in FIGS. 24A and 24B, respectively. FIGS. 24C and 24D show the supramolecular entity containing the synthon and corresponding co-crystal of ibuprofen and 4,4′-bipyridine, respectively.

FIGS. 25A-D Pure flurbiprofen and the corresponding crystal structure are shown in FIGS. 25A and 25B, respectively. FIGS. 25C and 25D show the supramolecular synthon and corresponding co-crystal of flurbiprofen and 4,4′-bipyridine, respectively.

FIGS. 26A-B The supramolecular entity containing the synthon and the corresponding co-crystal structure of flurbiprofen and trans-1,2-bis(4-pyridyl)ethylene, respectively.

FIGS. 27A-B The crystal structure of pure carbamazepine and the co-crystal structure of carbamazepine and p-phthalaldehyde, respectively.

FIG. 28 A packing diagram of the co-crystal structure of carbamazepine and nicotinamide.

FIG. 29 PXRD diffractogram of a co-crystal comprising carbamazepine and nicotinamide.

FIG. 30 DSC thermogram for a co-crystal comprising carbamazepine and nicotinamide.

FIG. 31 A packing diagram of the co-crystal structure of carbamazepine and saccharin.

FIG. 32 PXRD diffractogram of a co-crystal comprising carbamazepine and saccharin.

FIG. 33 DSC thermogram for a co-crystal comprising carbamazepine and saccharin.

FIGS. 34A-B The crystal structure of carbamazepine and the co-crystal structure of carbamazepine and 2,6-pyridinedicarboxylic acid, respectively.

FIGS. 35A-B The crystal structure of carbamazepine and the co-crystal structure of carbamazepine and 5-nitroisophthalic acid, respectively.

FIGS. 36A-B The crystal structure of carbamazepine and the co-crystal structure of carbamazepine and 1,3,5,7-adamantanetetracarboxylic acid, respectively.

FIGS. 37A-B The crystal structure of carbamazepine and the co-crystal structure of carbamazepine and benzoquinone, respectively.

FIGS. 38A-B The crystal structure of carbamazepine and the co-crystal structure of carbamazepine and trimesic acid, respectively.

FIG. 39 PXRD diffractogram of a co-crystal comprising carbamazepine and trimesic acid.

FIG. 40 Dissolution profile for a co-crystal of celecoxib:nicotinamide vs. celecoxib free acid.

FIG. 41 Dissolution profile for co-crystals of itraconazole:succinic acid, itraconazle:tartaric acid and itraconazole:malic acid vs. itraconazole free base.

FIG. 42 Hygroscopicity profile for a co-crystal of celecoxib:nicotinamide vs. celecoxib sodium.

FIG. 43 Hydrogen-bonding motifs observed in co-crystals.

FIG. 44 Dissolution profile of several formulations of modafinil free form and modafinil:malonic acid (Form I).

### DETAILED DESCRIPTION OF THE INVENTION

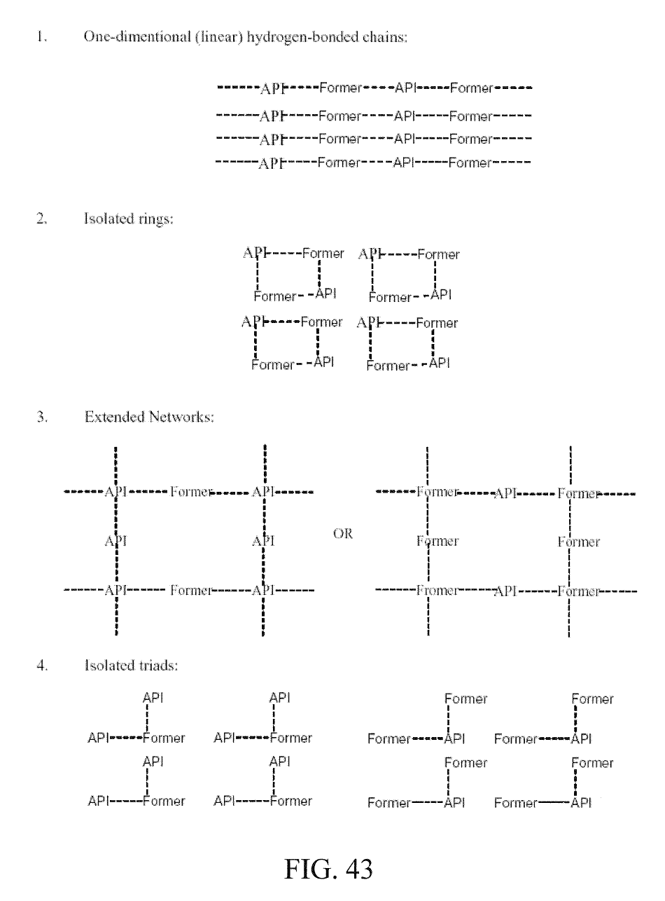

The term “co-crystal” as used herein means a crystalline material comprised of two or more unique solids at room temperature, each containing distinctive physical characteristics, such as structure, melting point and heats of fusion, with the exception that, if specifically stated, the API may be a liquid at room temperature. The co-crystals of the present invention comprise a co-crystal former H-bonded to an API. The co-crystal former may be H-bonded directly to the API or may be H-bonded to an additional molecule which is bound to the API. The additional molecule may be H-bonded to the API or bound ionically or covalently to the API. The additional molecule could also be a different API. Solvates of API compounds that do not further comprise a co-crystal former are not co-crystals according to the present invention. The co-crystals may however, include one or more solvate molecules in the crystalline lattice. That is, solvates of co-crystals, or a co-crystal further comprising a solvent or compound that is a liquid at room temperature, is included in the present invention, but crystalline material comprised of only one solid and one or more liquids (at room temperature) are not included in the present invention, with the previously noted exception of specifically stated liquid APIs. The co-crystals may also be a co-crystal between a co-crystal former and a salt of an API, but the API and the co-crystal former of the present invention are constructed or bonded together through hydrogen bonds. Other modes of molecular recognition may also be present including, pi-stacking, guest-host complexation and van der Waals interactions. Of the interactions listed above, hydrogen-bonding is the dominant interaction in the formation of the co-crystal, (and a required interaction according to the present invention) whereby a non-covalent bond is formed between a hydrogen bond donor of one of the moieties and a hydrogen bond acceptor of the other. Hydrogen bonding can result in several different intermolecular configurations. For example, hydrogen bonds can result in the formation of dimers, linear chains, or cyclic structures. These configurations can further include extended (two-dimensional) hydrogen bond networks and isolated triads (FIG. 43). An alternative embodiment provides for a co-crystal wherein the co-crystal former is a second API. In another embodiment, the co-crystal former is not an API. In another embodiment the co-crystal comprises two co-crystal formers. For purposes of the present invention, the chemical and physical properties of an API in the form of a co-crystal may be compared to a reference compound that is the same API in a different form. The reference compound may be specified as a free form, or more specifically, a free acid, free base, or zwitterion; a salt, or more specifically for example, an inorganic base addition salt such as sodium, potassium, lithium, calcium, magnesium, ammonium, aluminum salts or organic base addition salts, or an inorganic acid addition salts such as HBr, HCl, sulfuric, nitric, or phosphoric acid addition salts or an organic acid addition salt such as acetic, propionic, pyruvic, malanic, succinic, malic, maleic, fumaric, tartaric, citric, benzoic, methanesulfonic, ethanesulforic, stearic or lactic acid addition salt; an anhydrate or hydrate of a free form or salt, or more specifically, for example, a hemihydrate, monohydrate, dihydrate, trihydrate, quadrahydrate, pentahydrate, sesquihydrate; or a solvate of a free form or salt. For example, the reference compound for an API in salt form co-crystallized with a co-crystal former can be the API salt form. Similarly, the reference compound for a free acid API co-crystallized with a co-crystal former can be the free acid API. The reference compound may also be specified as crystalline or amorphous.

According to the present invention, the co-crystals can include an acid addition salt or base addition salt of an API. Acid addition salts include, but are not limited to, inorganic acids such as hydrochloric acid, hydrobromic acid, sulfuric acid, nitric acid, and phosphoric acid, and organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartatic acid, citric acid, benzoic acid, o-(4-hydroxybenzoyl)benzoic acid, cinnamic acid, madelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutaric acid, hydroxynaphthoic acid, salicylic acid, stearic acid, and muconic acid. Base addition salts include, but are not limited to, inorganic bases such as sodium, potassium, lithium, ammonium, calcium and magnesium salts, and organic bases such as primary, secondary and tertiary amines (e.g. isopropylamine, trimethyl amine, diethyl amine, tri(iso-propyl) amine, tri(n-propyl) amine, ethanolamine, 2-dimethylaminoethanol, tromethamine, lysine, arginine, histidine, procaine, hydrabamine, choline, betaine, ethylenediamine, glucosamine, N-alkylglucamines, theobromine, purines, piperazine, piperidine, morpholine, and N-ethylpiperidine).

The ratio of API to co-crystal former may be stoichiometric or non-stoichiometric according to the present invention. For example, 1:1, 1.5:1, 1:1.5, 2:1 and 1:2 ratios of API:co-crystal former are acceptable.

It has surprisingly been found that when an API and a selected co-crystal former are allowed to form co-crystals, the resulting co-crystals give rise to improved properties of the API, as compared to the API in a free form (including free acids, free bases, and zwitterions, hydrates, solvates, etc.), or an acid or base salt thereof particularly with respect to: solubility, dissolution, bioavailability, stability, Cmax, Tmax, processability, longer lasting therapeutic plasma concentration, hygroscopicity, crystallization of amorphous compounds, decrease in form diversity (including polymorphism and crystal habit), change in morphology or crystal habit, etc. For example, a co-crystal form of an API is particularly advantageous where the original API is insoluble or sparingly soluble in water. Additionally, the co-crystal properties conferred upon the API are also useful because the bioavailability of the API can be improved and the plasma concentration and/or serum concentration of the API can be improved. This is particularly advantageous for orally-administrable formulations. Moreover, the dose response of the API can be improved, for example by increasing the maximum attainable response and/or increasing the potency of the API by increasing the biological activity per dosing equivalent.

Accordingly, in a first aspect, the present invention provides a pharmaceutical composition comprising a co-crystal of an API and a co-crystal former, such that the API and co-crystal former are capable of co-crystallizing from a solution phase under crystallization conditions or from the solid-state, for example, through grinding, heating, or through vapor transfer (e.g., co-sublimation). In another aspect, the API has at least one functional group selected from ether, thioether, alcohol, thiol, aldehyde, ketone, thioketone, nitrate ester, phosphate ester, thiophosphate ester, ester, thioester, sulfate ester, carboxylic acid, phosphonic acid, phosphinic acid, sulfonic acid, amide, primary amine, secondary amine, ammonia, tertiary amine, imine, thiocyanate, cyanamide, oxime, nitrile, diazo, organohalide, nitro, S-heterocyclic ring, thiophene, N-heterocyclic ring, pyrrole, O-heterocyclic ring, furan, epoxide, peroxide, hydroxamic acid, imidazole, and pyridine and a co-crystal former which has at least one functional group selected from ether, thioether, alcohol, thiol, aldehyde, ketone, thioketone, nitrate ester, phosphate ester, thiophosphate ester, ester, thioester, sulfate ester, carboxylic acid, phosphonic acid, phosphinic acid, sulfonic acid, amide, primary amine, secondary amine, ammonia, tertiary amine, imine, thiocyanate, cyanamide, oxime, nitrile, diazo, organohalide, nitro, S-heterocyclic ring, thiophene, N-heterocyclic ring, pyrrole, O-heterocyclic ring, furan, epoxide, peroxide, hydroxamic acid, imidazole, and pyridine, or a functional group in a Table herein, such that the API and co-crystal former are capable of co-crystallizing from a solution phase under crystallization conditions.

The co-crystals of the present invention are formed where the API and co-crystal former are bonded together through hydrogen bonds. Other non-covalent interactions, including pi-stacking and van der Waals interactions, may also be present.

In one embodiment, the co-crystal former is selected from the co-crystal formers of Table I and Table II. In other embodiments, the co-crystal former of Table I is specified as a Class 1, Class 2, or Class 3 co-crystal former (see column labeled “class” Table I). In another embodiment, the difference in pKₐ value of the co-crystal former and the API is less than 2. In other embodiments, the difference in pKₐ values of the co-crystal former and API is less than 3, less than 4, less than 5, between 2 and 3, between 3 and 4, or between 4 and 5. Table I lists multiple pKₐ values for co-crystal formers having multiple functionalities. It is readily apparent to one skilled in the art the particular functional group corresponding to a particular pKₐ value.

In another embodiment the particular functional group of a co-crystal former interacting with the API is specified (see for example Table I, columns labeled “Functionality” and “Molecular Structure” and the column of Table II labeled “Co-Crystal Former Functional Group”). In a further embodiment the functional group of the API interacting with the co-crystal former functional group is specified (see, for example, Tables II and III).

In another embodiment, the co-crystal comprises more than one co-crystal former. For example, two, three, four, five, or more co-crystal formers can be incorporated in a co-crystal with an API. Co-crystals which comprise two or more co-crystal formers and an API are bound together via hydrogen bonds. In one embodiment, incorporated co-crystal formers are hydrogen bonded to the API molecules. In another embodiment, co-crystal formers are hydrogen bonded to either the API molecules or the incorporated co-crystal formers.

In a further embodiment, several co-crystal formers can be contained in a single compartment, or kit, for ease in screening an API for potential co-crystal species. The co-crystal kit can comprise 5, 10, 15, 20, 25, 30, 40, 50, 60, 70, 80, 90, 100, or more of the co-crystal formers in Tables I and II. The co-crystal formers are in solid form or in solution and in an array of individual reaction vials such that individual co-crystal formers can be tested with one or more APIs by one or more crystallization methods or multiple co-crystal formers can be easily tested against one or more compounds by one or more crystallization methods. The crystallization methods include, but are not limited to, melt recrystallization, grinding, milling, standing, co-crystal formation from solution by evaporation, thermally driven crystallization from solution, co-crystal formation from solution by addition of anti-solvent, co-crystal formation from solution by vapor-diffusion, co-crystal formation from solution by drown-out, co-crystal formation from solution by any combination of the above mentioned techniques, co-crystal formation by co-sublimation, co-crystal formation by sublimation using a Knudsen cell apparatus, co-crystal formation by standing the desired components of the co-crystal in the presence of solvent vapor, co-crystal formation by slurry conversion of the desired components of the co-crystal in a solvent or mixtures of solvents, or co-crystal formation by any combination of the above techniques in the presence of additives, nucleates, crystallization enhancers, precipitants, chemical stabilizers, or anti-oxidants. The co-crystallization kits can be used alone or as part of larger crystallization experiments. For example, kits can be constructed as single co-crystal former single well kits, single co-crystal former multi-well kits, multi-co-crystal former single well kits, or multi-co-crystal former multi-well kits. High-throughput crystallization (e.g., the CrystalMax™ platform) can be used to construct and customize co-crystal former kits. Multi-well plates (e.g., 96 wells, 384 wells, 1536 wells, etc.), for example, can be used to store or employ an array of co-crystal formers.

In a further embodiment, the API is selected from an API of Table IV or elsewhere herein. For pharmaceuticals listed in Table IV, co-crystals can comprise such APIs in free form (i.e. free acid, free base, zwitter ion), salts, solvates, hydrates, or the like. For APIs in Table IV listed as salts, solvates, hydrates, and the like, the API can either be of the form listed in Table IV or its corresponding free form, or of another form that is not listed. Table IV includes the CAS number, chemical name, or a PCT or patent reference (each incorporated herein in their entireties). In further embodiments, the functional group of the particular API interacting with the co-crystal former is specified. A specific functional group of a co-crystal former, a specific co-crystal former, or a specified functional group or a specific co-crystal former interacting with the particular API may also be specified. It is noted that for Table II, the co-crystal former, and optionally the specific functionality, and each of the listed corresponding interacting groups are included as individual species of the present invention. Thus, each specific combination of a co-crystal former and one of the interacting groups in the same row may be specified as a species of the present invention. The same is true for other combinations as discussed in the Tables and elsewhere herein.

In another embodiment of the present invention, the co-crystal comprises an API wherein the API forms a dimeric primary amide structure via hydrogen bonds with an R²₂ (8) motif. In such a structure, the NH₂ moiety can also participate in a hydrogen bond with a donor or an acceptor moiety from, for example, a co-crystal former or an additional (third) molecule, and the C═O moiety can participate in a hydrogen bond with a donor moiety from the co-crystal former or the additional molecule. In a further embodiment, the dimeric primary amide structure further comprises one, two, three, or four hydrogen bond donors. In a further embodiment, the dimeric primary amide structure further comprises one or two hydrogen bond acceptors. In a further embodiment, the dimeric primary amide structure further comprises a combination of hydrogen bond donors and acceptors. For example, the dimeric primary amide structure can further comprise one hydrogen bond donor and one hydrogen bond acceptor, one hydrogen bond donor and two hydrogen bond acceptors, two hydrogen bond donors and one hydrogen bond acceptor, two hydrogen bond donors and two hydrogen bond acceptors, or three hydrogen bond donors and one hydrogen bond acceptor. Two non-limiting examples of APIs which form a dimeric primary amide co-crystal structure include modafinil and carbamazepine. Some examples of APIs which include a primary amide functional group include, but are not limited to, arotinolol, atenolol, carpipramine, cefotetan, cefsulodin, docapromine, darifenacin, exalamide, fidarestat, frovatriptan, silodosin, levetiracetam, MEN-10700, mizoribine, oxiracetam, piracetam, protirelin, TRH, ribavirin, valrecemide, temozolomide, tiazofurin, antiPARP-2, levovirin, N-benzyloxycarbonyl glycinamide, and UCB-34714.

In each process according to the invention, there is a need to contact the API with the co-crystal former. This may involve grinding or milling the two solids together or melting one or both components and allowing them to recrystallize. The use of a granulating liquid may improve or may impede co-crystal formation. Non-limiting examples of tools useful for the formation of co-crystals may include, for example, an extruder or a mortar and pestle. Further, contacting the API with the co-crystal former may also involve either solubilizing the API and adding the co-crystal former, or solubilizing the co-crystal former and adding the API. Crystallization conditions are applied to the API and co-crystal former. This may entail altering a property of the solution, such as pH or temperature and may require concentration of the solute, usually by removal of the solvent, typically by drying the solution. Solvent removal results in the concentration of both API and co-crystal former increasing over time so as to facilitate crystallization. For example, evaporation, cooling, co-sublimation, or the addition of an antisolvent may be used to crystallize co-crystals. In another embodiment, a slurry comprising an API and a co-crystal former is used to form co-crystals. Once the solid phase comprising any crystals is formed, this may be tested as described herein.

The manufacture of co-crystals on a large and/or commercial scale may be successfully completed using one or more of the processes and techniques described herein. For example, crystallization of co-crystals from a solvent and grinding or milling are conceivable non-limiting processes.

In another embodiment, the use of an excess (more than 1 molar equivalent for a 1:1 co-crystal) of a co-crystal former has been shown to drive the formation of stoichiometric co-crystals. For example, co-crystals with stoichiometries of 1:1, 2:1, or 1:2 can be produced by adding co-crystal former in an amount that is 2, 3, 4, 5, 6, 7, 8, 9, 10, 15, 20, 25, 50, 75, 100 times or more than the stoichiometric amount for a given co-crystal. Such an excessive use of a co-crystal former to form a co-crystal can be employed in solution or when grinding an API and a co-crystal former to drive co-crystal formation.

In another embodiment, the present invention provides for the use of an ionic liquid as a medium for the formation of a co-crystal, and can also be used to crystallize other forms in addition to co-crystals (e.g., salts, solvates, free acid, free base, zwitterions, etc.). This medium is useful, for example, where the above methods do not work or are difficult or impossible to control. Several non-limiting examples of ionic liquids useful in co-crystal formation are: 1-butyl-3-methylimidazolium lactate, 1-ethyl-3-methylimidazolium lactate, and 1-butylpyridinium hexafluorophosphate.

The co-crystals obtained as a result of one or more of the above processes or techniques may be readily incorporated into a pharmaceutical composition by conventional means. Pharmaceutical compositions in general are discussed in further detail below and may further comprise a pharmaceutically-acceptable diluent, excipient or carrier.

In a further aspect, the present invention provides a process for the production of a pharmaceutical composition, which process comprises:

(1) providing an API which has at least one functional group selected from ether, thioether, alcohol, thiol, aldehyde, ketone, thioketone, nitrate ester, phosphate ester, thiophosphate ester, ester, thioester, sulfate ester, carboxylic acid, phosphonic acid, phosphinic acid, sulfonic acid, amide, primary amine, secondary amine, ammonia, tertiary amine, imine, thiocyanate, cyanamide, oxime, nitrile diazo, organohalide, nitro, S-heterocyclic ring, thiophene, N-heterocyclic ring, pyrrole, O-heterocyclic ring, furan, epoxide, peroxide, hydroxamic acid, imidazole, and pyridine or of Table II or III;

(2) providing a co-crystal former which has at least one functional group selected from ether, thioether, alcohol, thiol, aldehyde, ketone, thioketone, nitrate ester, phosphate ester, thiophosphate ester, ester, thioester, sulfate ester, carboxylic acid, phosphonic acid, phosphinic acid, sulfonic acid, amide, primary amine, secondary amine, ammonia, tertiary amine, imine, thiocyanate, cyanamide, oxime, nitrile, diazo, organohalide, nitro, S-heterocyclic ring, thiophene, N-heterocyclic ring, pyrrole, O-heterocyclic ring, furan, epoxide, peroxide, hydroxamic acid, imidazole, and pyridine or of Table I, II, or III;

(3) grinding, heating or contacting in solution the API with the co-crystal former under crystallization conditions;

(4) isolating co-crystals formed thereby; and

(5) incorporating the co-crystals into a pharmaceutical composition.

In a still further aspect the present invention provides a process for the production of a pharmaceutical composition, which comprises:

(1) grinding, heating or contacting in solution an API with a co-crystal former, under crystallization conditions, so as to form a solid phase;

(2) isolating co-crystals comprising the API and the co-crystal former; and

(3) incorporating the co-crystals into a pharmaceutical composition.

Assaying the solid phase for the presence of co-crystals of the API and the co-crystal former may be carried out by conventional methods known in the art. For example, it is convenient and routine to use powder X-ray diffraction techniques to assess the presence of co-crystals. This may be affected by comparing the spectra of the API, the crystal former and putative co-crystals in order to establish whether or not true co-crystals had been formed. Other techniques, used in an analogous fashion, include differential scanning calorimetry (DSC), thermogravimetric analysis (TGA), solid state NMR spectroscopy, and Raman spectroscopy. Single crystal X-ray diffraction is especially useful in identifying co-crystal structures.

In a further aspect, the present invention therefore provides a process of screening for co-crystal compounds, which comprises:

(1) providing (i) an API compound, and (ii) a co-crystal former; and

(2) screening for co-crystals of APIs with co-crystal formers by subjecting each combination of API and co-crystal former to a step comprising:

(a) grinding, heating, co-subliming, co-melting, or contacting in solution the API with the co-crystal former under crystallization conditions so as to form a solid phase; and

(b) isolating co-crystals comprising the API and the co-crystal former.

An alternative embodiment is drawn to a process of screening for co-crystal compounds, which comprises:

(1) providing (i) an API or a plurality of different APIs, and (ii) a co-crystal former or a plurality of different co-crystal formers, wherein at least one of the API and the co-crystal former is provided as a plurality thereof; and

(2) screening for co-crystals of APIs with co-crystal formers by subjecting each combination of API and co-crystal former to a step comprising

(a) grinding, heating, co-subliming, co-melting, or contacting in solution the API with the co-crystal former under crystallization conditions so as to form a solid phase; and

(b) isolating co-crystals comprising the API and the co-crystal former.

Some of the APIs and co-crystal formers of the present invention have one or more chiral centers and may exist in a variety of stereoisomeric configurations. As a consequence of these chiral centers, several APIs and co-crystal formers of the present invention occur as racemates, mixtures of enantiomers and as individual enantiomers, as well as diastereomers and mixtures of diastereomers. All such racemates, enantiomers, and diastereomers are within the scope of the present invention including, for example, cis- and trans-isomers, R- and S-enantiomers, and (D)- and (L)-isomers. Co-crystals of the present invention can include isomeric forms of either the API or the co-crystal former or both. Isomeric forms of APIs and co-crystal formers include, but are not limited to, stereoisomers such as enantiomers and diastereomers. In one embodiment, a co-crystal can comprise a racemic API and/or co-crystal former. In another embodiment, a co-crystal can comprise an enantiomerically pure API and/or co-crystal former. In another embodiment, a co-crystal can comprise an API or a co-crystal former with an enantiomeric excess of about 50 percent, 55 percent, 60 percent, 65 percent, 70 percent, 75 percent, 80 percent, 85 percent, 90 percent, 95 percent, 96 percent, 97 percent, 98 percent, 99 percent, greater than 99 percent, or any intermediate value. Several non-limiting examples of stereoisomeric APIs include modafinil, cis-itraconazole, ibuprofen, and flurbiprofen. Several non-limiting examples of stereoisomeric co-crystal formers include tartaric acid and malic acid.

Co-crystals comprising enantiomerically pure components (e.g., API or co-crystal former) can give rise to chemical and/or physical properties which are modulated with respect to those of the corresponding co-crystal comprising a racemic component. For example, the modafinil:malonic acid co-crystal from Example 10 comprises racemic modafinil.

Enantiomerically pure R-modafinil:malonic acid can conceivably be synthesized via the same or another method of the present invention and is therefore included in the scope of the invention. Likewise, enantiomerically pure S-modafinil:malonic acid can conceivably be synthesized via a method of the present invention and is therefore included in the scope of the invention. A co-crystal comprising an enantiomerically pure component can give rise to a modulation of, for example, activity, bioavailability, or solubility, with respect to the corresponding co-crystal comprising a racemic component. As an example, the co-crystal R-modafinil:malonic acid can have modulated properties as compared to the racemic modafinil:malonic acid co-crystal.

As used herein and unless otherwise noted, the term “racemic co-crystal” refers to a co-crystal which is comprised of an equimolar mixture of two enantiomers of the API, the co-crystal former, or both. For example, a co-crystal comprising a stereoisomeric API and a non-stereoisomeric co-crystal former is a “racemic co-crystal” when there is present an equimolar mixture of the API enantiomers. Similarly, a co-crystal comprising a non-stereoisomeric API and a stereoisomeric co-crystal former is a “racemic co-crystal” when there is present an equimolar mixture of the co-crystal former enantiomers. In addition, a co-crystal comprising a stereoisomeric API and a stereoisomeric co-crystal former is a “racemic co-crystal” when there is present an equimolar mixture of the API enantiomers and of the co-crystal former enantiomers.

As used herein and unless otherwise noted, the term “enantiomerically pure co-crystal” refers to a co-crystal which is comprised of a stereoisomeric API or a stereoisomeric co-crystal former or both where the enantiomeric excess of the stereoisomeric species is greater than or equal to about 90 percent ee.

In another embodiment, the present invention includes a pharmaceutical composition comprising a co-crystal with an enantiomerically pure API or co-crystal former wherein the bioavailability is modulated with respect to the racemic co-crystal. In another embodiment, the present invention includes a pharmaceutical composition comprising a co-crystal with an enantiomerically pure API or co-crystal former wherein the activity is modulated with respect to the racemic co-crystal. In another embodiment, the present invention includes a pharmaceutical composition comprising a co-crystal with an enantiomerically pure API or co-crystal former wherein the solubility is modulated with respect to the racemic co-crystal.

As used herein, the term “enantiomerically pure” includes a composition which is substantially enantiomerically pure and includes, for example, a composition with greater than or equal to about 90, 91, 92, 93, 94, 95, 96, 97, 98, or 99 percent enantiomeric excess.

### Solubility Modulation

In a further aspect, the present invention provides a process for modulating the solubility of an API, which process comprises:

(1) grinding, heating, co-subliming, co-melting, or contacting in solution the API with a co-crystal former under crystallization conditions, so as to form a co-crystal of the API and the co-crystal former; and

(2) isolating co-crystals comprising the API and the co-crystal former.

In one embodiment, the solubility of the API is modulated such that the aqueous solubility is increased. Solubility of APIs may be measured by any conventional means such as chromatography (e.g., HPLC) or spectroscopic determination of the amount of API in a saturated solution of the API, such as UV-spectroscopy, IR-spectroscopy, Raman spectroscopy, quantitative mass spectroscopy, or gas chromatography.

In another aspect of the invention, the API may have low aqueous solubility. Typically, low aqueous solubility in the present application refers to a compound having a solubility in water which is less than or equal to 10 mg/mL, when measured at 37 degrees C., and preferably less than or equal to 5 mg/mL or 1 mg/mL. Low aqueous solubility can further be specifically defined as less than or equal to 900, 800, 700, 600, 500, 400, 300, 200 150 100, 90, 80, 70, 60, 50, 40, 30, 20 micrograms/mL, or further 10, 5 or 1 micrograms/mL, or further 900, 800, 700, 600, 500, 400, 300, 200 150, 100 90, 80, 70, 60, 50, 40, 30, 20, or 10 ng/mL, or less than 10 ng/mL when measured at 37 degrees C. Aqueous solubility can also be specified as less than 500, 400, 300, 200, 150, 100, 75, 50 or 25 mg/mL. As embodiments of the present invention, solubility can be increased 2, 3, 4, 5, 7, 10, 15, 20, 25, 50, 75, 100, 200, 300, 500, 750, 1000, 5000, or 10,000 times by making a co-crystal of the reference form (e.g., crystalline or amorphous free acid, free base or zwitter ion, hydrate or solvate), or a salt thereof. Further aqueous solubility can be measured in simulated gastric fluid (SGF) or simulated intestinal fluid (SIF) rather than water. SGF (non-diluted) of the present invention is made by combining 1 g/L Triton X-100 and 2 g/L NaCl in water and adjusting the pH with 20 mM HCl to obtain a solution with a final pH=1.7 (SIF is 0.68% monobasic potassium phosphate, 1% pancreatin, and sodium hydroxide where the pH of the final solution is 7.5). The pH of the solvent used may also be specified as 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 11.5, 12, 12.5, 13, 13.5, or 14 or any pH in between successive values.

Examples of embodiments includes: co-crystal compositions with an aqueous solubility, at 37 degrees C. and a pH of 7.0, that is increased at least 5 fold over the reference form, co-crystal compositions with a solubility in SGF that is increased at least 5 fold over the reference form, co-crystal compositions with a solubility in SIF that is increased at least 5 fold over the reference form.

### Dissolution Modulation

In another aspect of the present invention, the dissolution profile of the API is modulated whereby the aqueous dissolution rate or the dissolution rate in simulated gastric fluid or in simulated intestinal fluid, or in a solvent or plurality of solvents is increased. Dissolution rate is the rate at which API solids dissolve in a dissolution medium. For APIs whose absorption rates are faster than the dissolution rates (e.g., steroids), the rate-limiting step in the absorption process is often the dissolution rate. Because of a limited residence time at the absorption site, APIs that are not dissolved before they are removed from intestinal absorption site are considered useless. Therefore, the rate of dissolution has a major impact on the performance of APIs that are poorly soluble. Because of this factor, the dissolution rate of APIs in solid dosage forms is an important, routine, quality control parameter used in the API manufacturing process.

Dissolution rate=KS(Cₛ−C)

where K is dissolution rate constant, S is the surface area, Cₛ is the apparent solubility, and C is the concentration of API in the dissolution medium. For rapid API absorption, Cₛ−C is approximately equal to Cₛ. The dissolution rate of APIs may be measured by conventional means known in the art.

The increase in the dissolution rate of a co-crystal, as compared to the reference form (e.g., free form or salt), may be specified, such as by 10, 20, 30, 40, 50, 60, 70, 80, 90, or 100%, or by 2, 3, 4, 5, 6, 7, 8, 9, 10, 15, 20, 25, 30, 40, 50, 75, 100, 125, 150, 175, 200, 250, 300, 350, 400, 500, 1000, 10,000, or 100,000 fold greater than the reference form (e.g., free form or salt form) in the same solution. Conditions under which the dissolution rate is measured is the same as discussed above The increase in dissolution may be further specified by the time the composition remains supersaturated before reaching equilibrium solubility.

Examples of above embodiments include: co-crystal compositions with a dissolution rate in aqueous solution, at 37 degrees C. and a pH of 7.0, that is increased at least 5 fold over the reference form, co-crystal compositions with a dissolution rate in SGF that is increased at least 5 fold over the reference form, co-crystal compositions with a dissolution rate in SIF that is increased at least 5 fold over the reference form.

### Bioavailability Modulation

The methods of the present invention are used to make a pharmaceutical API formulation with greater solubility, dissolution, and bioavailability. Bioavailability can be improved via an increase in AUC, reduced time to Tₘₐₓ, (the time to reach peak blood serum levels), or increased Cₘₐₓ. The present invention can result in higher plasma concentrations of API when compared to the neutral form or salt alone (reference form).

AUC is the area under the plot of plasma concentration of API (not logarithm of the concentration) against time after API administration. The area is conveniently determined by the “trapezoidal rule”: The data points are connected by straight line segments, perpendiculars are erected from the abscissa to each data point, and the sum of the areas of the triangles and trapezoids so constructed is computed. When the last measured concentration (Cₙ, at time tₙ) is not zero, the AUC from tₙ to infinite time is estimated by Cₙ/kₑₗ.

The AUC is of particular use in estimating bioavailability of APIs, and in estimating total clearance of APIs (Cl_{T}). Following single intravenous doses, AUC=D/Cl_{T}, for single compartment systems obeying first-order elimination kinetics, where D is the dose; alternatively, AUC=C₀/kₑₗ, where kₑₗ is the API elimination rate constant. With routes other than the intravenous, for such systems, AUC=F·D/Cl_{T}, where F is the absolute bioavailability of the API.

Thus, in a further aspect, the present invention provides a process for modulating the bioavailability of an API when administered in its normal and effective dose range as a co-crystal, whereby the AUC is increased, the time to Tₘₐₓ is reduced, or Cₘₐₓ is increased, as compared to a reference form, which process comprises:

(1) grinding, heating, co-subliming, co-melting, or contacting in solution the API with a co-crystal former under crystallization conditions, so as to form a co-crystal of the API and the co-crystal former; and

(2) isolating co-crystals comprising the API and the co-crystal former.

Examples of the above embodiments include: co-crystal compositions with a time to Tₘₐₓ that is reduced by at least 10% as compared to the reference form, co-crystal compositions with a time to Tₘₐₓ that is reduced by at least 20% over the reference form, co-crystal compositions with a time to Tₘₐₓ that is reduced by at least 40% over the reference form, co-crystal compositions with a time to Tₘₐₓ that is reduced by at least 50% over the reference form, co-crystal compositions with a Tₘₐₓ that is reduced by at least 60% over the reference form, co-crystal compositions with a Tₘₐₓ that is reduced by at least 70% over the reference form, co-crystal compositions with a Tₘₐₓ that is reduced by at least 80% over the reference form, co-crystal compositions with a Tₘₐₓ that is reduced by at least 90% over the reference form, co-crystal compositions with a Cₘₐₓ that is increased by at least 20% over the reference form, co-crystal compositions with a Cₘₐₓ that is increased by at least 30% over the reference form, co-crystal compositions with a Cₘₐₓ that is increased by at least 40% over the reference form, co-crystal compositions with a Cₘₐₓ that is increased by at least 50% over the reference form, co-crystal compositions with a Cₘₐₓ that is increased by at least 60% over the reference form, co-crystal compositions with a Cₘₐₓ that is increased by at least 70% over the reference form, co-crystal compositions with a Cₘₐₓ that is increased by at least 80% over the reference form, co-crystal compositions with a Cₘₐₓ that is increased by at least 2 fold, 3 fold, 5 fold, 7.5 fold, 10 fold, 25 fold, 50 fold or 100 fold, co-crystal compositions with an AUC that is increased by at least 10% over the reference form, co-crystal compositions with an AUC that is increased by at least 20% over the reference form, co-crystal compositions with an AUC that is increased by at least 30% over the reference form, co-crystal compositions with an AUC that is increased by at least 40% over the reference form, co-crystal compositions with an AUC that is increased by at least 50% over the reference form, co-crystal compositions with an AUC that is increased by at least 60% over the reference form, co-crystal compositions with an AUC that is increased by at least 70% over the reference form, co-crystal compositions with an AUC that is increased by at least 80% over the reference form or co-crystal compositions with an AUC that is increased by at least 2 fold, 3 fold, 4 fold, 5 fold, 6 fold, 7 fold, 8 fold, 9 fold, or 10 fold. Other examples include wherein the reference form is crystalline, wherein the reference form is amorphous, wherein the reference form is an anhydrous crystalline sodium salt, or wherein the reference form is an anhydrous crystalline HCl salt.

### Dose Response Modulation

In a further aspect the present invention provides a process for improving the dose response of an API, which process comprises:

(1) grinding, heating, co-subliming, co-melting, or contacting in solution an API with a co-crystal former under crystallization conditions, so as to form a co-crystal of the API and the co-crystal former; and

(2) isolating co-crystals comprising the API and the co-crystal former.

Dose response is the quantitative relationship between the magnitude of response and the dose inducing the response and may be measured by conventional means known in the art. The curve relating effect (as the dependent variable) to dose (as the independent variable) for an API-cell system is the “dose-response curve”. Typically, the dose-response curve is the measured response to an API plotted against the dose of the API (mg/kg) given. The dose response curve can also be a curve of AUC against the dose of the API given.

In an embodiment of the present invention, a co-crystal of the present invention has an increased dose response curve or a more linear dose response curve than the corresponding reference compound.

### Increased Stability

In a still further aspect the present invention provides a process for improving the stability of an API (as compared to a reference form such as its free form or a salt thereof), which process comprises:

(1) grinding, heating, co-subliming, co-melting, or contacting in solution the pharmaceutical salt with a co-crystal former under crystallization conditions, so as to form a co-crystal of the API and the co-crystal former; and

(2) isolating co-crystals comprising the API and the co-crystal former.

In a preferred embodiment, the compositions of the present invention, including the API or active pharmaceutical ingredient (API) and formulations comprising the API, are suitably stable for pharmaceutical use. Preferably, the API or formulations thereof of the present invention are stable such that when stored at 30 degrees C. for 2 years, less than 0.2% of any one degradant is formed. The term degradant refers herein to product(s) of a single type of chemical reaction. For example, if a hydrolysis event occurs that cleaves a molecule into two products, for the purpose of the present invention, it would be considered a single degradant. More preferably, when stored at 40 degrees C. for 2 years, less than 0.2% of any one degradant is formed. Alternatively, when stored at 30 degrees C. for 3 months, less than 0.2% or 0.15%, or 0.1% of any one degradant is formed, or when stored at 40 degrees C. for 3 months, less than 0.2% or 0.15%, or 0.1% of any one degradant is formed. Further alternatively, when stored at 60 degrees C. for 4 weeks, less than 0.2% or 0.15%, or 0.1% of any one degradant is formed. The relative humidity (RH) may be specified as ambient (RH), 75% (RH), or as any single integer between 1 to 99%.

### Difficult to Salt or Unsaltable Compounds

In a still further aspect the present invention provides a process for making co-crystals of unsaltable or difficult to salt APIs which process comprises:

(1) grinding, heating, co-subliming, co-melting, or contacting in solution an API with a co-crystal former under crystallization conditions, so as to form a co-crystal of the API and the co-crystal former; and

(2) isolating co-crystals comprising the API and the co-crystal former.

Difficult to salt compounds include bases with a pKa less than 3 or acids with a pKa greater than 10. Zwitter ions are also difficult to salt or unsaltable compounds according to the present invention.

### Decreasing Hygroscopicity

In a still further aspect, the present invention provides a method for decreasing the hygroscopicity of an API, which method comprises:

(1) grinding, heating, co-subliming, co-melting, or contacting in solution the API with a co-crystal former under crystallization conditions, so as to form a co-crystal of the API and the co-crystal former; and

(2) isolating co-crystals comprising the API and the co-crystal former.

An aspect of the present invention provides a pharmaceutical composition comprising a co-crystal of an API that is less hygroscopic than amorphous or crystalline, free form or salt (including metal salts such as sodium, potassium, lithium, calcium, magnesium) or another reference compound. Hygroscopicity can be assessed by dynamic vapor sorption analysis, in which 5-50 mg of the compound is suspended from a Cahn microbalance. The compound being analyzed should be placed in a non-hygroscopic pan and its weight should be measured relative to an empty pan composed of identical material and having nearly identical size, shape, and weight. Ideally, platinum pans should be used. The pans should be suspended in a chamber through which a gas, such as air or nitrogen, having a controlled and known percent relative humidity (% RH) is flowed until equilibrium criteria are met. Typical equilibrium criteria include weight changes of less than 0.01% over 3 minutes at constant humidity and temperature. The relative humidity should be measured for samples dried under dry nitrogen to constant weight (<0.01% change in 3 minutes) at 40 degrees C. unless doing so would de-solvate or otherwise convert the material to an amorphous compound. In one aspect, the hygroscopicity of a dried compound can be assessed by increasing the RH from 5 to 95% in increments of 5% RH and then decreasing the RH from 95 to 5% in 5% increments to generate a moisture sorption isotherm. The sample weight should be allowed to equilibrate between each change in % RH. If the compound deliquesces or becomes amorphous above 75% RH, but below 95% RH, the experiment should be repeated with a fresh sample and the relative humidity range for the cycling should be narrowed to 5-75% RH or 10-75% RH, instead of 5-95% RH. If the sample cannot be dried prior to testing due to lack of form stability, than the sample should be studied using two complete humidity cycles of either 10-75% RH or 5-95% RH, and the results of the second cycle should be used if there is significant weight loss at the end of the first cycle.

Hygroscopicity can be defined using various parameters. For purposes of the present invention, a non-hygroscopic molecule should not gain or lose more than 1.0%, or more preferably, 0.5% weight at 25 degrees C. when cycled between 10 and 75% RH (relative humidity at 25 degrees C.). The non-hygroscopic molecule more preferably should not gain or lose more than 1.0%, or more preferably, 0.5% weight when cycled between 5 and 95% RH at 25 degrees C., or more than 0.25% of its weight between 10 and 75% RH. Most preferably, a non-hygroscopic molecule will not gain or lose more than 0.25% of its weight when cycled between 5 and 95% RH.

Alternatively, for purposes of the present invention, hygroscopicity can be defined using the parameters of Callaghan et al., “Equilibrium moisture content of pharmaceutical excipients”, in Api Dev. Ind. Pharm., Vol. 8, pp. 335-369 (1982). Callaghan et al. classified the degree of hygroscopicity into four classes.

Class 1: Non-hygroscopic Essentially no moisture increases occur at relative humidities below 90%.

Class 2: Slightly hygroscopic Essentially no moisture increases occur at relative humidities below 80%.

Class 3: Moderately hygroscopic Moisture content does not increase more than 5% after storage for 1 week at relative humidities below 60%.

Class 4: Very hygroscopic Moisture content increase may occur at relative humidities as low as 40 to 50%.

Alternatively, for purposes of the present invention, hygroscopicity can be defined using the parameters of the European Pharmacopoeia Technical Guide (1999, p. 86) which has defined hygrospocity, based on the static method, after storage at 25 degrees C. for 24 hours at 80% RH:

Slightly hygroscopic: Increase in mass is less than 2 percent m/m and equal to or greater than 0.2 percent m/m.

Hygroscopic: Increase in mass is less than 15 percent m/m and equal to or greater than 0.2 percent m/m.

Very Hygroscopic: Increase in mass is equal to or greater than 15 percent m/m.

Deliquescent: Sufficient water is absorbed to form a liquid.

Co-crystals of the present invention can be set forth as being in Class 1, Class 2, or Class 3, or as being Slightly hygroscopic, Hygroscopic, or Very Hygroscopic. Co-crystals of the present invention can also be set forth based on their ability to reduce hygroscopicity. Thus, preferred co-crystals of the present invention are less hygroscopic than a reference compound. The reference compound can be specified as the API in free form (free acid, free base, hydrate, solvate, etc.) or salt (e.g., especially metal salts such as sodium, potassium, lithium, calcium, or magnesium). Further included in the present invention are co-crystals that do not gain or lose more than 1.0% weight at 25 degrees C. when cycled between 10 and 75% RH, wherein the reference compound gains or loses more than 1.0% weight under the same conditions. Further included in the present invention are co-crystals that do not gain or lose more than 0.5% weight at 25 degrees C. when cycled between 10 and 75% RH, wherein the reference compound gains or loses more than 0.5% or more than 1.0% weight under the same conditions. Further included in the present invention are co-crystals that do not gain or lose more than 1.0% weight at 25 degrees C. when cycled between 5 and 95% RH, wherein the reference compound gains or loses more than 1.0% weight under the same conditions. Further included in the present invention are co-crystals that do not gain or lose more than 0.5% weight at 25 degrees C. when cycled between 5 and 95% RH, wherein the reference compound gains or loses more than 0.5% or more than 1.0% weight under the same conditions. Further included in the present invention are co-crystals that do not gain or lose more than 0.25% weight at 25 degrees C. when cycled between 5 and 95% RH, wherein the reference compound gains or loses more than 0.5% or more than 1.0% weight under the same conditions.

Further included in the present invention are co-crystals that have a hygroscopicity (according to Callaghan et al.) that is at least one class lower than the reference compound or at least two classes lower than the reference compound. Included are a Class 1 co-crystal of a Class 2 reference compound, a Class 2 co-crystal of a Class 3 reference compound, a Class 3 co-crystal of a Class 4 reference compound, a Class 1 co-crystal of a Class 3 reference compound, a Class 1 co-crystal of a Class 4 reference compound, or a Class 2 co-crystal of a Class 4 reference compound.

Further included in the present invention are co-crystals that have a hygroscopicity (according to the European Pharmacopoeia Technical Guide) that is at least one class lower than the reference compound or at least two classes lower than the reference compound. Non-limiting examples include; a slightly hygroscopic co-crystal of a hygroscopic reference compound, a hygroscopic co-crystal of a very hygroscopic reference compound, a very hygroscopic co-crystal of a deliquescent reference compound, a slightly hygroscopic co-crystal of a very hygroscopic reference compound, a slightly hygroscopic co-crystal of a deliquescent reference compound, and a hygroscopic co-crystal of a deliquescent reference compound.

### Crystallizing Amorphous Compounds

In a further aspect, the present invention provides a process for crystallizing an amorphous compound, which process comprises:

(1) grinding, heating, co-subliming, co-melting, or contacting in solution the API with a co-crystal former under crystallization conditions, so as to form a co-crystal of the API and the co-crystal former; and

(2) isolating co-crystals comprising the API and the co-crystal former.

An amorphous compound includes compounds that do not crystallize using routine methods in the art.

### Decreasing Form Diversity

In a still further embodiment aspect the present invention provides a process for reducing the form diversity of an API, which process comprises:

(1) grinding, heating, co-subliming, co-melting, or contacting in solution the API with a co-crystal former under crystallization conditions, so as to form a co-crystal of the API and the co-crystal former; and

(2) isolating co-crystals comprising the API and the co-crystal former.

For purposes of the present invention, the number of forms of a co-crystal is compared to the number of forms of a reference compound (e.g. the free form or a salt of the API) that can be made using routine methods in the art.

### Morphology Modulation

In a still further aspect the present invention provides a process for modifying the morphology of an API, which process comprises:

(1) grinding, heating, co-subliming, co-melting, or contacting in solution the API with a co-crystal former under crystallization conditions, so as to form a co-crystal of the API and the co-crystal former; and

(2) isolating co-crystals comprising the API and the co-crystal former.

In an embodiment the co-crystal comprises or consists of a co-crystal former and a pharmaceutical wherein the interaction between the two, e.g., H-bonding, occurs between a functional group of Table III of an API with a corresponding interacting group of Table III. In a further embodiment, the co-crystal comprises a co-crystal former of Table I or II and an API with a corresponding interacting group of Table III. In a further embodiment the co-crystal comprises an API from Table IV and a co-crystal former with a functional group of Table III. In a further embodiment, the co-crystal is from Table I or II. In an aspect of the invention, only co-crystals having an H-bond acceptor on the first molecule and an H-bond donor on the second molecule, where the first and second molecules are either co-crystal former and API respectively or API and co-crystal former respectively, are included in the present invention. Table IV includes the CAS number, chemical name or a PCT or patent reference (each incorporated herein in their entireties). Thus, whether a particular API contains an H-bond donor, acceptor or both is readily apparent.

In another embodiment, the co-crystal former and API each have only one H-bond donor/acceptor. In another aspect, the molecular weight of the API is less than 2000, 1500, 1000, 750, 500, 350, 200, or 150 Daltons. In another embodiment, the molecular weight of the API is between 100-200, 200-300, 300-400, 400-500, 500-600, 600-700, 700-800, 800-900, 900-1000, 1000-1200, 1200-1400, 1400-1600, 1600-1800, or 1800-2000. APIs with the above molecular weights may also be specifically excluded from the present invention.

The hydrogen bond donor moieties of a co-crystal can include, but are not limited to, any one, any two, any three, any four, or more of the following: amino-pyridine, primary amine, secondary amine, sulfonamide, primary amide, secondary amide, alcohol, and carboxylic acid. The hydrogen bond acceptor moieties of a co-crystal can include, but are not limited to, any one, any two, any three, any four, or more of the following: amino-pyridine, primary amine, secondary amine, sulfonamide, primary amide, secondary amide, alcohol, carboxylic acid, carbonyl, cyano, dimethoxyphenyl, sulfonyl, aromatic nitrogen (6 membered ring), ether, chloride, organochloride, bromide, organobromide, and organoiodide. Hydrogen bonds are known to form many supramolecular structures including, but not limited to, a catemer, a dimer, a trimer, a tetramer, or a higher order structure. Tables V-XXI list specific hydrogen bond donor and acceptor moieties and their approximate interaction distances from the electromagnetic donor atom through the hydrogen atom to the electromagnetic acceptor atom. For example, Table V lists functional groups that are known to hydrogen bond with amino-pyridines. Amino-pyridines comprise two distinct sites of hydrogen bond donation/acceptance. Both the aromatic nitrogen atom (Npy) and the amine group (NH₂) can participate in hydrogen bonds. The ability of a given functional group to participate in a hydrogen bond as a donor or as an acceptor or both can be determined by inspection by those skilled in the art.

The data included in Tables V-XXI are taken from an analysis of solid-state structures as reported in the Cambridge Structural Database (CSD). These data include a number of hydrogen bonding interactions between many functional groups and their associated interaction distances.

TABLE V
Hydrogen bonding functional groups with amino-
pyridines and associated interaction distances

| | Interaction Distances | | Standard |
|---|---|---|---|
| Functional Group | (angstroms) | Mean | Deviation |
| Primary Amide (to NH₂) | 3.07 | N/A | N/A |
| Primary Amide (to Npy) | 2.97 | N/A | N/A |
| Secondary Amide (to NH₂) | 2.75-3.17 | N/A | N/A |
| Secondary Amide (to Npy) | 2.70-3.20 | 2.92 | 0.07 |
| Carboxylic Acid (to NH₂) | 2.72-3.07 | 2.89 | 0.08 |
| Carboxylic Acid (to Npy) | 2.54-2.82 | 2.67 | 0.05 |
| Water (to NH₂) | 2.72-3.15 | 2.94 | 0.09 |
| Water (to Npy) | 2.65-3.15 | 2.87 | 0.10 |
| Alcohol (to NH₂) | 2.78-3.14 | 2.96 | 0.08 |
| Alcohol (to Npy) | 2.63-3.06 | 2.79 | 0.07 |
| Primary Amine | 2.85-3.25 | 3.05 | 0.07 |
| Secondary Amine | 2.83-3.25 | 2.93 | 0.05 |
| Carbonyl | 2.87-3.10 | 2.95 | 0.07 |
| Sulfoxo | 2.70-3.10 | 2.90 | 0.08 |
| Ether | 2.84-3.20 | 3.05 | 0.07 |
| Ester (C—O—C) | 3.09 | N/A | N/A |
| Ester (C═O) | 2.85-3.16 | 3.00 | 0.08 |
| Aromatic N | 2.78-3.25 | 3.04 | 0.07 |
| Cyano | 2.83-3.30 | 3.09 | 0.12 |
| Nitro | 2.85-3.28 | 3.08 | 0.11 |
| Chloride | 3.10-3.45 | 3.25 | 0.08 |
| Bromide | 3.27-3.48 | 3.39 | 0.05 |

TABLE VI
Hydrogen bonding functional groups with primary
amines and associated interaction distances

| | Interaction Distances | | Standard |
|---|---|---|---|
| Functional Group | (angstroms) | Mean | Deviation |
| Primary Amide | 2.73-3.20 | 2.98 | 0.13 |
| Secondary Amide | 2.65-3.20 | 2.97 | 0.09 |
| Carboxylic Acid (O═C) | 2.74-3.15 | 2.94 | 0.09 |
| Carboxylic Acid (OH) | 2.72-3.12 | 2.95 | 0.11 |
| Amino-pyridine | 3.10-3.24 | 3.22 | 0.02 |
| Sulfonamide | 2.86-3.17 | 3.02 | 0.11 |
| Water | 2.65-3.17 | 2.95 | 0.10 |
| Alcohol | 2.63-3.26 | 2.98 | 0.15 |
| Carbonyl | 2.64-3.15 | 2.95 | 0.09 |
| Sulfoxo | 2.70-3.10 | 2.92 | 0.09 |
| Sulfonyl | 2.93-3.12 | 3.13 | 0.12 |
| Ether | 2.75-3.25 | 3.05 | 0.11 |
| Ester (C—O—C) | 2.90-3.20 | 3.11 | 0.07 |
| Ester (O═C) | 2.74-3.27 | 3.04 | 0.12 |
| Aromatic N | 2.92-3.26 | 3.07 | 0.07 |
| Cyano | 2.83-3.30 | 3.02 | 0.06 |
| Nitro | 2.75-3.17 | 3.05 | 0.08 |
| Chloride | 3.07-3.50 | 3.28 | 0.09 |
| Bromide | 3.23-3.60 | 3.43 | 0.08 |

TABLE VII
Hydrogen bonding functional groups with primary
sulfonamides and associated interaction distances

| | Interaction Distances | | Standard |
|---|---|---|---|
| Functional Group | (angstroms) | Mean | Deviation |
| Water | 2.87 | N/A | N/A |
| Alcohol | 2.85-3.07 | 2.94 | 0.06 |
| Primary Amine | 2.85-3.20 | 3.02 | 0.10 |
| Secondary Amine | 2.85-3.20 | 3.03 | 0.10 |
| Sulfonyl | 2.85-3.20 | 3.03 | 0.12 |
| Ether | 2.90-3.20 | 3.07 | 0.08 |
| Ester | 2.85-3.12 | 2.99 | 0.07 |
| Cyano | 3.00 | N/A | N/A |
| Nitro | 3.00-3.20 | 3.12 | 0.07 |
| Chloride | 3.20-3.32 | 3.26 | 0.03 |

TABLE VIII
Hydrogen bonding functional groups with primary
amides and associated interaction distances

| | Interaction Distances | | Standard |
|---|---|---|---|
| Functional Group | (angstroms) | Mean | Deviation |
| Secondary Amide | 2.70-3.15 | 2.935 | 0.07 |
| Carboxylic Acid (OH) | 2.40-2.80 | 2.560 | 0.06 |
| Carboxylic Acid (C═O) | 2.80-3.25 | 2.961 | 0.09 |
| Amino-pyridine (NH₂) | 2.90-3.20 | 3.069 | 0.00 |
| Amino-pyridine (Aromatic N) | 2.80-3.10 | 2.972 | 0.00 |
| Aromatic N | 2.90-3.21 | 3.069 | 0.07 |
| Water (to C═O) | 2.60-3.00 | 2.813 | 0.08 |
| Water (to NH₂) | 2.70-3.07 | 2.945 | 0.07 |
| Alcohol (to C═O) | 2.50-3.00 | 2.753 | 0.07 |
| Alcohol (to NH₂) | 2.70-3.10 | 2.965 | 0.06 |
| Secondary Amine (to C═O) | 2.80-3.10 | 2.967 | 0.07 |
| Secondary Amine (to NH₂) | 3.00-3.15 | 3.079 | 0.03 |
| Carbonyl | 2.80-3.15 | 2.993 | 0.08 |
| Sulfonyl | 2.90-3.00 | 2.920 | 0.00 |
| Ether | 2.80-3.10 | 2.960 | 0.07 |
| Ester (C═O) | 2.70-3.05 | 2.932 | 0.05 |
| Cyano | 3.00-3.30 | 3.117 | 0.07 |
| Nitro | 2.90-3.07 | 3.020 | 0.03 |
| Chloride | 3.10-3.60 | 3.340 | 0.08 |
| Bromide | 3.30-3.80 | 3.550 | 0.11 |

TABLE IX
Hydrogen bonding functional groups with secondary
amides and associated interaction distances

| | Interaction Distances | | Standard |
|---|---|---|---|
| Functional Group | (angstroms) | Mean | Deviation |
| Primary Amide | 2.70-3.15 | 2.935 | 0.07 |
| Carboxylic Acid (C═O) | 2.70-3.10 | 2.920 | 0.09 |
| Carboxylic Acid (OH) | 2.40-3.05 | 2.606 | 0.05 |
| Amino-pyridine (Aromatic N) | 2.70-3.20 | 2.920 | 0.07 |
| Amino-pyridine (NH₂) | 2.75-3.17 | 2.920 | 0.08 |
| Sulfonamide (S═O) | 2.80-3.20 | 3.110 | 0.16 |
| Sulfonamide (NH₂) | 2.70-3.00 | 2.916 | 0.05 |
| Aromatic N | 2.60-3.15 | 2.955 | 0.09 |
| Water (to C═O) | 2.40-3.10 | 2.840 | 0.09 |
| Water (to NH₂) | 2.60-3.10 | 2.887 | 0.10 |
| Alcohol (to C═O) | 2.50-3.04 | 2.773 | 0.09 |
| Alcohol (to NH₂) | 2.50-3.20 | 2.933 | 0.11 |
| Primary Amine | 2.65-3.20 | 2.970 | 0.09 |
| Secondary Amine | 2.60-3.15 | 2.932 | 0.11 |
| Carbonyl | 2.70-3.07 | 2.937 | 0.08 |
| Sulfonyl | 2.60-3.25 | 3.080 | 0.09 |
| Ether | 2.70-3.16 | 2.992 | 0.09 |
| Ester | 2.80-3.16 | 2.986 | 0.09 |
| Cyano | 2.90-3.30 | 3.120 | 0.09 |
| Nitro | 2.80-3.10 | 2.993 | 0.08 |
| Chloride | 2.90-3.40 | 3.261 | 0.15 |
| Bromide | 3.10-3.50 | 3.394 | 0.11 |

TABLE X
Hydrogen bonding functional groups with alcohols
and associated interaction distances

| | Interaction Distances | | Standard |
|---|---|---|---|
| Functional Group | (angstroms) | Mean | Deviation |
| Primary Amide (C═O) | 2.50-3.00 | 2.753 | 0.07 |
| Primary Amide (NH₂) | 2.70-3.10 | 2.965 | 0.06 |
| Secondary Amide (C═O) | 2.50-3.04 | 2.773 | 0.09 |
| Secondary Amide (NH₂) | 2.50-3.20 | 2.933 | 0.11 |
| Carboxylic Acid (C═O) | 2.50-3.00 | 2.792 | 0.08 |
| Carboxylic Acid (OH) | 2.40-2.90 | 2.649 | 0.05 |
| Amino-pyridine (Aromatic N) | 2.60-3.06 | 2.790 | 0.07 |
| Amino-pyridine (NH₂) | 2.75-3.15 | 2.960 | 0.08 |
| Sulfonamide | 2.80-3.07 | 2.940 | 0.06 |
| Aromatic N | 2.50-3.00 | 2.777 | 0.08 |
| Water | 2.40-3.03 | 2.787 | 0.10 |
| Primary Amine | 2.60-3.15 | 2.897 | 0.13 |
| Secondary Amine | 2.60-3.15 | 2.888 | 0.13 |
| Carbonyl | 2.40-3.05 | 2.805 | 0.11 |
| Sulfonyl | 2.40-3.15 | 2.870 | 0.10 |
| Ether | 2.40-3.00 | 2.841 | 0.08 |
| Ester | 2.50-3.10 | 2.852 | 0.10 |
| Cyano | 2.40-3.10 | 2.873 | 0.09 |
| Nitro | 2.45-3.05 | 2.935 | 0.08 |
| Chloride | 2.60-3.30 | 3.093 | 0.07 |
| Bromide | 3.00-3.50 | 3.258 | 0.07 |

TABLE XI
Hydrogen bonding functional groups with carboxylic
acids and associated interaction distances

| | Interaction Distances | | Standard |
|---|---|---|---|
| Functional Group | (angstroms) | Mean | Deviation |
| Primary Amide (NH₂) | 2.80-3.25 | 2.961 | 0.09 |
| Primary Amide (C═O) | 2.40-2.80 | 2.560 | 0.07 |
| Secondary Amide (NH) | 2.70-3.10 | 2.920 | 0.09 |
| Secondary Amide (C═O) | 2.40-3.05 | 2.606 | 0.05 |
| Amino-pyridine (Aromatic N) | 2.50-2.80 | 2.670 | 0.05 |
| Amino-pyridine (NH₂) | 2.70-3.00 | 2.890 | 0.08 |
| Aromatic N | 2.54-2.94 | 2.658 | 0.06 |
| Water (to C═O) | 2.50-3.00 | 2.830 | 0.07 |
| Water (to OH) | 2.40-3.00 | 2.626 | 0.11 |
| Alcohol (to C═O) | 2.50-3.00 | 2.792 | 0.08 |
| Alcohol (to OH) | 2.50-2.90 | 2.649 | 0.05 |
| Primary Amine (to C═O) | 2.70-3.10 | 2.959 | 0.09 |
| Primary Amine (to OH) | 2.70-3.10 | 2.828 | 0.12 |
| Secondary Amine (to C═O) | 2.70-3.10 | 2.909 | 0.11 |
| Secondary Amine (to OH) | 2.70-3.10 | 2.727 | 0.12 |
| Carbonyl | 2.40-3.00 | 2.696 | 0.08 |
| Ether | 2.50-3.00 | 2.751 | 0.12 |
| Ester (C═O) | 2.40-3.05 | 2.672 | 0.07 |
| Ester (C—O—C) | 2.40-3.10 | 2.990 | N/A |
| Cyano | 2.50-2.80 | 2.746 | 0.09 |
| Nitro | 2.70-3.05 | 2.942 | 0.10 |
| Chloride | 2.80-3.20 | 3.001 | 0.05 |
| Bromide | 3.00-3.30 | 3.150 | 0.05 |

TABLE XII
Hydrogen bonding functional groups with carbonyls
and associated interaction distances

| | Interaction Distances | | Standard |
|---|---|---|---|
| Functional Group | (angstroms) | Mean | Deviation |
| Primary Amide | 2.83-3.15 | 3.96 | 0.06 |
| Secondary Amide | 2.70-3.07 | 2.93 | 0.08 |
| Carboxylic Acid | 2.40-3.00 | 2.70 | 0.08 |
| Amino-pyridine | 2.87-3.10 | 2.95 | 0.07 |
| Secondary Sulfonamide | 2.76-3.22 | 2.949 | 0.12 |
| Water | 2.55-3.05 | 2.82 | 0.10 |
| Alcohol | 2.40-3.05 | 2.80 | 0.01 |
| Primary Amine | 2.64-3.15 | 2.959 | 0.09 |
| Secondary Amine | 2.64-3.15 | 2.87 | 0.01 |

TABLE XIII
Hydrogen bonding functional groups with cyano
groups and associated interaction distances

| | Interaction Distances | | Standard |
|---|---|---|---|
| Functional Group | (angstroms) | Mean | Deviation |
| Primary Amide | 3.01-3.30 | 3.15 | 0.09 |
| Secondary Amide | 2.90-3.30 | 3.13 | N/A |
| Carboxylic Acid | 2.57-3.00 | 2.75 | 0.09 |
| Amino-pyridine | 2.84-3.33 | 3.10 | 0.12 |
| Primary Sulfonamide | 2.99 | N/A | N/A |
| Secondary Sulfonamide | 2.83-3.00 | 2.90 | 0.07 |
| Water | 2.78-3.20 | 2.98 | 0.01 |
| Alcohol | 2.72-3.13 | 2.89 | 0.09 |
| Primary Amine | 2.84-3.27 | 3.08 | 0.09 |
| Secondary Amine | 2.84-3.30 | 3.09 | 0.12 |

TABLE XIV
Hydrogen bonding functional groups with sulfonyl
groups and associated interaction distances

| | Interaction Distances | | Standard |
|---|---|---|---|
| Functional Group | (angstroms) | Mean | Deviation |
| Primary Amide | 2.92 | N/A | N/A |
| Secondary Amide | 2.95-3.25 | 3.08 | 0.09 |
| Primary Sulfonamide | 2.85-3.10 | 3.00 | 0.10 |
| Secondary Sulfonamide | 2.85-3.20 | 3.04 | N/A |
| Water | 2.84-3.00 | 2.90 | 0.05 |
| Alcohol | 2.65-3.15 | 2.87 | 0.1 |
| Primary Amine | 2.93-3.32 | 3.13 | 0.12 |
| Secondary Amine | 2.75-3.32 | 3.05 | 0.12 |

TABLE XV
Hydrogen bonding functional groups with aromatic
N and associated interaction distances

| | Interaction Distances | | Standard |
|---|---|---|---|
| Functional Group | (angstroms) | Mean | Deviation |
| Primary Amide | 2.90-3.21 | 3.07 | 0.07 |
| Secondary Amide | 2.60-3.15 | 2.96 | 0.09 |
| Carboxylic Acid | 2.54-2.94 | 2.66 | 0.06 |
| Amino-pyridine | 2.70-3.20 | 3.04 | 0.07 |
| Water | 2.60-3.15 | 2.91 | 0.09 |
| Alcohol | 2.50-3.00 | 2.78 | 0.08 |
| Primary Amine | 2.92-3.26 | 3.07 | 0.07 |
| Secondary Amine | 2.73-3.25 | 3.02 | 0.10 |

TABLE XVI
Hydrogen bonding functional groups with ethers
and associated interaction distances

| | Interaction Distances | | Standard |
|---|---|---|---|
| Functional Group | (angstroms) | Mean | Deviation |
| Primary Amide | 2.80-3.10 | 2.97 | 0.08 |
| Secondary Amide | 2.70-3.16 | 2.99 | 0.09 |
| Carboxylic Acid | 2.50-3.02 | 2.75 | 0.12 |
| Amino-pyridine | 2.80-3.20 | 3.05 | 0.07 |
| Sulfonamide | 0-3.20 | 3.07 | 0.08 |
| Water | 2.40-3.15 | 2.94 | 0.12 |
| Alcohol | 2.40-3.00 | 2.84 | 0.08 |
| Primary Amine | 2.75-3.25 | 3.05 | 0.11 |
| Secondary Amine | 2.60-3.25 | 3.05 | 0.13 |

TABLE XVII
Hydrogen bonding functional groups with chlorides
and associated interaction distances

| | Interaction Distances | | Standard |
|---|---|---|---|
| Functional Group | (angstroms) | Mean | Deviation |
| Primary Amide | 3.10-3.60 | 3.34 | 0.08 |
| Secondary Amide | 2.90-3.30 | 3.18 | 0.06 |
| Carboxylic Acid | 2.80-3.30 | 3.00 | 0.05 |
| Amino-pyridine | 3.10-3.45 | 3.25 | 0.08 |
| Sulfonamide | 0-3.35 | 3.26 | 0.03 |
| Water | 2.70-3.30 | 3.17 | 0.06 |
| Alcohol | 2.50-3.30 | 3.09 | 0.07 |
| Primary Amine | 3.00-3.50 | 3.28 | 0.09 |
| Secondary Amine | 2.90-3.40 | 3.20 | 0.10 |

TABLE XVIII
Hydrogen bonding functional groups with organochlorides
and associated interaction distances

| | Interaction Distances | | Standard |
|---|---|---|---|
| Functional Group | (angstroms) | Mean | Deviation |
| Primary Amide | 3.18-3.21 | 3.20 | 0.02 |
| Secondary Amide | 3.20-3.27 | 3.25 | 0.03 |
| Carboxylic Acid | 2.90-3.23 | 3.17 | 0.07 |
| Amino-pyridine | 3.28-3.33 | 3.31 | 0.03 |
| Sulfonamide | 0-3.50 | N/A | N/A |
| Water | 2.79-3.26 | 3.14 | 0.15 |
| Alcohol | 2.90-3.29 | 3.17 | 0.09 |
| Primary Amine | 3.21-3.29 | 3.25 | 0.05 |
| Secondary Amine | 3.26-3.30 | 3.28 | 0.02 |

TABLE XIX
Hydrogen bonding functional groups with bromides
and associated interaction distances

| | Interaction Distances | | Standard |
|---|---|---|---|
| Functional Group | (angstroms) | Mean | Deviation |
| Primary Amide | 3.30-3.80 | 3.55 | 0.11 |
| Secondary Amide | 3.10-3.80 | 3.39 | 0.11 |
| Carboxylic Acid | 3.00-3.30 | 3.15 | 0.05 |
| Amino-pyridine | 3.20-3.50 | 3.39 | 0.05 |
| Alcohol | 3.00-3.50 | 3.26 | 0.07 |
| Primary Amine | 3.20-3.60 | 3.43 | 0.08 |
| Secondary Amine | 3.10-3.60 | 3.38 | 0.10 |

TABLE XX
Hydrogen bonding functional groups with organobromides
and associated interaction distances

| | Interaction Distances | | Standard |
|---|---|---|---|
| Functional Group | (angstroms) | Mean | Deviation |
| Primary Amide | 0-3.50 | 3.24 | N/A |
| Secondary Amide | 0-3.50 | N/A | N/A |
| Carboxylic Acid | 3.01-3.31 | 3.20 | 0.16 |
| Amino-pyridine | 0-3.50 | 3.38 | N/A |
| Sulfonamide | 0-3.50 | N/A | N/A |
| Water | 3.14-3.27 | 3.21 | 0.09 |
| Alcohol | 2.90-3.36 | 3.21 | 0.12 |
| Primary Amine | 0-3.50 | 3.38 | N/A |
| Secondary Amine | 3.20-3.39 | 3.30 | 0.12 |

TABLE XXI
Hydrogen bonding functional groups with organoiodides
and associated interaction distances

| | Interaction Distances | | Standard |
|---|---|---|---|
| Functional Group | (angstroms) | Mean | Deviation |
| Primary Amide | 0-3.80 | N/A | N/A |
| Secondary Amide | 0-3.80 | N/A | N/A |
| Carboxylic Acid | 0-3.80 | 3.59 | 0.16 |
| Amino-pyridine | 0-3.80 | 3.42 | N/A |
| Aromatic N | 2.70-3.23 | 2.95 | 0.11 |
| Alcohol | 2.90-3.48 | 3.20 | 0.20 |
| Primary Amine | 3.25-3.42 | 3.34 | 0.11 |
| Secondary Amine | 2.71-2.87 | 2.79 | 0.08 |

In another embodiment, peptides, proteins, nucleic acids or other biological APIs are excluded from the present invention. In another embodiment, all non-pharmaceutically acceptable co-crystal formers are excluded from the present invention. In another embodiment, organometallic APIs are excluded from the present invention. In another embodiment, a co-crystal former comprising any one or more of the functional groups of Table III may be specifically excluded from the present invention. In another embodiment, any one or more of the co-crystal formers of Table I or II may be specifically excluded from the present invention. Any APIs currently known in the art may also be specifically excluded from the present invention. For example, carbamazepine, itraconazole, nabumetone, fluoxetine, acetaminophen and theophylline can each be specifically excluded from the present invention. In another embodiment, the API is not a salt, is not a non-metal salt, or is not a metal salt, e.g., sodium, potassium, lithium, calcium or magnesium. In another embodiment, the API is a salt, is a non-metal salt, or is a metal salt, e.g., sodium, potassium, lithium, calcium, magnesium. In one embodiment, the API does not contain a halogen. In one embodiment, the API does contain a halogen.

In another embodiment, any one or more of the APIs of Table IV may be specifically excluded from the present invention. Any APIs currently known in the art may also be specifically excluded from the present invention. For example, nabumetone:2,3-naphthalenediol, fluoxetine HCl:benzoic acid, fluoxetine HCl:succinic acid, acetaminophen:piperazine, acetaminophen:theophylline, theophylline:salicylic acid, theophylline:p-hydroxybenzoic acid, theophylline:sorbic acid, theophylline:1-hydroxy-2-naphthoic acid, theophylline:glycolic acid, theophylline:2,5-dihydroxybenzoic acid, theophylline:chloroacetic acid, bis(diphenylhydantoin):9-ethyladenine acetylacetone solvate, bis(diphenylhydantoin):9-ethyladenine 2,4-pentanedione solvate, 5,5-diphenylbarbituric acid:9-ethyladenine, bis(diphenylhydantoin):9-ethyladenine, 4-aminobenzoic acid:4-aminobenzonitrile, sulfadimidine:salicylic acid, 8-hydroxyquinolinium 4-nitrobenzoate:4-nitrobenzoic acid, sulfaproxyline:caffeine, retro-inverso-isopropyl (2R,3S)-4-cyclohexyl-2-hydroxy-3-(N-((2R)-2-morpholinocarbonylmethyl-3-(1-naphthyl)propionyl)-L-histidylamino)butyrate:cinnamic acid monohydrate, benzoic acid:isonicotinamide, 3-(2-N′,N′-(dimethylhydrazino)-4-thiazolylmethylthio)-N″-sulfamoylpropionamidine:maleic acid, diglycine hydrochloride (C₂H₅NO₂:C₂H₆NO₂⁺Cl⁻), octadecanoic acid:3-pyridinecarboxamide, cis-N-(3-methyl-1-(2-(1,2,3,4-tetrahydro)naphthyl)-piperidin-4-yl)-N-phenylpropanamide hydrochloride:oxalic acid, trans-N-(3-methyl-1-(2-(1,2,3,4-tetrahydro)naphthyl)-piperidin-4-ylium)-N-phenylpropanamide oxalate:oxalic acid dihydrate, bis(1-(3-((4-(2-isopropoxyphenyl)-1-piperazinyl)methyl)benzoyl)piperidine) succinate:succinic acid, bis(p-cyanophenyl)imidazolylmethane:succinic acid, cis-1-((4-(1-imidazolylmethyl)cyclohexyl)methyl)imidazole:succinic acid, (+)-2-(5,6-dimethoxy-1,2,3,4-tetrahydro-1-naphthyl)imidazoline:(+)-dibenzoyl-D-tartaric acid, raclopride:tartaric acid, 2,6-diamino-9-ethylpurine:5,5-diethylbarbituric acid, 5,5-diethylbarbituric acid:bis(2-aminopyridine), 5,5-diethylbarbituric acid:acetamide, 5,5-diethylbarbituric acid:KI₃5,5-diethylbarbituric acid:urea, bis(barbital):hexamethylphosphoramide, 5,5-diethylbarbituric acid:imidazole, barbital:1-methylimidazole, 5,5-diethylbarbituric acid:N-methyl-2-pyridone, 2,4-diamino-5-(3,4,5-trimethoxybenzyl)-pyrimidine:5,5-diethylbarbituric acid, bis(barbital):caffeine, bis(barbital):1-methylimidazole, bis(beta-cyclodextrin):bis(barbital) hydrate, tetrakis(beta-cyclodextrin):tetrakis(barbital), 9-ethyladenine:5,5-diethylbarbituric acid, barbital:N′-(p-cyanophenyl)-N-(p-iodophenyl)melamine, barbital:2-amino-4-(m-bromophenylamino)-6-chloro-1,3,5-triazine, 5,5-diethylbarbituric acid:N,N′-diphenylmelamine, 5,5-diethylbarbituric acid:N,N′-bis(p-chlorophenyl)melamine, N,N′-bis(p-bromophenyl)melamine:5,5-diethylbarbituric acid, 5,5-diethylbarbituric acid:N,N′-bis(p-iodophenyl)melamine, 5,5-diethylbarbituric acid:N,N′-bis(p-tolyl)melamine, 5,5-diethylbarbituric acid:N,N′-bis(m-tolyl)melamine, 5,5-diethylbarbituric acid:N,N′-bis(m-chlorophenyl)melamine, N,N′-Bis(m-methylphenyl)melamine:barbital, N,N′-bis(m-chlorophenyl)melamine:barbital tetrahydrofuran solvate, 5,5-diethylbarbituric acid:N,N′-bis(tert-butyl)melamine, 5,5-diethylbarbituric acid:N,N′-di(tert-butyl)melamine, 6,6′-diquinolyl ether:5,5-diethylbarbituric acid, 5-tert-butyl-2,4,6-triaminopyrimidine:diethylbarbituric acid, N,N′-bis(4-carboxymethylphenyl)melamine:barbital ethanol solvate, N,N′-bis(4-tent-butylphenyl)melamine:barbital, tris(5,17-N,N′-bis(4-amino-6-(butylamino)-1,3,5-triazin-2-yl)diamino-11,23-dinitro-25,26,27,28-tetrapropoxycalix(4)arene):hexakis(diethylbarbituric acid) toluene solvate, N,N′-bis(m-fluorophenyl)melamine:barbital, N,N′-bis(m-bromophenyl)melamine:barbital acetone solvate, N,N′-bis(m-iodophenyl)melamine:barbital acetonitrile solvate, N,N′-bis(m-trifluoromethylphenyl)melamine:barbital acetonitrile solvate, aminopyrine:barbital, N,N′-bis(4-fluorophenyl)melamine:barbital, N,N′-bis(4-trifluoromethylphenyl)melamine:barbital, 2,4-diamino-5-(3,4,5-trimethoxybenzyl)pyrimidine:barbital, hydroxybutyrate:hydroxyvalerate, 2-aminopyrimidine:succinic acid, 1,3-bis(((6-methylpyrid-2-yl)amino)carbonyl)benzene:glutaric acid, 5-tert-butyl-2,4,6-triaminopyrimidine:diethylbarbituric acid, bis(dithiobiuret-S,S′)nickel(II):diuracil, platinum 3,3′-dihydroxymethyl-2,2′-bipyridine dichloride:AgF₃CSO₃, 4,4′-bipyridyl:isophthalic acid, 4,4′-bipyridyl:1,4-naphthalenedicarboxylic acid, 4,4′-bipyridyl:1,3,5-cyclohexane-tricarboxylic acid, 4,4′-bipyridyl:tricaballylic acid, urotropin:azelaic acid, insulin:C8-HI (octanoyl-N^{e}-LysB29-human insulin), isonicotinamide:cinnamic acid, isonicotinamide:3-hydroxybenzoic acid, isonicotinamide:3-N,N-dimethylaminobenzoic acid, isonicotinamide:3,5-bis(trifluoromethyl)-benzoic acid, isonicotinamide:d,l-mandelic acid, isonicotinamide:chloroacetic acid, isonicotinamide:fumaric acid monoethyl ester, isonicotinamide:12-bromododecanoic acid, isonicotinamide:fumaric acid, isonicotinamide:succinic acid, isonicotinamide:4-ketopimelic acid, isonicotinamide:thiodiglycolic acid, 1,3,5-cyclohexane-tricarboxylic acid:hexamethyltetramine, 1,3,5-cyclohexane-tricarboxylic acid:4,7-phenanthroline, 4,7-phenanthroline:oxalic acid, 4,7-phenanthroline:terephthalic acid, 4,7-phenanthroline: 1,3,5-cyclohexane-tricarboxylic acid, 4,7-phenanthroline:1,4-naphthalenedicarboxylic acid, pyrazine:methanoic acid, pyrazine:ethanoic acid, pyrazine:propanoic acid, pyrazine:butanoic acid, pyrazine:pentanoic acid, pyrazine:hexanoic acid, pyrazine:heptanoic acid, pyrazine:octanoic acid, pyrazine:nonanoic acid, pyrazine:decanoic acid, diammine-(deoxy-quanyl-quanyl-N⁷,N⁷)-platinum:tris(glycine) hydrate, 2-aminopyrimidine:p-phenylenediacetic acid, bis(2-aminopyrimidin-1-ium)fumarate:fumaric acid, 2-aminopyrimidine:indole-3-acetic acid, 2-aminopyrimidine:N-methylpyrrole-2-carboxylic acid, 2-aminopyrimidine:thiophen-2-carboxylic acid, 2-aminopyrimidine:(+)-camphoric acid, 2,4,6-Trinitrobenzoic acid:2-aminopyrimidine, 2-aminopyrimidine:4-aminobenzoic acid, 2-aminopyrimidine:bis(phenoxyacetic acid), 2-aminopyrimidine:(2,4-dichlorophenoxy)acetic acid, 2-aminopyrimidine:(3,4-dichlorophenoxy)acetic acid, 2-aminopyrimidine:indole-2-carboxylic acid, 2-aminopyrimidine:terephthalic acid, 2-aminopyrimidine:bis(2-nitrobenzoic acid), 2-aminopyrimidine:bis(2-aminobenzoic acid), 2-aminopyrimidine:3-aminobenzoic acid, 2-hexeneoic acid:isonicotinamide, 4-nitrobenzoic acid:isonicotinamide, 3,5-dinitrobenzoic acid:isonicotinamide:4-methylbenzoic acid, 2-amino-5-nitropyrimidine:2-amino-3-nitropyridine, 3,5-dinitrobenzoic acid:4-chlorobenzamide, 3-dimethylaminobenzoic acid:4-chlorobenzamide, fumaric acid:4-chlorobenzamide, oxine:4-nitrobenzoic acid, oxine:3,5-dinitrobenzoic acid, oxine:3,5-dinitrosalicylic acid, 3-[2-(N′,N′-dimethylhydrazino)-4-thiazolylmethylthio]-N²-sulfamoylpropionamidine:maleic acid, 5-fluorouracil:9-ethylhypoxanthine, 5-fluorouracil:cytosine dihydrate, 5-fluorouracil:theophylline monohydrate, stearic acid:nicotinamide, cis-1-{[4-(1-imidazolylmethyl)cyclohexyl]methyl}imidazole:succinic acid, CGS18320B:succinic acid, sulfaproxyline:caffeine, 4-aminobenzoic acid:4-aminobenzonitrile, 3,5-dinitrobenzoic acid:isonicotinamide:3-methylbenzoic acid, 3,5-dinitrobenzoic acid:isonicotinamide:4-(dimethylamino)benzoic acid, 3,5-dinitrobenzoic acid:isonicotinamide:4-hydroxy-3-methoxycinnamic acid, isonicotinamide:oxalic acid, isonicotinamide:malonic acid, isonicotinamide:succinic acid, isonicotinamide:glutaric acid, isonicotinamide:adipic acid, benzoic acid:isonicotinamide, mazapertine:succinate, betaine:dichloronitrophenol, betainepyridine:dichloronitrophenol, betainepyridine:pentachlorophenol, 4-{2-[1-(2-hydroxyethyl)-4-pyridylidene]-ethylidene}-cyclo-hexa-2,5-dien-1-one:methyl 2,4-dihydroxybenzoate, 4-{2-[1-(2-hydroxyethyl)-4-pyridylidene]-ethylidene}-cyclo-hexa-2,5-dien-1-one:2,4-dihydroxypropiophenone, 4-{2-[1-(2-hydroxyethyl)-4-pyridylidene]-ethylidene}-cyclo-hexa-2,5-dien-1-one:2,4-dihydroxyacetophenone, squaric acid:4,4′-dipyridylacetylene, squaric acid:1,2-bis(4-pyridyl)ethylene, chloranilic acid:1,4-bis[(4-pyridyl)ethynyl]benzene, 4,4′-bipyridine:phthalic acid, 4,4′-dipyridylacetylene:phthalic acid, bis(pentamethylcyclopentadienyl)iron:bromanilic acid, bis(pentamethylcyclopentadienyl)iron:chloranilic acid, bis(pentamethylcyclopentadienyl)iron:cyananilic acid, pyrazinotetrathiafulvalene:chloranilic acid, phenol:pentafluorophenol, co-crystals of cis-itraconazole, and co-crystals of topiramate are specifically excluded from the present invention.

In another embodiment, a pharmaceutical composition can be formulated to contain an API in co-crystal form as micronized or nano-sized particles. More specifically, another embodiment couples the processing of a pure API to a co-crystal form with the process of making a controlled particle size for manipulation into a pharmaceutical dosage form. This embodiment combines two processing steps into a single step via techniques such as, but not limited to, grinding, alloying, or sintering (i.e., heating a powder mix). The coupling of these processes overcomes a serious limitation of having to isolate and store the bulk drug that is required for a formulation, which in some cases can be difficult to isolate (e.g., amorphous, chemically or physically unstable).

Excipients employed in pharmaceutical compositions of the present invention can be solids, semi-solids, liquids or combinations thereof. Preferably, excipients are solids. Compositions of the invention containing excipients can be prepared by any known technique of pharmacy that comprises admixing an excipient with an API or therapeutic agent. A pharmaceutical composition of the invention contains a desired amount of API per dose unit and, if intended for oral administration, can be in the form, for example, of a tablet, a caplet, a pill, a hard or soft capsule, a lozenge, a cachet, a dispensable powder, granules, a suspension, an elixir, a dispersion, or any other form reasonably adapted for such administration. If intended for parenteral administration, it can be in the form, for example, of a suspension or transdermal patch. If intended for rectal administration, it can be in the form, for example, of a suppository. Presently preferred are oral dosage forms that are discrete dose units each containing a predetermined amount of the API, such as tablets or capsules.

In another embodiment, APIs with an inappropriate pH for transdermal patches can be co-crystallized with an appropriate co-crystal former, thereby adjusting its pH to an appropriate level for use as a transdermal patch. In another embodiment, an APIs pH level can be optimized for use in a transdermal patch via co-crystallization with an appropriate co-crystal former.

Non-limiting examples follow of excipients that can be used to prepare pharmaceutical compositions of the invention.

Pharmaceutical compositions of the invention optionally comprise one or more pharmaceutically acceptable carriers or diluents as excipients. Suitable carriers or diluents illustratively include, but are not limited to, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of alpha- and amorphous cellulose (e.g., RexcelJ), powdered cellulose, hydroxypropylcellulose (HPC) and hydroxypropylmethylcellulose (HPMC); calcium carbonate; glycine; bentonite; block co-polymers; polyvinylpyrrolidone; and the like. Such carriers or diluents, if present, constitute in total about 5% to about 99%, preferably about 10% to about 85%, and more preferably about 20% to about 80%, of the total weight of the composition. The carrier, carriers, diluent, or diluents selected preferably exhibit suitable flow properties and, where tablets are desired, compressibility.

Lactose, mannitol, dibasic sodium phosphate, and microcrystalline cellulose (particularly Avicel PH microcrystalline cellulose such as Avicel PH 101), either individually or in combination, are preferred diluents. These diluents are chemically compatible with many co-crystals described herein. The use of extragranular microcrystalline cellulose (that is, microcrystalline cellulose added to a granulated composition) can be used to improve hardness (for tablets) and/or disintegration time. Lactose, especially lactose monohydrate, is particularly preferred. Lactose typically provides compositions having suitable release rates of co-crystals, stability, pre-compression flowability, and/or drying properties at a relatively low diluent cost. It provides a high density substrate that aids densification during granulation (where wet granulation is employed) and therefore improves blend flow properties and tablet properties.

Pharmaceutical compositions of the invention optionally comprise one or more pharmaceutically acceptable disintegrants as excipients, particularly for tablet formulations. Suitable disintegrants include, but are not limited to, either individually or in combination, starches, including sodium starch glycolate (e.g., Explotab™ of PenWest) and pregelatinized corn starches (e.g., National™ 1551 of National Starch and Chemical Company, National™ 1550, and Colorcon™ 1500), clays (e.g., Veegum™ HV of R.T. Vanderbilt), celluloses such as purified cellulose, microcrystalline cellulose, methylcellulose, carboxymethylcellulose and sodium carboxymethylcellulose, croscarmellose sodium (e.g., Ac-Di-Sol™ of FMC), alginates, crospovidone, and gums such as agar, guar, locust bean, karaya, pectin and tragacanth gums.

Disintegrants may be added at any suitable step during the preparation of the composition, particularly prior to granulation or during a lubrication step prior to compression. Such disintegrants, if present, constitute in total about 0.2% to about 30%, preferably about 0.2% to about 10%, and more preferably about 0.2% to about 5%, of the total weight of the composition.

Croscarmellose sodium is a preferred disintegrant for tablet or capsule disintegration, and, if present, preferably constitutes about 0.2% to about 10%, more preferably about 0.2% to about 7%, and still more preferably about 0.2% to about 5%, of the total weight of the composition. Croscarmellose sodium confers superior intragranular disintegration capabilities to granulated pharmaceutical compositions of the present invention.

Pharmaceutical compositions of the invention optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Such binding agents may also prevent or inhibit crystallization or recrystallization of a co-crystal of the present invention once the salt has been dissolved in a solution. Suitable binding agents and adhesives include, but are not limited to, either individually or in combination, acacia; tragacanth; sucrose; gelatin; glucose; starches such as, but not limited to, pregelatinized starches (e.g., National™ 1511 and National™ 1500); celluloses such as, but not limited to, methylcellulose and carmellose sodium (e.g., Tylose™); alginic acid and salts of alginic acid; magnesium aluminum silicate; PEG; guar gum; polysaccharide acids; bentonites; povidone, for example povidone K-15, K-30 and K-29/32; polymethacrylates; HPMC; hydroxypropylcellulose (e.g., Klucel™ of Aqualon); and ethylcellulose (e.g., Ethocel™ of the Dow Chemical Company). Such binding agents and/or adhesives, if present, constitute in total about 0.5% to about 25%, preferably about 0.75% to about 15%, and more preferably about 1% to about 10%, of the total weight of the pharmaceutical composition.

Many of the binding agents are polymers comprising amide, ester, ether, alcohol or ketone groups and, as such, are preferably included in pharmaceutical compositions of the present invention. Polyvinylpyrrolidones such as povidone K-30 are especially preferred. Polymeric binding agents can have varying molecular weight, degrees of crosslinking, and grades of polymer. Polymeric binding agents can also be copolymers, such as block co-polymers that contain mixtures of ethylene oxide and propylene oxide units. Variation in these units' ratios in a given polymer affects properties and performance. Examples of block co-polymers with varying compositions of block units are Poloxamer 188 and Poloxamer 237 (BASF Corporation).

Pharmaceutical compositions of the invention optionally comprise one or more pharmaceutically acceptable wetting agents as excipients. Such wetting agents are preferably selected to maintain the co-crystal in close association with water, a condition that is believed to improve bioavailability of the composition. Such wetting agents can also be useful in solubilizing or increasing the solubility of co-crystals.

Non-limiting examples of surfactants that can be used as wetting agents in pharmaceutical compositions of the invention include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and degrees Ctoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefosse), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefosse), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, preferably about 0.4% to about 10%, and more preferably about 0.5% to about 5%, of the total weight of the pharmaceutical composition.

Wetting agents that are anionic surfactants are preferred. Sodium lauryl sulfate is a particularly preferred wetting agent. Sodium lauryl sulfate, if present, constitutes about 0.25% to about 7%, more preferably about 0.4% to about 4%, and still more preferably about 0.5% to about 2%, of the total weight of the pharmaceutical composition.

Pharmaceutical compositions of the invention optionally comprise one or more pharmaceutically acceptable lubricants (including anti-adherents and/or glidants) as excipients. Suitable lubricants include, but are not limited to, either individually or in combination, glyceryl behapate (e.g., Compritol™ 888 of Gattefosse); stearic acid and salts thereof, including magnesium, calcium and sodium stearates; hydrogenated vegetable oils (e.g., Sterotex™ of Abitec); colloidal silica; talc; waxes; boric acid; sodium benzoate; sodium acetate; sodium fumarate; sodium chloride; DL-leucine; PEG (e.g., Carbowax™ 4000 and Carbowax™ 6000 of the Dow Chemical Company); sodium oleate; sodium lauryl sulfate; and magnesium lauryl sulfate. Such lubricants, if present, constitute in total about 0.1% to about 10%, preferably about 0.2% to about 8%, and more preferably about 0.25% to about 5%, of the total weight of the pharmaceutical composition.

Magnesium stearate is a preferred lubricant used, for example, to reduce friction between the equipment and granulated mixture during compression of tablet formulations.

Suitable anti-adherents include, but are not limited to, talc, cornstarch, DL-leucine, sodium lauryl sulfate and metallic stearates. Talc is a preferred anti-adherent or glidant used, for example, to reduce formulation sticking to equipment surfaces and also to reduce static in the blend. Talc, if present, constitutes about 0.1% to about 10%, more preferably about 0.25% to about 5%, and still more preferably about 0.5% to about 2%, of the total weight of the pharmaceutical composition.

Glidants can be used to promote powder flow of a solid formulation. Suitable glidants include, but are not limited to, colloidal silicon dioxide, starch, talc, tribasic calcium phosphate, powdered cellulose and magnesium trisilicate. Colloidal silicon dioxide is particularly preferred.

Other excipients such as colorants, flavors and sweeteners are known in the pharmaceutical art and can be used in pharmaceutical compositions of the present invention. Tablets can be coated, for example with an enteric coating, or uncoated. Compositions of the invention can further comprise, for example, buffering agents.

Optionally, one or more effervescent agents can be used as disintegrants and/or to enhance organoleptic properties of pharmaceutical compositions of the invention. When present in pharmaceutical compositions of the invention to promote dosage form disintegration, one or more effervescent agents are preferably present in a total amount of about 30% to about 75%, and preferably about 45% to about 70%, for example about 60%, by weight of the pharmaceutical composition.

According to a particularly preferred embodiment of the invention, an effervescent agent, present in a solid dosage form in an amount less than that effective to promote disintegration of the dosage form, provides improved dispersion of the API in an aqueous medium. Without being bound by theory, it is believed that the effervescent agent is effective to accelerate dispersion of the API from the dosage form in the gastrointestinal tract, thereby further enhancing absorption and rapid onset of therapeutic effect. When present in a pharmaceutical composition of the invention to promote intragastrointestinal dispersion but not to enhance disintegration, an effervescent agent is preferably present in an amount of about 1% to about 20%, more preferably about 2.5% to about 15%, and still more preferably about 5% to about 10%, by weight of the pharmaceutical composition.

An “effervescent agent” herein is an agent comprising one or more compounds which, acting together or individually, evolve a gas on contact with water. The gas evolved is generally oxygen or, most commonly, carbon dioxide. Preferred effervescent agents comprise an acid and a base that react in the presence of water to generate carbon dioxide gas. Preferably, the base comprises an alkali metal or alkaline earth metal carbonate or bicarbonate and the acid comprises an aliphatic carboxylic acid.

Non-limiting examples of suitable bases as components of effervescent agents useful in the invention include carbonate salts (e.g., calcium carbonate), bicarbonate salts (e.g., sodium bicarbonate), sesquicarbonate salts, and mixtures thereof. Calcium carbonate is a preferred base.

Non-limiting examples of suitable acids as components of effervescent agents and/or solid organic acids useful in the invention include citric acid, tartaric acid (as D-, L-, or D/L-tartaric acid), malic acid (as D-, L-, or DL-malic acid), maleic acid, fumaric acid, adipic acid, succinic acid, acid anhydrides of such acids, acid salts of such acids, and mixtures thereof. Citric acid is a preferred acid.

In a preferred embodiment of the invention, where the effervescent agent comprises an acid and a base, the weight ratio of the acid to the base is about 1:100 to about 100:1, more preferably about 1:50 to about 50:1, and still more preferably about 1:10 to about 10:1. In a further preferred embodiment of the invention, where the effervescent agent comprises an acid and a base, the ratio of the acid to the base is approximately stoichiometric.

Excipients which solubilize APIs typically have both hydrophilic and hydrophobic regions, or are preferably amphiphilic or have amphiphilic regions. One type of amphiphilic or partially-amphiphilic excipient comprises an amphiphilic polymer or is an amphiphilic polymer. A specific amphiphilic polymer is a polyalkylene glycol, which is commonly comprised of ethylene glycol and/or propylene glycol subunits. Such polyalkylene glycols can be esterified at their termini by a carboxylic acid, ester, acid anhydride or other suitable moiety. Examples of such excipients include poloxamers (symmetric block copolymers of ethylene glycol and propylene glycol; e.g., poloxamer 237), polyalkyene glycolated esters of tocopherol (including esters formed from a di- or multi-functional carboxylic acid; e.g., d-alpha-tocopherol polyethylene glycol-1000 succinate), and macrogolglycerides (formed by alcoholysis of an oil and esterification of a polyalkylene glycol to produce a mixture of mono-, di- and tri-glycerides and mono- and di-esters; e.g., stearoyl macrogol-32 glycerides). Such pharmaceutical compositions are advantageously administered orally.

Pharmaceutical compositions of the present invention can comprise about 10% to about 50%, about 25% to about 50%, about 30% to about 45%, or about 30% to about 35% by weight of a co-crystal; about 10% to about 50%, about 25% to about 50%, about 30% to about 45%, or about 30% to about 35% by weight of an excipient which inhibits crystallization in aqueous solution, in simulated gastric fluid, or in simulated intestinal fluid; and about 5% to about 50%, about 10% to about 40%, about 15% to about 35%, or about 30% to about 35% by weight of a binding agent. In one example, the weight ratio of the co-crystal to the excipient which inhibits crystallization to binding agent is about 1 to 1 to 1.

Solid dosage forms of the invention can be prepared by any suitable process, not limited to processes described herein.

An illustrative process comprises (a) a step of blending an API of the invention with one or more excipients to form a blend, and (b) a step of tableting or encapsulating the blend to form tablets or capsules, respectively.

In a preferred process, solid dosage forms are prepared by a process comprising (a) a step of blending a co-crystal of the invention with one or more excipients to form a blend, (b) a step of granulating the blend to form a granulate, and (c) a step of tableting or encapsulating the blend to form tablets or capsules respectively. Step (b) can be accomplished by any dry or wet granulation technique known in the art, but is preferably a dry granulation step. A salt of the present invention is advantageously granulated to form particles of about 1 micrometer to about 100 micrometer, about 5 micrometer to about 50 micrometer, or about 10 micrometer to about 25 micrometer. One or more diluents, one or more disintegrants and one or more binding agents are preferably added, for example in the blending step, a wetting agent can optionally be added, for example in the granulating step, and one or more disintegrants are preferably added after granulating but before tableting or encapsulating. A lubricant is preferably added before tableting. Blending and granulating can be performed independently under low or high shear. A process is preferably selected that forms a granulate that is uniform in API content, that readily disintegrates, that flows with sufficient ease so that weight variation can be reliably controlled during capsule filling or tableting, and that is dense enough in bulk so that a batch can be processed in the selected equipment and individual doses fit into the specified capsules or tablet dies.

In an alternative embodiment, solid dosage forms are prepared by a process that includes a spray drying step, wherein an API is suspended with one or more excipients in one or more sprayable liquids, preferably a non-protic (e.g., non-aqueous or non-alcoholic) sprayable liquid, and then is rapidly spray dried over a current of warm air. A granulate or spray dried powder resulting from any of the above illustrative processes can be compressed or molded to prepare tablets or encapsulated to prepare capsules. Conventional tableting and encapsulation techniques known in the art can be employed. Where coated tablets are desired, conventional coating techniques are suitable.

Excipients for tablet compositions of the invention are preferably selected to provide a disintegration time of less than about 30 minutes, preferably about 25 minutes or less, more preferably about 20 minutes or less, and still more preferably about 15 minutes or less, in a standard disintegration assay.

Pharmaceutically acceptable co-crystals can be administered by controlled-, sustained-, or delayed-release means. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 Technomic Publishing, Lancaster, Pa.: 2000).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under dosing a drug (i.e., going below the minimum therapeutic levels) as well as exceeding the toxicity level for the drug.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the co-crystals and compositions of the invention. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed co-crystals and thus effect controlled delivery of the drug. Examples of specific anion exchangers include, but are not limited to, Duolite® A568 and Duolite® AP143 (Rohm & Haas, Spring House, Pa. USA).

One embodiment of the invention encompasses a unit dosage form which comprises a pharmaceutically acceptable co-crystal, or a solvate, hydrate, dehydrate, anhydrous, or amorphous form thereof, and one or more pharmaceutically acceptable excipients or diluents, wherein the pharmaceutical composition or dosage form is formulated for controlled-release. Specific dosage forms utilize an osmotic drug delivery system.

A particular and well-known osmotic drug delivery system is referred to as OROS® (Alza Corporation, Mountain View, Calif. USA). This technology can readily be adapted for the delivery of compounds and compositions of the invention. Various aspects of the technology are disclosed in U.S. Pat. Nos. 6,375,978 B1; 6,368,626 B1; 6,342,249 B1; 6,333,050 B2; 6,287,295 B1; 6,283,953 B1; 6,270,787 B1; 6,245,357 B1; and 6,132,420; each of which is incorporated herein by reference. Specific adaptations of OROS® that can be used to administer compounds and compositions of the invention include, but are not limited to, the OROS® Push-Pull™, Delayed Push-Pull™, Multi-Layer Push-Pull™, and Push-Stick™ Systems, all of which are well known. See, e.g., http://www.alza.com. Additional OROS® systems that can be used for the controlled oral delivery of compounds and compositions of the invention include OROS®-CT and L-OROS®. Id.; see also, Delivery Times, vol. II, issue II (Alza Corporation).

Conventional OROS® oral dosage forms are made by compressing a drug powder (e.g. co-crystal) into a hard tablet, coating the tablet with cellulose derivatives to form a semi-permeable membrane, and then drilling an orifice in the coating (e.g., with a laser). Kim, Cherng-ju, Controlled Release Dosage Form Design, 231-238 (Technomic Publishing, Lancaster, Pa.: 2000). The advantage of such dosage forms is that the delivery rate of the drug is not influenced by physiological or experimental conditions. Even a drug with a pH-dependent solubility can be delivered at a constant rate regardless of the pH of the delivery medium. But because these advantages are provided by a build-up of osmotic pressure within the dosage form after administration, conventional OROS® drug delivery systems cannot be used to effectively deliver drugs with low water solubility. Id. at 234. Because co-crystals of this invention can be far more soluble in water than the API itself, they are well suited for osmotic-based delivery to patients. This invention does, however, encompass the incorporation of conventional crystalline API (e.g. pure API without co-crystal former), and non-salt isomers and isomeric mixtures thereof, into OROS® dosage forms.

A specific dosage form of the invention comprises: a wall defining a cavity, the wall having an exit orifice formed or formable therein and at least a portion of the wall being semipermeable; an expandable layer located within the cavity remote from the exit orifice and in fluid communication with the semipermeable portion of the wall; a dry or substantially dry state drug layer located within the cavity adjacent to the exit orifice and in direct or indirect contacting relationship with the expandable layer; and a flow-promoting layer interposed between the inner surface of the wall and at least the external surface of the drug layer located within the cavity, wherein the drug layer comprises a co-crystal, or a solvate, hydrate, dehydrate, anhydrous, or amorphous form thereof. See U.S. Pat. No. 6,368,626, the entirety of which is incorporated herein by reference.

Another specific dosage form of the invention comprises: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a liquid, active agent formulation absorbed in porous particles, the porous particles being adapted to resist compaction forces sufficient to form a compacted drug layer without significant exudation of the liquid, active agent formulation, the dosage form optionally having a placebo layer between the exit orifice and the drug layer, wherein the active agent formulation comprises a co-crystal, or a solvate, hydrate, dehydrate, anhydrous, or amorphous form thereof. See U.S. Pat. No. 6,342,249, the entirety of which is incorporated herein by reference.

The invention will now be described in further detail, by way of example, with reference to the accompanying drawings.

### EXEMPLIFICATION

General Methods for the Preparation of Co-Crystals

a) High Throughput Crystallization Using the CrystalMax™ Platform

CrystalMax™ comprises a sequence of automated, integrated high throughput robotic stations capable of rapid generation, identification and characterization of polymorphs, salts, and co-crystals of APIs and API candidates. Worksheet generation and combinatorial mixture design is carried out using proprietary design software Architect™. Typically, an API or an API candidate is dispensed from an organic solvent into tubes and dried under a stream of nitrogen. Salts and/or co-crystal formers may also be dispensed and dried in the same fashion. Water and organic solvents may be combinatorially dispensed into the tubes using a multi-channel dispenser. Each tube in a 96-tube array is then sealed within 15 seconds of combinatorial dispensing to avoid solvent evaporation. The mixtures are then rendered supersaturated by heating to 70 degrees C. for 2 hours followed by a 1 degree C./minute cooling ramp to 5 degrees C. Optical checks are then conducted to detect crystals and/or solid material. Once a solid has been identified in a tube, it is isolated through aspiration and drying. Raman spectra are then obtained on the solids and cluster classification of the spectral patterns is performed using proprietary software (Inquire™).

b) Crystallization from Solution

Co-crystals may be obtained by dissolving the separate components in a solvent and adding one to the other. The co-crystal may then precipitate or crystallize as the solvent mixture is evaporated slowly. The co-crystal may also be obtained by dissolving the two components in the same solvent or a mixture of solvents.

c) Crystallization from the Melt (Co-Melting)

A co-crystal may be obtained by melting the two components together (i.e., co-melting) and allowing recrystallization to occur. In some cases, an anti-solvent may be added to facilitate crystallization.

d) Thermal Microscopy

A co-crystal may be obtained by melting the higher melting component on a glass slide and allowing it to recrystallize. The second component is then melted and is also allowed to recrystallize. The co-crystal may form as a separated phase/band in between the eutectic bands of the two original components.

e) Mixing and/or Grinding

A co-crystal may be obtained by mixing or grinding two components together in the solid state.

f) Co-Sublimation

A co-crystal may be obtained by co-subliming a mixture of an API and a co-crystal former in the same sample cell as an intimate mixture either by heating, mixing or placing the mixture under vacuum. A co-crystal may also be obtained by co-sublimation using a Kneudsen apparatus where the API and the co-crystal former are contained in separate sample cells, connected to a single cold finger, each of the sample cells is maintained at the same or different temperatures under a vacuum atmosphere in order to co-sublime the two components onto the cold-finger forming the desired co-crystal.

### Analytical Methods

### Procedure for DSC Analysis

DSC analysis of the samples was performed using a Q1000 Differential Scanning Calorimeter (TA Instruments, New Castle, Del., U.S.A.), which uses Advantage for QW-Series, version 1.0.0.78, Thermal Advantage Release 2.0 (2001 TA Instruments-Water LLC). In addition, the analysis software used was Universal. Analysis 2000 for Windows 95/95/2000/NT, version 3.1E; Build 3.1.0.40 (2001 TA Instruments-Water LLC).

For the DSC analysis, the purge gas used was dry nitrogen, the reference material was an empty aluminum pan that was crimped, and the sample purge was 50 mL/minute.

DSC analysis of the sample was performed by placing ≦2 mg of sample in an aluminum pan with a crimped pan closure. The starting temperature was typically 20 degrees C. with a heating rate of 10 degrees C./minute, and the ending temperature was 300 degrees C. Unless otherwise indicated, all reported transitions are as stated +/−1.0 degrees C.

### Procedure for TGA Analysis

TGA analysis of samples was performed using a Q500 Thermogravimetric Analyzer (TA Instruments, New Castle, Del., U.S.A.), which uses Advantage for QW-Series, version 1.0.0.78, Thermal Advantage Release 2.0 (2001 TA Instruments-Water LLC). In addition, the analysis software used was Universal Analysis 2000 for Windows 95/95/2000/NT, version 3.1E; Build 3.1.0.40 (2001 TA Instruments-Water LLC).

For all of the TGA experiments, the purge gas used was dry nitrogen, the balance purge was 40 mL/minute N₂, and the sample purge was 60 mL/minute N₂.

TGA of the sample was performed by placing 2 mg of sample in a platinum pan. The starting temperature was typically 20 degrees C. with a heating rate of 10 degrees C./minute, and the ending temperature was 300 degrees C.

### Procedure for PXRD Analysis

A powder X-ray diffraction pattern for the samples was obtained using a D/Max Rapid, Contact (Rigaku/MSC, The Woodlands, Tex., U.S.A.), which uses as its control software RINT Rapid Control software, Rigaku Rapid/XRD, version 1.0.0 (1999 Rigaku Co.). In addition, the analysis software used were RINT Rapid display software, version 1.18 (Rigaku/MSC), and JADE XRD Pattern Processing, versions 5.0 and 6.0 ((1995-2002, Materials Data, Inc.).

For the PXRD analysis, the acquisition parameters were as follows: source was Cu with a K line at 1.5406 Å; x-y stage was manual; collimator size was 0.3 or 0.8 mm; capillary tube (Charles Supper Company, Natick, Mass., U.S.A.) was 0.3 mm ID; reflection mode was used; the power to the X-ray tube was 46 kV; the current to the X-ray tube was 40 mA; the omega-axis was oscillating in a range of 0-5 degrees at a speed of 1 degree/minute; the phi-axis was spinning at an angle of 360 degrees at a speed of 2 degrees/second; 0.3 or 0.8 mm collimator; the collection time was 60 minutes; the temperature was room temperature; and the heater was not used. The sample was presented to the X-ray source in a boron rich glass capillary.

In addition, the analysis parameters were as follows: the integration 2-theta range was 2-40 or 60 degrees; the integration chi range was 0-360 degrees; the number of chi segments was 1; the step size used was 0.02; the integration utility was cylint; normalization was used; dark counts were 8; omega offset was 180; and chi and phi offsets were 0.

The relative intensity of peaks in a diffractogram is not necessarily a limitation of the PXRD pattern because peak intensity can vary from sample to sample, e.g., due to crystalline impurities. Further, the angles of each peak can vary by about +/−0.1 degrees, preferably +/−0.05. The entire pattern or most of the pattern peaks may also shift by about +/−0.1 degree due to differences in calibration, settings, and other variations from instrument to instrument and from operator to operator.

### Procedure for Raman Acquisition, Filtering and Binning

### Acquisition

The sample was either left in the glass vial in which it was processed or an aliquot of the sample was transferred to a glass slide. The glass vial or slide was positioned in the sample chamber. The measurement was made using an Almega™ Dispersive Raman (Almega™ Dispersive Raman, Thermo-Nicolet, 5225 Verona Road, Madison, Wis. 53711-4495) system fitted with a 785 nm laser source. The sample was manually brought into focus using the microscope portion of the apparatus with a 10× power objective (unless otherwise noted), thus directing the laser onto the surface of the sample. The spectrum was acquired using the parameters outlined in Table XXII. (Exposure times and number of exposures may vary; changes to parameters will be indicated for each acquisition.)

### Filtering and Binning

Each spectrum in a set was filtered using a matched filter of feature size 25 to remove background signals, including glass contributions and sample fluorescence. This is particularly important as large background signal or fluorescence limit the ability to accurately pick and assign peak positions in the subsequent steps of the binning process. Filtered spectra were binned using the peak pick and bin algorithm with the parameters given in Table XXIII. The sorted cluster diagrams for each sample set and the corresponding cluster assignments for each spectral file were used to identify groups of samples with similar spectra, which was used to identify samples for secondary analyses.

TABLE XXII
Raman Spectral acquisition parameters

| Parameter | Setting Used |
|---|---|
| Exposure time (s) | 2.0 |
| Number of exposures | 10 |
| Laser source wavelength (nm) | 785 |
| Laser power (%) | 100 |
| Aperture shape | pin hole |
| Aperture size (um) | 100 |
| Spectral range | 104-3428 |
| Grating position | Single |
| Temperature at acquisition (degrees C.) | 24.0 |

TABLE XXIII
Raman Filtering and Binning Parameters

| Parameter | Setting Used |
|---|---|
| Filtering Parameters | |
| Filter type | Matched |
| Filter size | 25 |
| QC Parameters | |
| Peak Height Threshold | 1000 |
| Region for noise test (cm⁻¹) | 0-10000 |
| RMS noise threshold | 10000 |
| Automatically eliminate failed | Yes |
| spectra | |
| Region of Interest | |
| Include (cm⁻¹) | 104-3428 |
| Exclude region I (cm⁻¹) | |
| Exclude region II (cm⁻¹) | |
| Exclude region III (cm⁻¹) | |
| Exclude region IV (cm⁻¹) | |
| Peak Pick Parameters | |
| Peak Pick Sensitivity | Variable |
| Peak Pick Threshold | 100 |
| Peak Comparison Parameters | |
| Peak Window (cm⁻¹) | 2 |
| Analysis Parameters | |
| Number of clusters | Variable |

### Procedure for Single Crystal X-Ray Diffraction

Single crystal x-ray data were collected on a Bruker SMART-APEX CCD diffractometer (M. J. Zaworotko, Department of Chemistry, University of South Florida). Lattice parameters were determined from least squares analysis. Reflection data was integrated using the program SAINT. The structure was solved by direct methods and refined by full matrix least squares using the program SHELXTL (Sheldrick, G. M. SHELXTL, Release 5.03; Siemans Analytical X-ray Instruments Inc.: Madison, Wis.).

The co-crystals of the present invention can be characterized, e.g., by the TGA or DSC data or by any one, any two, any three, any four, any five, any six, any seven, any eight, any nine, any ten, or any single integer number of PXRD 2-theta angle peaks or Raman shift peaks listed herein or disclosed in a figure, or by single crystal x-ray diffraction data.

### Example 1

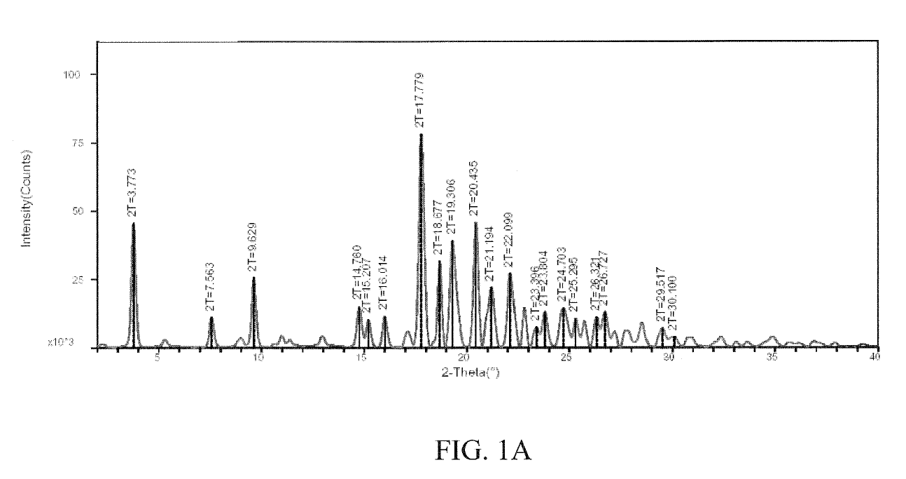

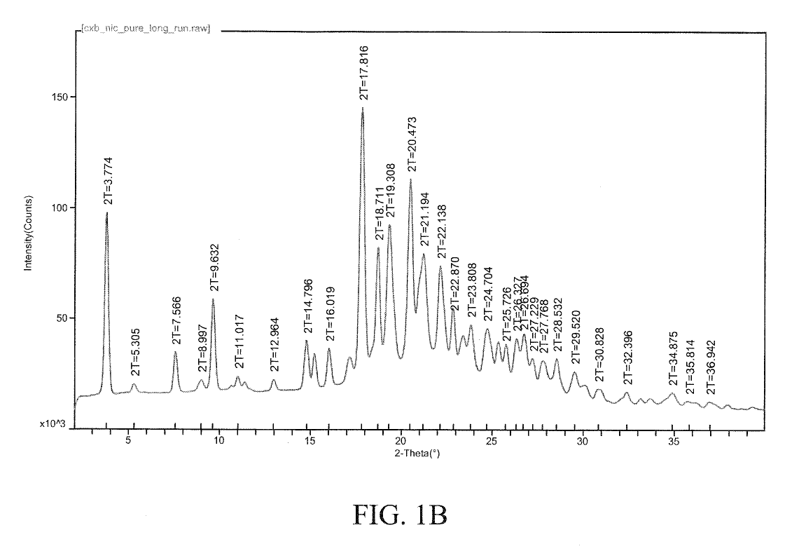

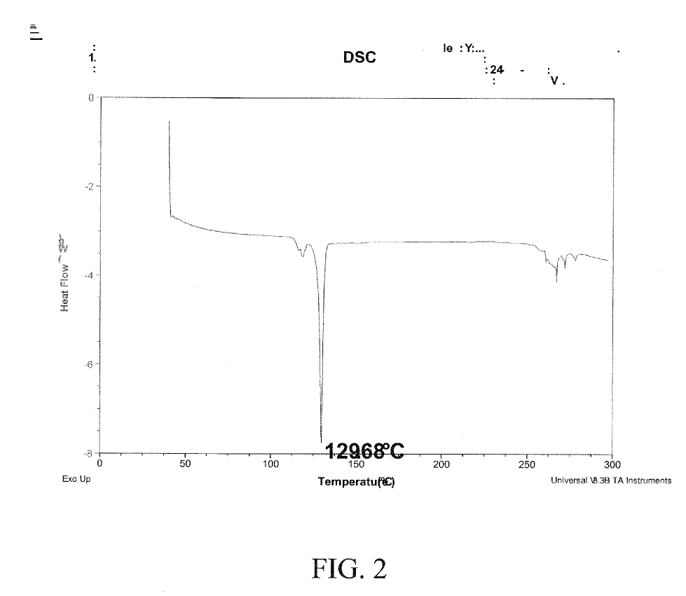

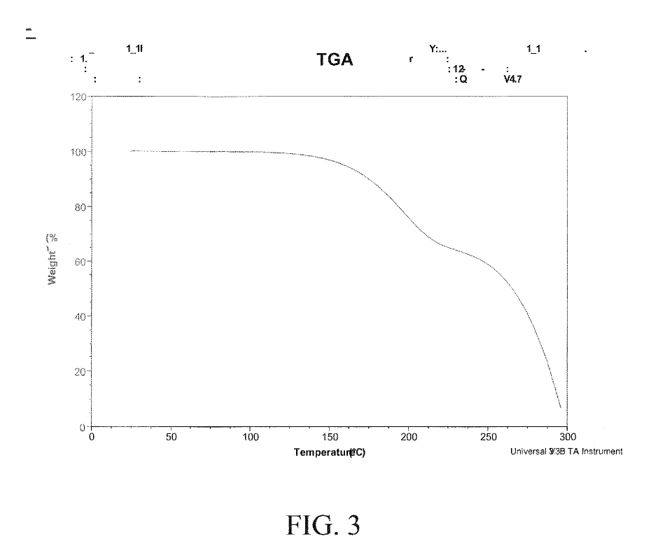

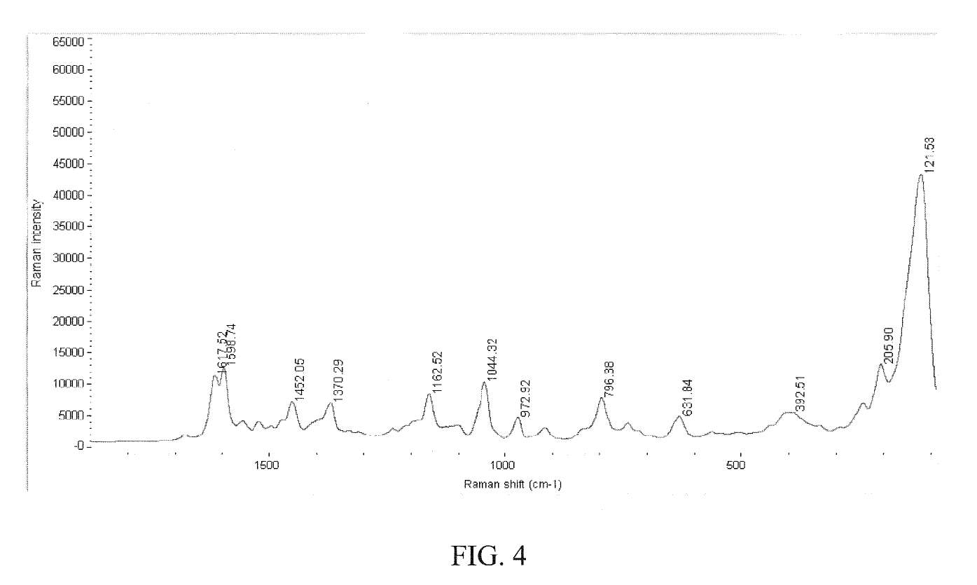

1:1 celecoxib:nicotinamide co-crystals were prepared. Celecoxib (100 mg, 0.26 mmol) and nicotinamide (32.0 mg, 0.26 mmol) were each dissolved in acetone (2 mL). The two solutions were mixed and the resulting mixture was allowed to evaporate slowly overnight. The precipitated solid was redissolved in acetone a second time and left to evaporate to dryness. The powder was collected and characterized. Detailed characterization of the celecoxib:nicotinamide co-crystal is listed in Table XXIV. FIG. 1A shows the PXRD diffractogram after subtraction of background noise. FIG. 1B shows the raw PXRD data. FIG. 2 shows a DSC thermogram of the celecoxib:nicotinamide co-crystal. FIG. 3 shows a TGA thermogram of the celecoxib:nicotinamide co-crystal. FIG. 4 shows a Raman spectrum of the celecoxib:nicotinamide co-crystal.

### Example 2

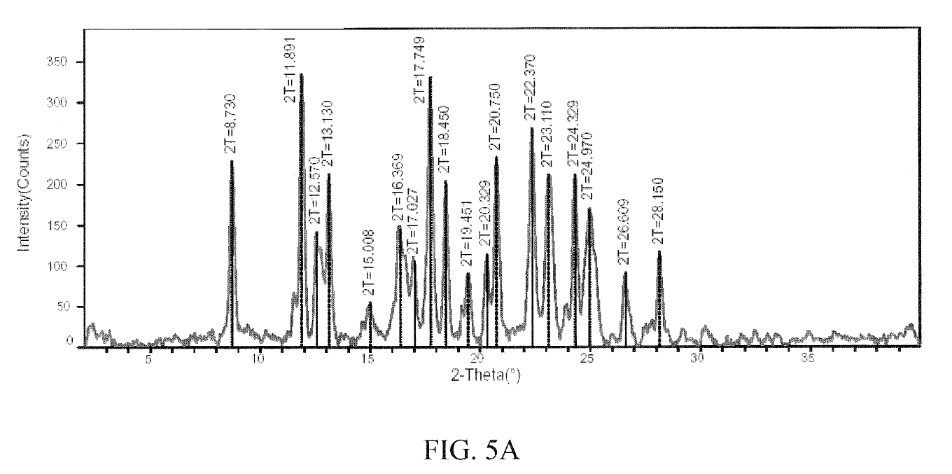

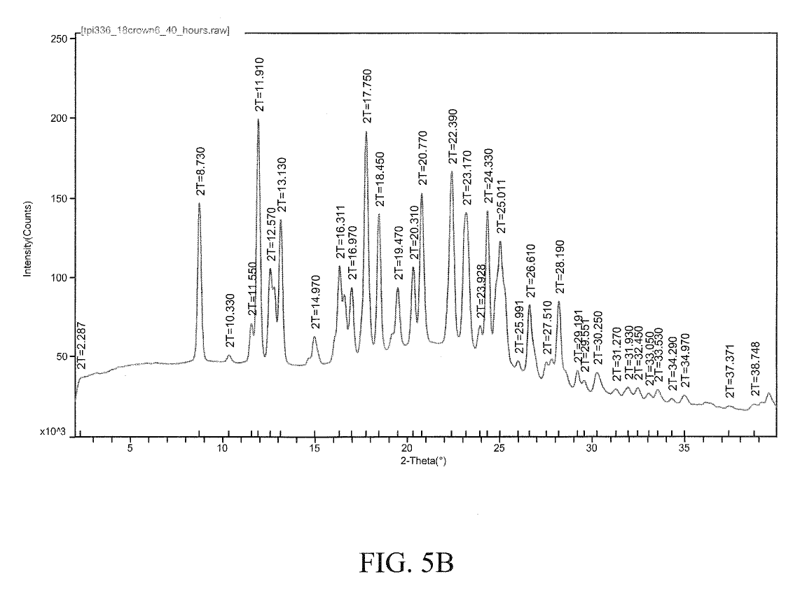

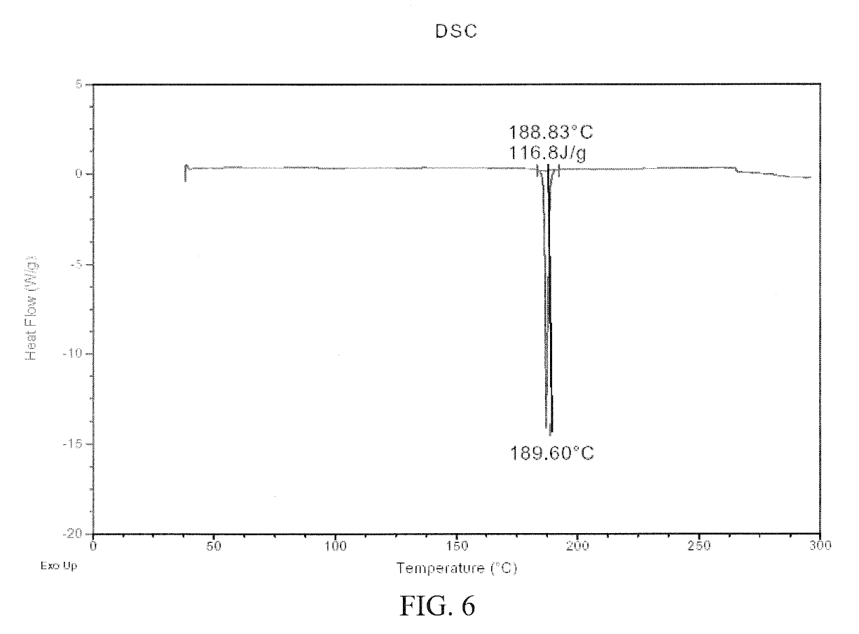

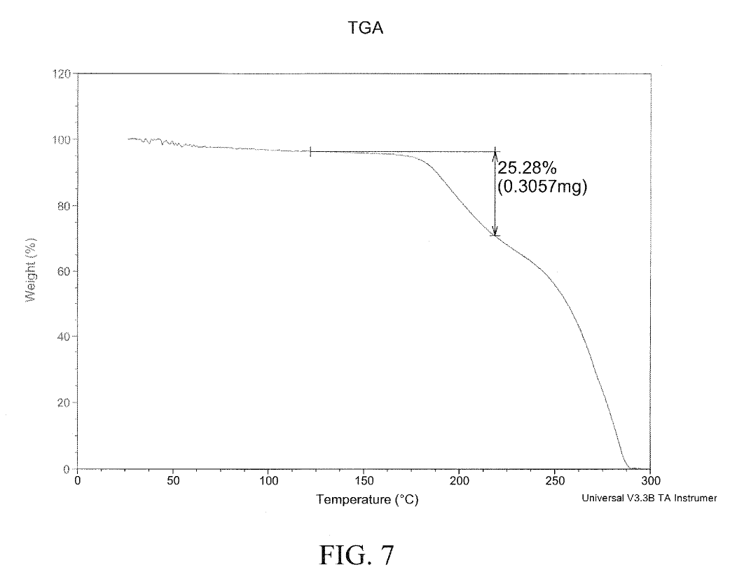

Co-crystals of celecoxib and 18-crown-6 were prepared. A solution of celecoxib (157.8 mg, 0.4138 mmol) in Et₂O (10.0 mL) was added to 18-crown-6 (118.1 mg, 0.447 mmol). The opaque solid dissolves immediately and a white solid subsequently began to crystallize very rapidly. The solid was collected via filtration and was washed with additional diethyl ether (5 mL). Detailed characterization of the celecoxib:18-crown-6 co-crystal is listed in Table XXIV. FIG. 5A shows the PXRD diffractogram after subtraction of background noise. FIG. 5B shows the raw PXRD data. FIG. 6 shows a DSC thermogram of the celecoxib:18-crown-6 co-crystal. FIG. 7 shows a TGA thermogram of the celecoxib:18-crown-6 co-crystal.

### Example 3

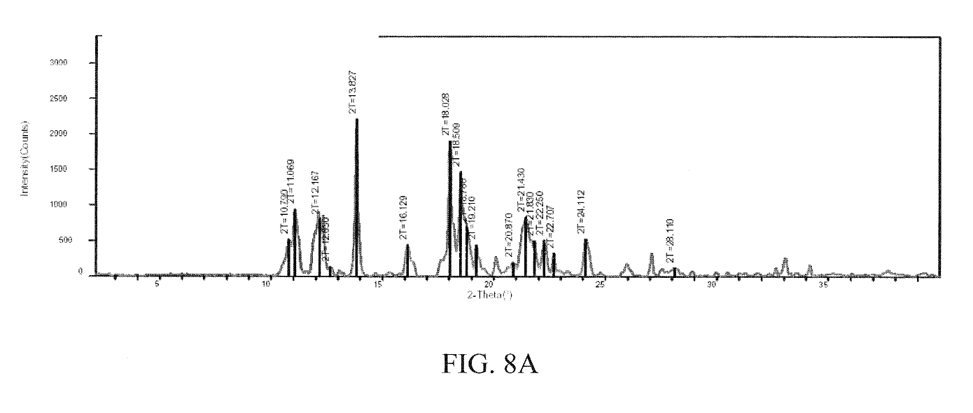

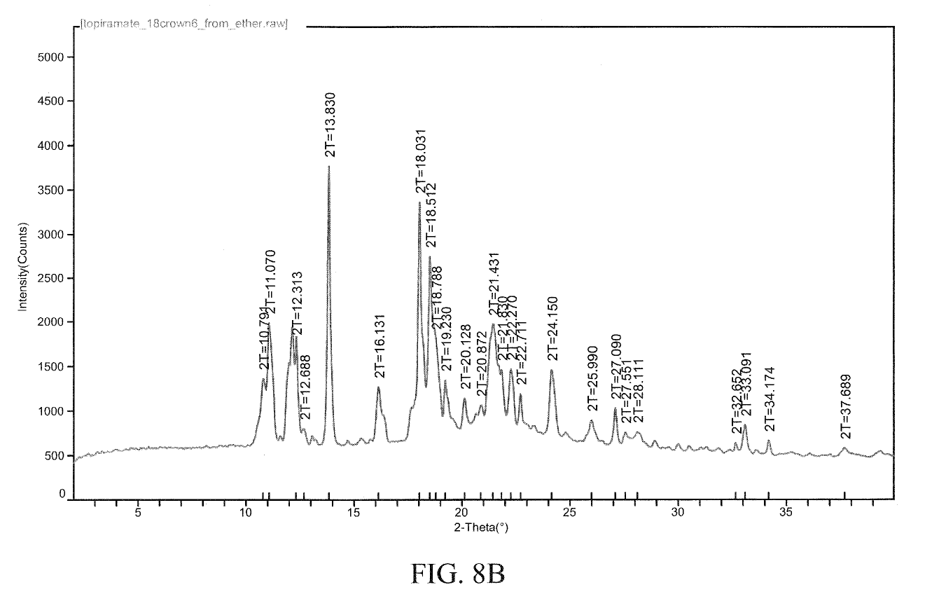

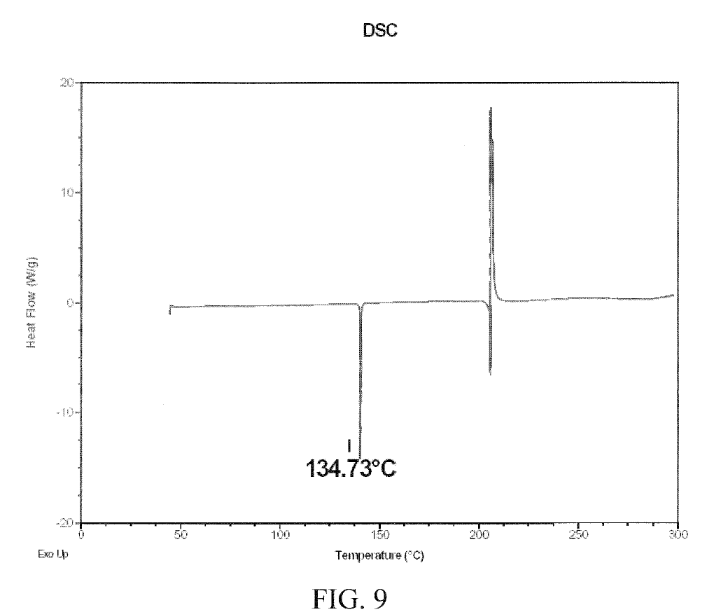

Co-crystals of topiramate and 18-crown-6 were prepared. To topiramate (100 mg, 0.29 mmol) dissolved in diethyl ether (5 mL) was added 18-crown-6 (78 mg, 0.29 mmol) in diethyl ether (5 mL). Upon addition of 18-crown-6, the solution became cloudy and was sonicated for 30 seconds. The solution was left standing for 1 hour and a colorless precipitate was observed. The precipitate was collected, washed with diethyl ether and dried to give a 1:1 co-crystal of topiramate:18-crown-6 as a colorless solid. Detailed characterization of the co-crystal is listed in Table XXIV. FIG. 8A shows the PXRD diffractogram after subtraction of background noise. FIG. 8B shows the raw PXRD data. FIG. 9 shows a DSC thermogram of the topiramate:18-crown-6 co-crystal.

### Example 4

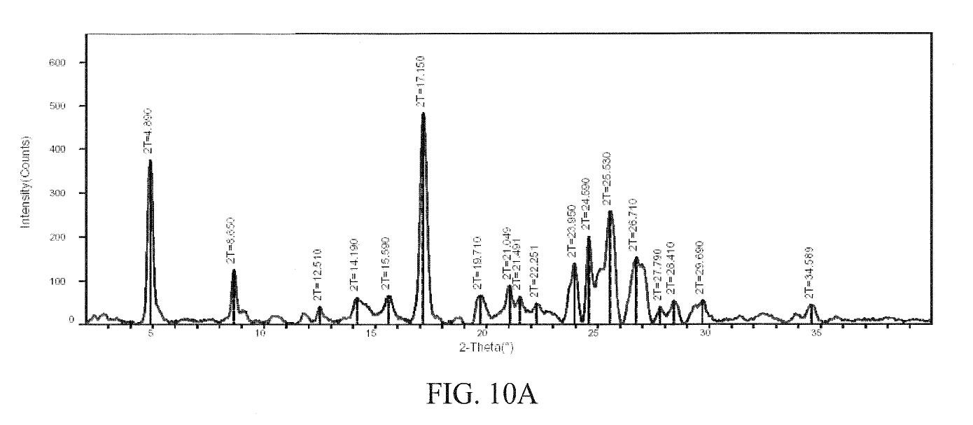

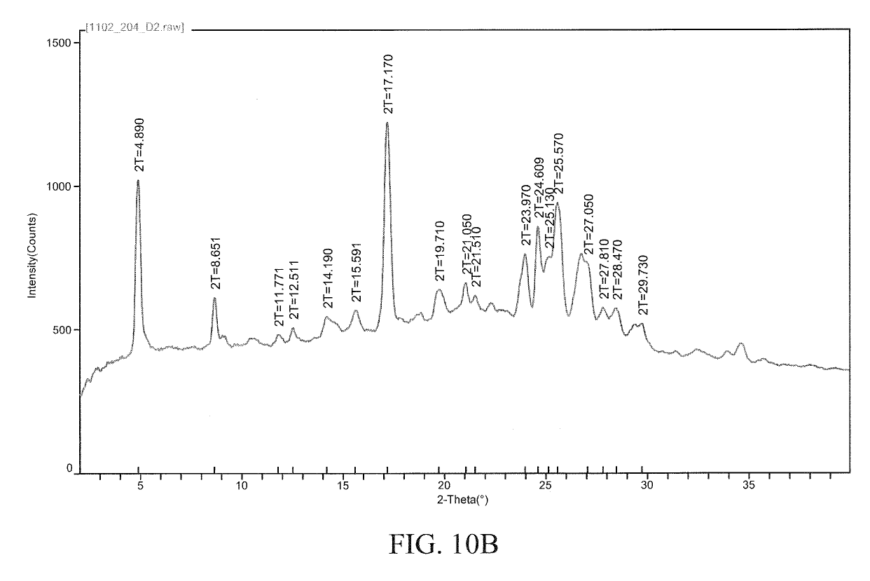

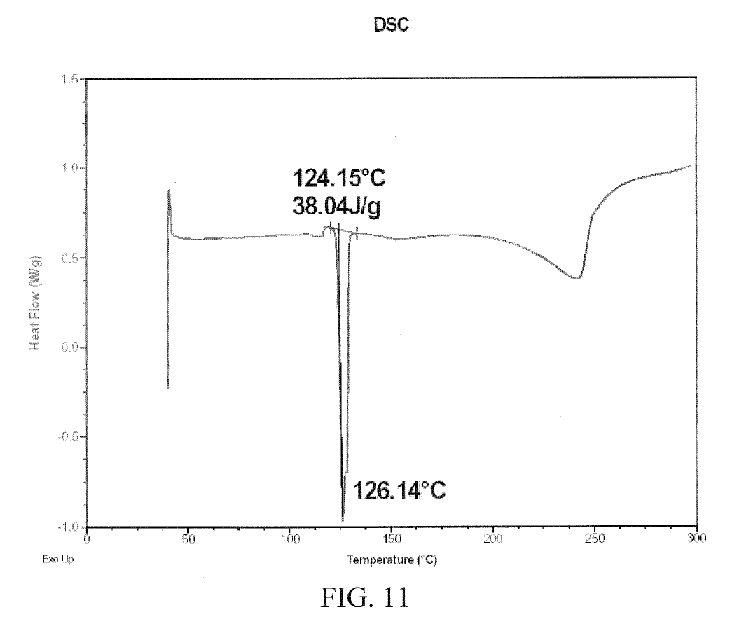

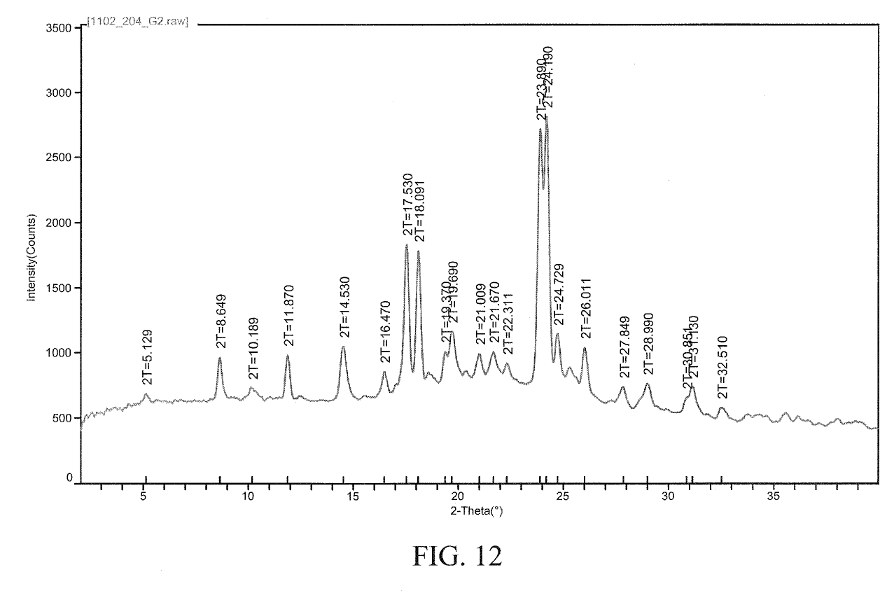

Co-crystals of olanzapine and nicotinamide (Forms I, II and III) were prepared. A 9-block experiment was designed with 12 solvents. (A block is a receiving plate, which can be, for example, an industry standard 24 well, 96 well, 384 well, or 1536 well format, or a custom format.) 864 crystallization experiments with 10 co-crystal formers and 3 concentrations were carried out using the CrystalMax™ platform. Form I was obtained from mixtures containing 1:1 and 1:2 molar ratios of olanzapine:nicotinamide in 1,2-dichloroethane. Form II was obtained from mixtures containing a 1:2 molar ratio of olanzapine and nicotinamide in isopropyl acetate. PXRD and DSC characterization of the olanzapine:nicotinamide co-crystals are listed in Table XXIV. FIG. 10A shows the PXRD diffractogram of form I after subtraction of background noise. FIG. 10B shows the raw PXRD data of form I. FIG. 11 shows a DSC thermogram of the olanzapine:nicotinamide form I co-crystal. FIG. 12 shows the PXRD diffractogram of olanzapine:nicotinamide form II after subtraction of background noise.

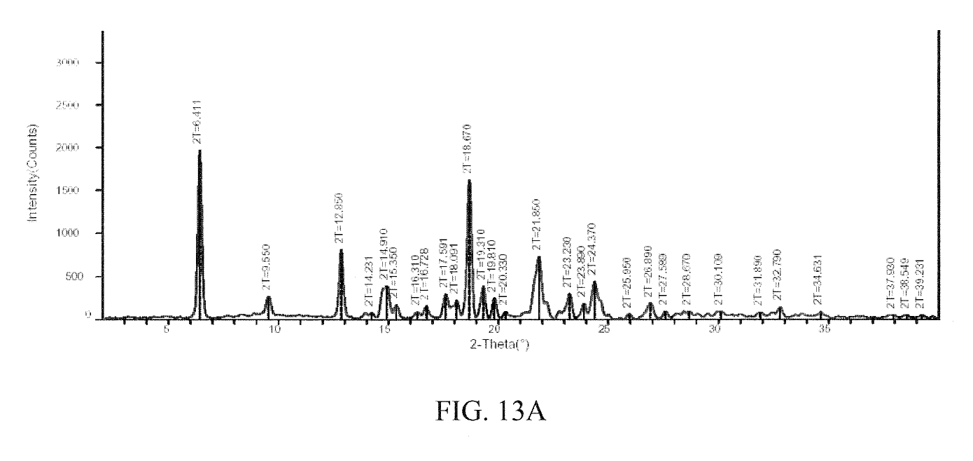

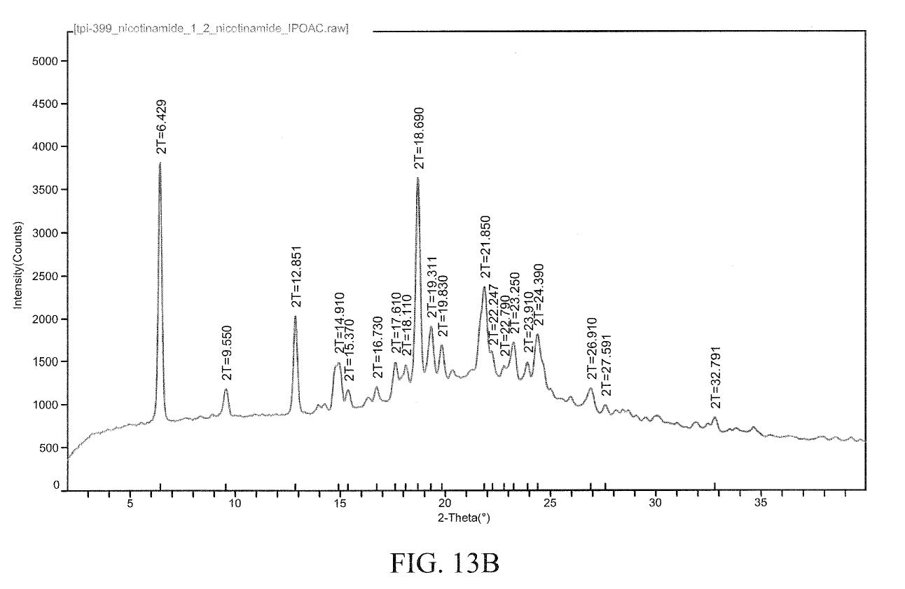

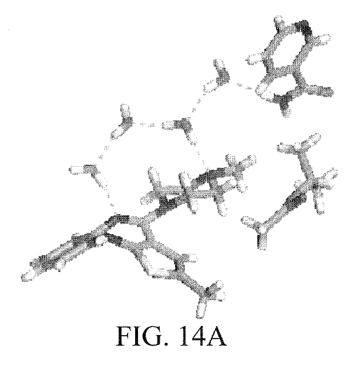

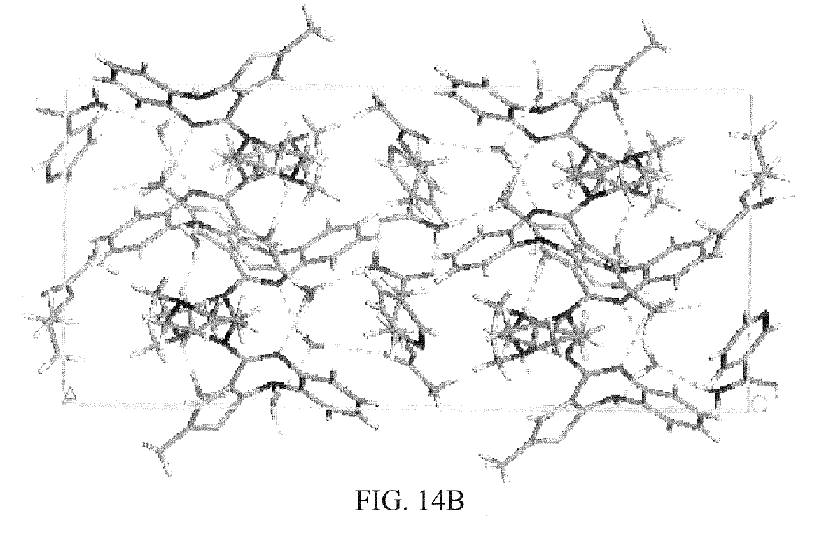

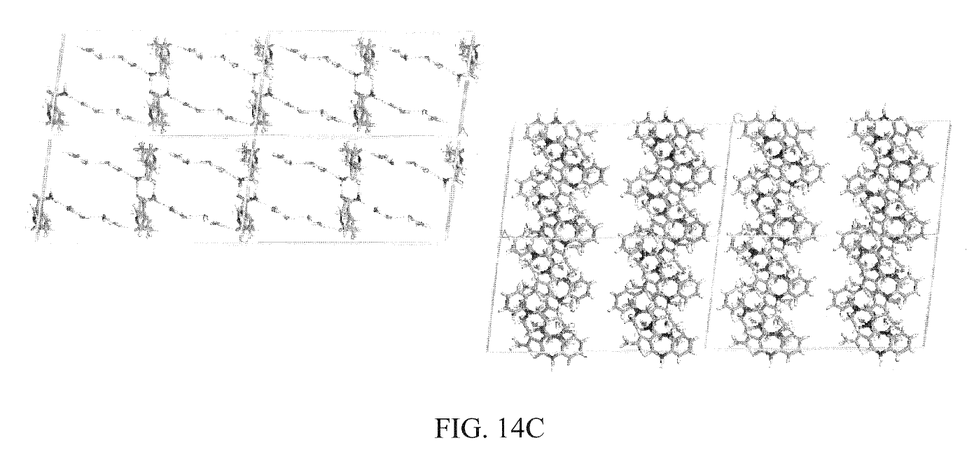

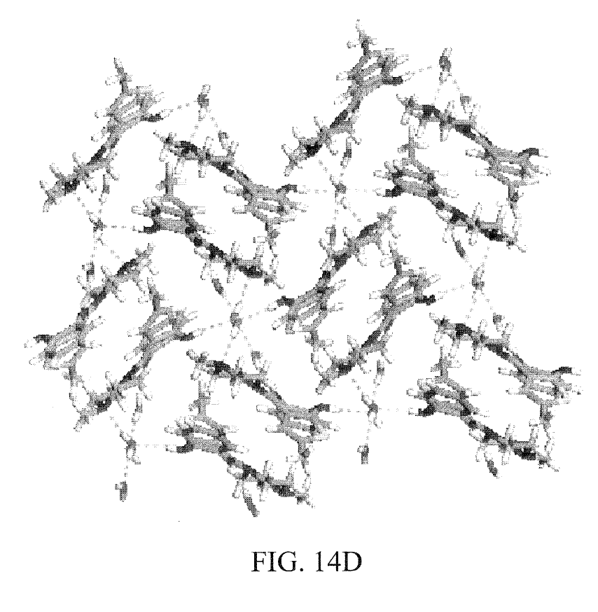

Co-crystals of olanzapine and nicotinamide (Form III) were prepared. Olanzapine (40 microliters of 25 mg/mL stock solution in tetrahydrofuran) and nicotinamide (37.6 microliters of 20 mg/mL stock solution in methanol) were added to a glass vial and dried under a flow of nitrogen. To the solid mixture was added isopropyl acetate (100 microliters) and the vial was sealed with an aluminum cap. The suspension was then heated at 70 degrees C. for two hours in order to dissolve all of the solid material. The solution was then cooled to 5 degrees C. and maintained at that temperature for 24 hours. After 24 hours the vial was uncapped and the mixture was concentrated to 50 microliters of total volume. The vial was then resealed with an aluminum cap and was maintained at 5 degrees C. for an additional 24 hours. Large, yellow plates were observed and were collected (Form III). The solid was characterized with single crystal x-ray diffraction and powder x-ray diffraction. PXRD characterization of the co-crystal is listed in Table XXIV. FIG. 13A shows the PXRD diffractogram of form III after subtraction of background noise. FIG. 13B shows the raw PXRD data of form III. FIGS. 14A-D show packing diagrams of the olanzapine:nicotinamide form III co-crystal.

Single crystal x-ray analysis reveals that the olanzapine:nicotinamide (Form III) co-crystal is made up of a ternary system containing olanzapine, nicotinamide, water and isopropyl acetate in the unit cell. The co-crystal crystallizes in the monoclinic space group P2₁/c and contains two olanzapine molecules, one nicotinamide molecule, 4 water molecules and one isopropyl acetate molecule in the asymmetric unit. The packing diagram is made up of a two-dimensional hydrogen-bonded network with the water molecules connecting the olanzapine and nicotinamide moieties. The packing diagram is also comprised of alternating olanzapine and nicotinamide layers connected through hydrogen bonding via the water and isopropyl acetate molecules, as shown in FIG. 14B. The olanzapine layer propagates along the b axis at c/4 and 3c/4. The nicotinamide layer propagates along the b axis at c/2. The top of FIG. 14C illustrates the nicotinamide superstructure. The nicotinamide molecules form dimers which hydrogen bond to chains of 4 water molecules. The water chains terminate with isopropyl acetate molecules on each side.

Crystal data: C₄₅H₆₄N₁₀O₇S₂, M=921.18, monoclinic P21/c; a=14.0961(12) Å, b=12.5984(10) Å, c=27.219(2) Å, α=90°, β=97.396(2)°, γ=90°, T=100(2) K, Z=4, D_{c}=1.276 Mg/m³,U=4793.6(7) Å³, λ=0.71073 Å; 24952 reflections measured, 8457 unique (Rᵢₙₜ=0.0882). Final residuals were R₁=0.0676, wR₂=0.1461 for I>2σ(I), and R₁=0.1187, wR₂=0.1687 for all 8457 data.

### Example 5

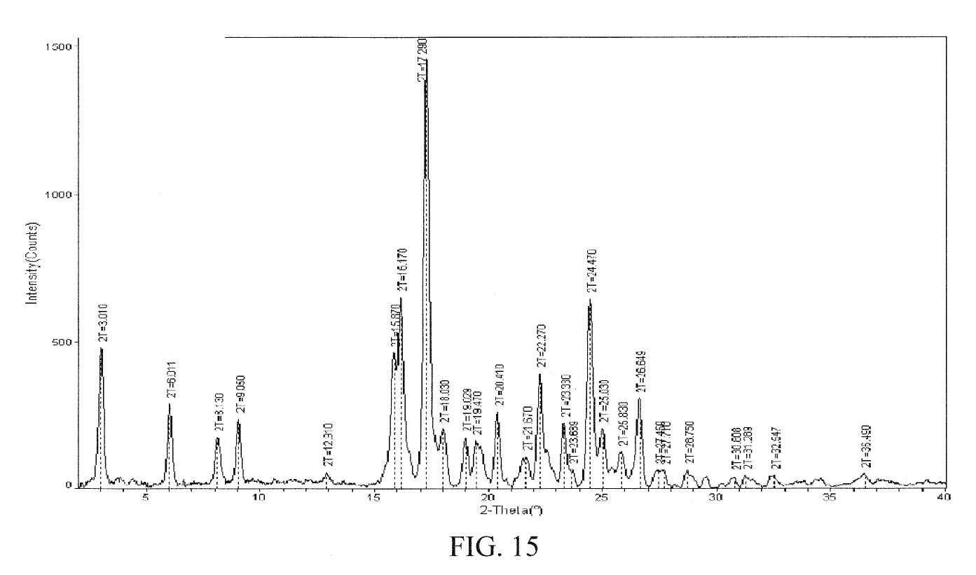

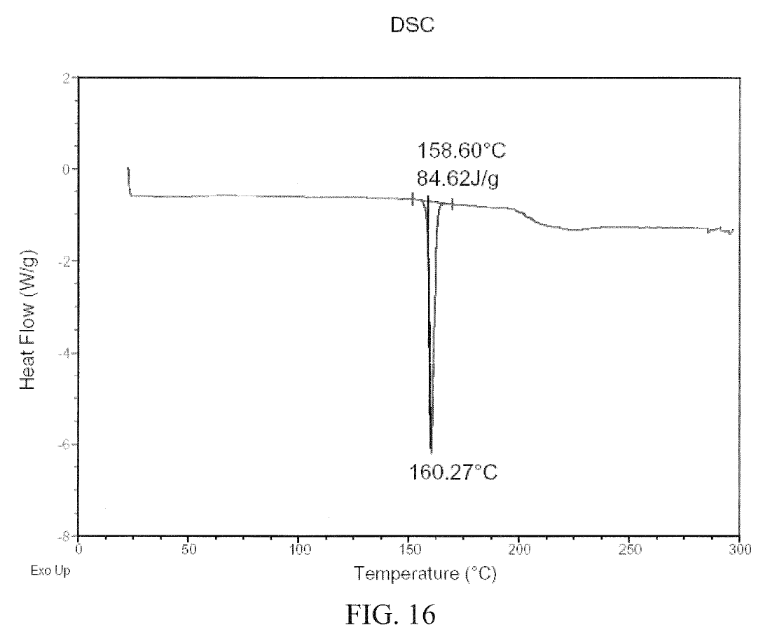

A co-crystal of cis-itraconazole and succinic acid was prepared. To a solution of succinic acid (16.8 mg, 0.142 mmol) in tetrahydrofuran (THF) (0.50 mL) was added cis-itraconazole (100 mg, 0.142 mmol). A clear solution formed with heating (60 degrees C.) and stirring. Upon cooling to room temperature (25 degrees C.), crystals began to form. The solid was collected by filtration and washed with cold THF (2 mL). The white solid was air-dried and placed in a glass vial. The crystalline substance was found to be a succinic acid co-crystal of cis-itraconazole. The solid was characterized by PXRD and DSC. FIG. 15 shows the PXRD diffractogram after subtraction of background noise. FIG. 16 shows a DSC thermogram of the co-crystal.

### Example 6

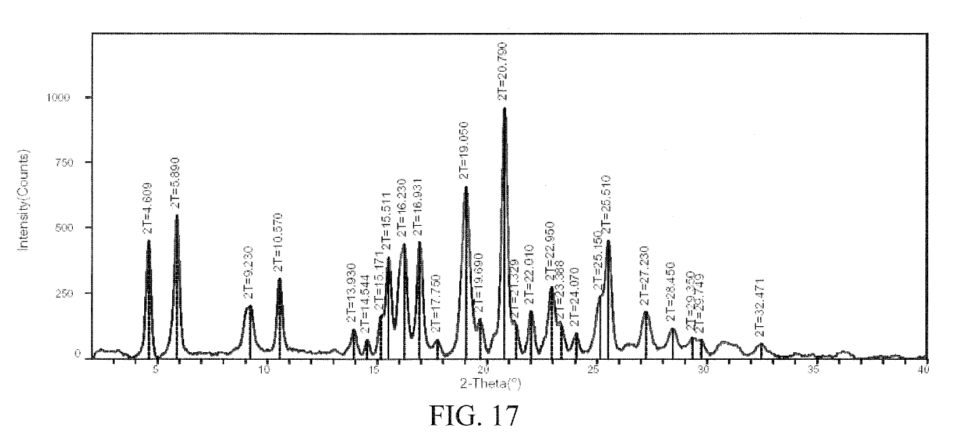

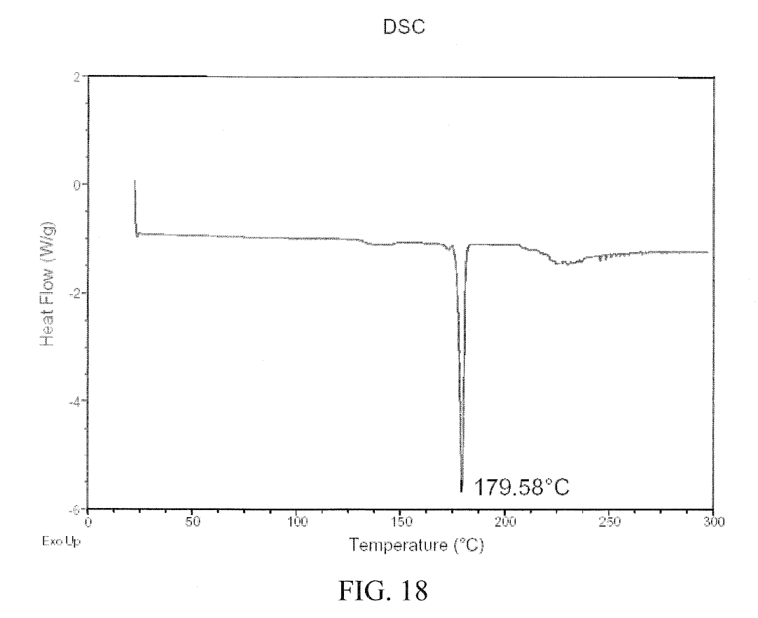

A co-crystal of cis-itraconazole and fumaric acid was prepared. To a blend of fumaric acid (8.40 mg, 0.072 mmol) and cis-itraconazole (51.8 mg, 0.073 mmol) was added tetrahydrofuran (THF) (1.0 mL). A clear solution formed with heating (60 degrees C.) and stirring. Upon cooling to room temperature (25 degrees C.), no crystals formed. To the clear solution was added t-butyl methyl ether (1.0 mL). A white solid formed immediately and was collected by filtration and washed with cold t-butyl methyl ether (2 mL). The white solid was air-dried and placed in a glass vial. The crystalline substance was found to be a fumaric acid co-crystal of cis-itraconazole. The solid was characterized by PXRD and DSC. FIG. 17 shows the PXRD diffractogram after subtraction of background noise. FIG. 18 shows a DSC thermogram of the co-crystal.

### Example 7

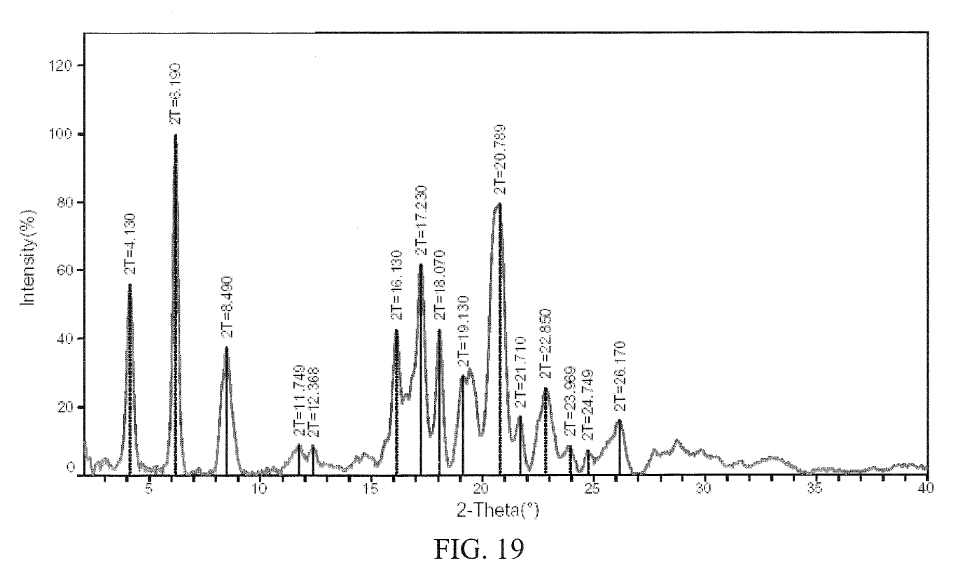

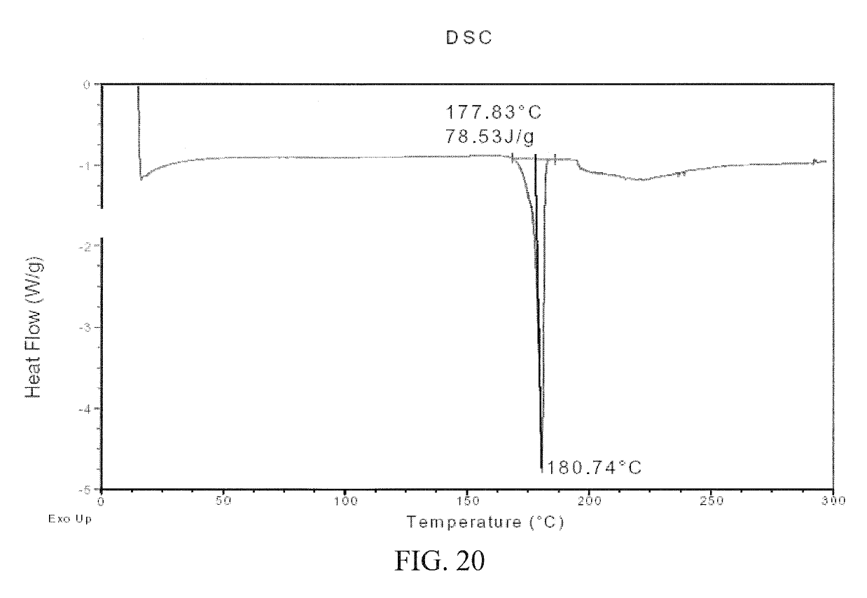

A co-crystal of cis-itraconazole and L-tartaric acid was prepared. To a solution of L-tartaric acid (21.3 mg, 0.142 mmol) in tetrahydrofuran (THF) (0.50 mL) was added cis-itraconazole (100 mg, 0.142 mmol). A clear solution formed with heating (60 degrees C.) and stirring. Upon cooling to room temperature (25 degrees C.), crystals began to form. The solid was collected by filtration and washed with cold THF (2 mL). The white solid was air-dried and placed in a glass vial. The crystalline substance was found to be an L-tartaric acid co-crystal of cis-itraconazole. The solid was characterized by PXRD and DSC. FIG. 19 shows the PXRD diffractogram after subtraction of background noise. FIG. 20 shows a DSC thermogram of the co-crystal.

### Example 8

A co-crystal of cis-itraconazole and L-malic acid was prepared. To a solution of L-malic acid (19.1 mg, 0.143 mmol) in tetrahydrofuran (THF) (0.50 mL) was added cis-itraconazole (100 mg, 0.142 mmol). A clear solution formed with heating (60 degrees C.) and stirring. Upon cooling to room temperature (25 degrees C.), crystals began to form. The solid was collected by filtration and washed with cold THF (2 mL). The white solid was air-dried and placed in a glass vial. The crystalline substance was found to be an L-malic acid co-crystal of cis-itraconazole. The solid was characterized by PXRD and DSC.

### Example 9

A co-crystal of cis-itraconazole hydrochloride and DL-tartaric acid was prepared. To a suspension of cis-itraconazole freebase (20.1 g, 0.0285 mol) in absolute ethanol (100 mL) was added a solution of hydrochloric acid (1.56 g, 0.0428 mol) and DL-tartaric acid (2.99 g, 0.0171 mol) in absolute ethanol (100 mL). A clear solution formed with stirring and heating to reflux. The hot solution was gravity filtered and allowed to cool to room temperature (25 degrees C.). Upon cooling white crystals formed. The solid was collected by filtration and washed with cold absolute ethanol (15 mL). The white solid was dried in a vacuum oven overnight at 80 degrees C. The crystalline substance was found to be a DL-tartaric acid co-crystal of cis-itraconazole hydrochloride. The solid was characterized by PXRD and DSC.

### Example 10

Co-crystals of modafinil and malonic acid were prepared. Using a 250 mg/ml modafinil-acetic acid solution, malonic acid was dissolved on a hotplate (about 67 degrees C.) at a 1:2 modafinil to malonic acid ratio. The mixture was dried under flowing nitrogen overnight. A powdery white solid was produced. After further drying for 1 day, acetic acid was removed (as determined by TGA) and the crystal structure of the modafinil:malonic acid (Form I) co-crystal, as determined by PXRD, remained the same. The modafinil:malonic acid (Form I) co-crystal was also prepared by grinding the API and co-crystal former together. 2.50 g of modafinil was mixed with 1.01 g of malonic acid in a large mortar and pestle (malonic acid added in increments over 7 days with about a 1:1.05 ratio made on the first day and increments added over the next seven days which resulted in a 1:2 modafinil:malonic acid ratio). The mixture was ground for 45 minutes initially and 20 minutes each time more malonic acid was added. On the seventh day the mixture of co-crystal and starting components was heated in a sealed 20 mL vial at 80 degrees C. for about 35 minutes to facilitate completion of the co-crystal formation. PXRD analysis of the resultant material was completed and the diffractogram is shown after subtraction of background noise. The Raman spectrum of the modafinil:malonic acid Form I co-crystal comprises peaks, in order of decreasing intensity, of 1004, 222, 633, 265, 1032, 1183, 814, 1601, 490, 718, 767, 361, 917, 1104, 889, 412, 1225, 1251, 1398, 1442, 1731, 1298, 3065, and 2949 cm⁻¹. Single crystal data of the modafinil:malonic acid Form I co-crystal were acquired and are reported below.

Crystal data: C₁₈H₁₉NO₆S, M=377.40, monoclinic C2/c; a=18.728(8) angstroms, b=5.480(2) angstroms, c=33.894(13) angstroms, alpha=90 degrees, beta=91.864(9) degrees, gamma=90 degrees, T=100(2) K, Z=8, D_{c}=1.442 Mg/m³, U=3477(2) cubic angstroms, λ=0.71073 angstroms, 6475 reflections measured, 3307 unique (Rᵢₙₜ=0.1567). Final residuals were R₁=0.1598, wR₂=0.3301 for I>2sigma(I), and R₁=0.2544, wR₂=0.3740 for all 3307 data.

A polymorph of the modafinil:malonic acid Form I co-crystal was prepared in a vial. 11.4 mg of modafinil and 8.9 mg of malonic acid were dissolved in 2 mL of acetone. The solids dissolved at room temperature, and the vial was left open to evaporate the solvent in air. Large parallelogram shaped crystals formed on the walls and bottom of the vial. The PXRD diffractogram of the large crystals showed modafinil:malonic acid co-crystals Form II, a polymorphic form of modafinil:malonic acid Form I.

### Example 11

Co-crystals of modafinil and glycolic acid were prepared. Modafinil (1 mg, 0.0037 mmol) and glycolic acid (0.30 mg, 0.0037 mmol) were dissolved in acetone (400 microliters). The solution was allowed to evaporate to dryness and the resulting solid was characterized using PXRD. PXRD data for the modafinil:glycolic acid co-crystal is listed in Table XXIV.

### Example 12

Co-crystals of modafinil and maleic acid were prepared. Using a 250 mg/ml modafinil-acetic acid solution, maleic acid was dissolved on a hotplate (about 67 degrees C.) at a 2:1 modafinil to maleic ratio. The mixture was dried under flowing nitrogen overnight. A clear amorphous material remained. Solids began to grow after 2 days stored in a sealed vial at room temperature. The solid was collected and characterized as the modafinil:maleic acid co-crystal using PXRD.

### Example 13

Co-crystals of 5-fluorouracil and urea were prepared. To 5-fluorouracil (1 g, 7.69 mmol) and urea (0.46 g, 7.69 mmol) was added methanol (100 mL). The solution was heated at 65 degrees C. and sonicated until all the material dissolved. The solution was then cooled to 5 degrees C. and maintained at that temperature overnight. After about 3 days a white precipitate was observed and collected. The solid was characterized by DSC, PXRD, Raman spectroscopy, and TGA as the 5-fluorouracil:urea co-crystal. Characterization data are listed in Table XXIV. Single crystal data of the 5-fluorouracil:urea co-crystal were acquired and are reported below.

Crystal data: C₅H₇FN₄O₃, M=190.15, monoclinic C2/C, a=9.461(3) angstroms, b=10.487(3) angstroms, c=15.808(4) angstroms, alpha=90 degrees, beta=99.891(5), gamma=90 degrees, T=100(2) K, Z=8, D_{c}=1.635 Mg/m³, U=1545.2(7) cubic angstroms, λ=0.71073 angstroms, 3419 reflections measured, 1633 unique (Rᵢₙₜ=0.0330). Final residuals were R₁=0.0667, wR₂=0.1505 for I>2sigma(I), and R₁=0.0872, wR₂=0.1598 for all 1633 data.

### Example 14

Co-crystals of hydrochlorothiazide and nicotinic acid were prepared. Hydrochlorothiazide (12.2 mg, 0.041 mmol) and nicotinic acid (5 mg, 0.041 mmol) were dissolved in methanol (1 mL). The solution was then cooled to 5 degrees C. and maintained at that temperature for 12 hours. A white solid precipitated and was collected and characterized as the hydrochlorothiazide:nicotinic acid co-crystal using PXRD.

### Example 15

Co-crystals of hydrochlorothiazide and 18-crown-6 were prepared. Hydrochlorothiazide (100 mg, 0.33 mmol) was dissolved in diethyl ether (15 mL) and was added to a solution of 18-crown-6 (87.2 mg, 0.33 mmol) in diethyl ether (15 mL). A white precipitate immediately began to form and was collected and characterized as the hydrochlorothiazide:18-crown-6 co-crystal using PXRD.

### Example 16

Co-crystals of hydrochlorothiazide and piperazine were prepared. Hydrochlorothiazide (17.3 mg, 0.058 mmol) and piperazine (5 mg, 0.058 mmol) were dissolved in a 1:1 mixture of ethyl acetate and acetonitrile (1 mL), The solution was then cooled to 5 degrees C. and maintained at that temperature for 12 hours. A white solid precipitated and was collected and characterized as the hydrochlorothiazide:piperazine co-crystal using PXRD.

### Example 17

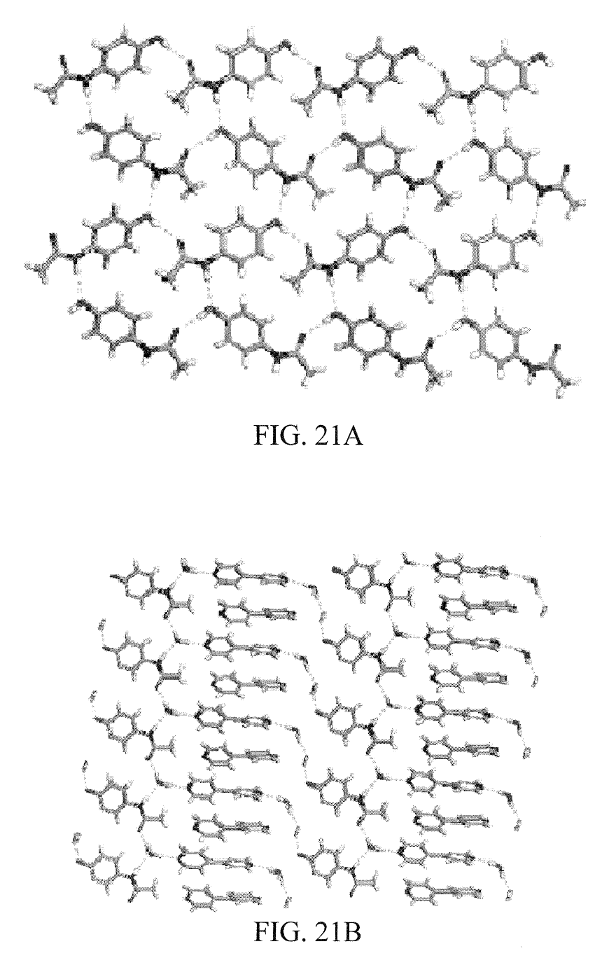

Acetaminophen:4,4′-bipyridine:water (1:1:1 Stoichiometry) 50 mg (0.3307 mmol) acetaminophen and 52 mg (0.3329 mmol) 4,4′-bipyridine were dissolved in hot water and allowed to stand. Slow evaporation yielded colorless needles of a 1:1:1 acetaminophen:4,4′-bipyridine:water co-crystal, as shown in FIGS. 21A-B.

Crystal data: (Bruker SMART-APEX CCD Diffractometer). triclinic, space group PĪ; a=7.0534(8), b=9.5955(12), c=19.3649(2) Å, α=86.326(2), β=80.291(2), γ=88.880(2)°, U=1308.1(3) Å³, T=200(2) K, Z=2, μ(Mo—Kα)=0.090 mm⁻¹, D_{c}=1.294 Mg/m³, λ=0.71073 Å, F(000)=537, 2θₘₐₓ=25.02°; 6289 reflections measured, 4481 unique (Rᵢₙₜ=0.0261). Final residuals for 344 parameters were R₁=0.0751, wR₂=0.2082 for I>2σ(I), and R₁=0.1119, wR₂=0.2377 for all 4481 data.

Crystal packing: The co-crystals contain bilayered sheets in which water molecules act as a hydrogen bonded bridge between the network bipyridine moieties and the acetaminophen. Bipyridine guests are sustained by π-π stacking interactions between two network bipyridines. The layers stack via π-π interactions between the phenyl groups of the acetaminophen moieties.

Differential Scanning Calorimetry: (TA Instruments 2920 DSC), 57.77 degrees C. (endotherm); m.p.=58-60 degrees C. (MEL-TEMP); (acetaminophen m.p.=169 degrees C., 4,4′-bipyridine m.p.=111-114 degrees C.).

### Example 18

### Phenyloin:Pyridone (1:1 Stoichiometry)

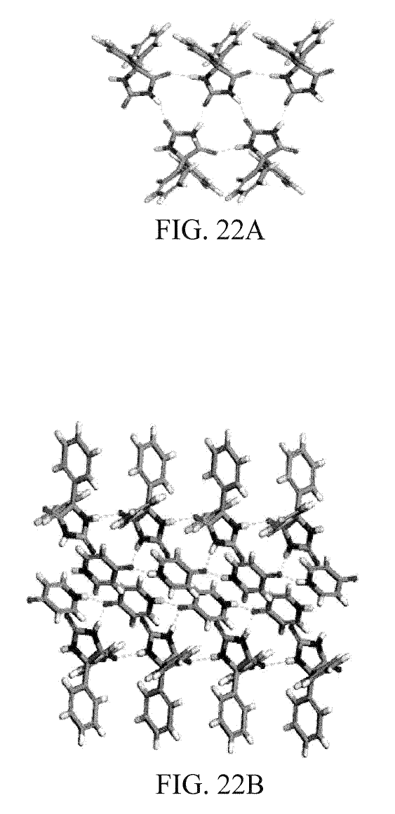

28 mg (0.1109 mmol) phenyloin and 11 mg (0.1156 mmol) 4-hydroxypyridone were dissolved in 2 mL acetone and 1 mL ethanol with heating and stirring. Slow evaporation yielded colorless needles of a 1:1 phenytoin:pyridone co-crystal, as shown in FIGS. 22A-B.

Crystal data: (Bruker SMART-APEX CCD Diffractometer), C₂₀H₁₇N₃O₃, M=347.37, monoclinic P2₁/c; a=16.6583(19), b=8.8478(10), c=11.9546(14) Å, β=96.618(2)°, U=1750.2(3) Å³, T=200(2) K, Z=4, μ(Mo—Kα)=0.091 mm⁻¹, D_{c}=1.318 Mg/m³, λ=0.71073 Å, F(000)=728, 2θₘₐₓ=56.60°; 10605 reflections measured, 4154 unique (Rᵢₙₜ=0.0313). Final residuals for 247 parameters were R₁=0.0560, wR₂=0.1356 for I>2σ(I), and R₁=0.0816, wR₂=0.1559 for all 4154 data.

Crystal packing: The co-crystal is sustained by hydrogen bonding of adjacent phentoin molecules between the carbonyl and the amine closest to the tetrahedral carbon, and by hydrogen bonding between pyridone carbonyl functionalities and the amine not involved in phenyloin-phenyloin interactions. The pyridone carbonyl also hydrogen bonds with adjacent pyridone molecules forming a one-dimensional network.

Infrared Spectroscopy: (Nicolet Avatar 320 FTIR), characteristic peaks for the co-crystal were identified as: 2° amine found at 3311 cm⁻¹, carbonyl (ketone) found at 1711 cm⁻¹, olephin peak found at 1390 cm⁻¹.

Differential Scanning Calorimetry: (TA Instruments 2920 DSC), 233.39 degrees C. (endotherm) and 271.33 degrees C. (endotherm); m.p.=231-233 degrees C. (MEL-TEMP); (phenyloin m.p.=295 degrees C., pyridone m.p.=148 degrees C.).

Thermogravimetric Analysis: (TA Instruments 2950 Hi-Resolution TGA), a 29.09% weight loss starting at 192.80 degrees C., 48.72% weight loss starting at 238.27 degrees C., and 18.38% loss starting at 260.17 degrees C. followed by complete decomposition. Powder x-ray diffraction: (Rigaku. Miniflex Diffractometer using Cu Kα (λ=1.540562), 30 kV, 15 mA). The powder data were collected over an angular range of 3° to 40° 2θ in continuous scan mode using a step size of 0.02° 2θ and a scan speed of 2.0°/minute. PXRD: Showed analogous peaks to the simulated PXRD derived from the single crystal data. experimental (calculated): 5.2 (5.3); 11.1 (11.3); 15.1 (15.2); 16.2 (16.4); 16.7 (17.0); 17.8 (17.9); 19.4 (19.4); 19.8 (19.7); 20.3 (20.1); 21.2 (21.4); 23.3 (23.7); 26.1 (26.4); 26.4 (26.6); 27.3 (27.6); 29.5 (29.9).

### Example 19

### Aspirin (acetylsalicylic acid):4,4′-bipyridine (2:1 Stoichiometry)

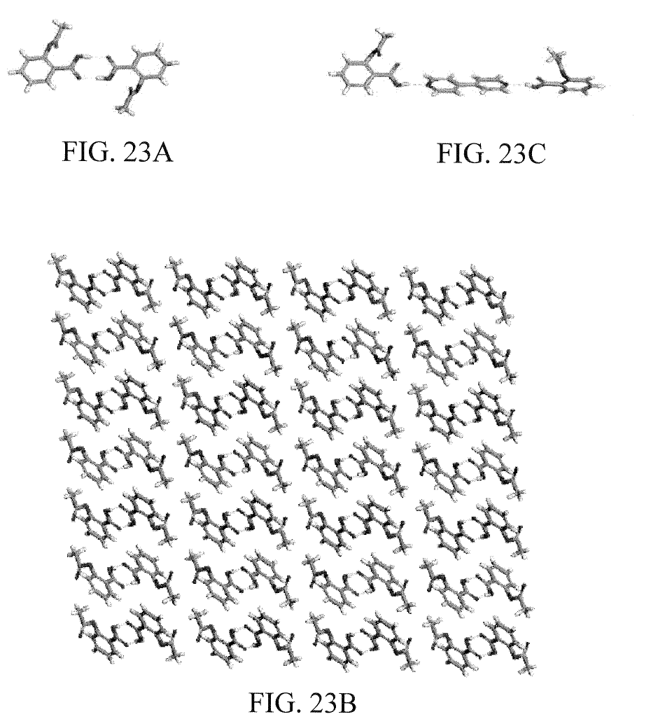

50 mg (0.2775 mmol) aspirin and 22 mg (0.1388 mmol) 4,4′-bipyridine were dissolved in 4 mL hexane. 8 mL ether was added to the solution and allowed to stand for one hour, yielding colorless needles of a 2:1 aspirin:4,4′-bipyridine co-crystal, as shown in FIGS. 23A-D. Alternatively, aspirin:4,4′-bipyridine (2:1 stoichiometry) can be made by grinding the solid ingredients in a pestle and mortar.

Crystal data: (Bruker SMART-APEX CCD Diffractometer), C₂₈H₂₄N₂O₈, M=516.49, orthorhombic Pbcn; a=28.831(3), b=11.3861(12), c=8.4144(9) Å, U=2762.2(5) Å³, T=173(2) K, Z=4, μ(Mo—Kα)=0.092 mm⁻¹, D_{c}=1.242 Mg/m³, λ=0.71073 Å, F(000)=1080, 2θₘₐₓ=25.02°; 12431 reflections measured, 2433 unique (Rᵢₙₜ=0.0419). Final residuals for 202 parameters were R₁=0.0419, wR₂=0.1358 for I>2σ(I), and R₁=0.0541, wR₂=0.1482 for all 2433 data.

Crystal packing: The co-crystal contains the carboxylic acid-pyridine heterodimer that crystallizes in the Pbcn space group. The structure is an inclusion compound containing disordered solvent in the channels. In addition to the dominant hydrogen bonding interaction of the heterodimer, π-π (stacking of the bipyridine and phenyl groups of the aspirin and hydrophobic interactions contribute to the overall packing interactions.

Infrared Spectroscopy: (Nicolet Avatar 320 FTIR), characteristic (—COOH) peak at 1679 cm⁻¹ was shifted up and less intense at 1694 cm⁻¹, where as the lactone peak is shifted down slightly from 1750 cm⁻¹ to 1744 cm⁻¹.

Differential Scanning Calorimetry: (TA Instruments 2920 DSC), 95.14 degrees C. (endotherm); m.p.=91-96 degrees C. (MEL-TEMP); (aspirin m.p.=1345 degrees C., 4,4′-bipyridine m.p.=111-114 degrees C.).

Thermogravimetric Analysis: (TA Instruments 2950 Hi-Resolution TGA), weight loss of 9% starting at 22.62 degrees C., 49.06% weight loss starting at 102.97 degrees C. followed by complete decomposition starting at 209.37 degrees C.

### Example 20

### Ibuprofen:4,4′-Bipyridine (2:1 Stoichiometry)

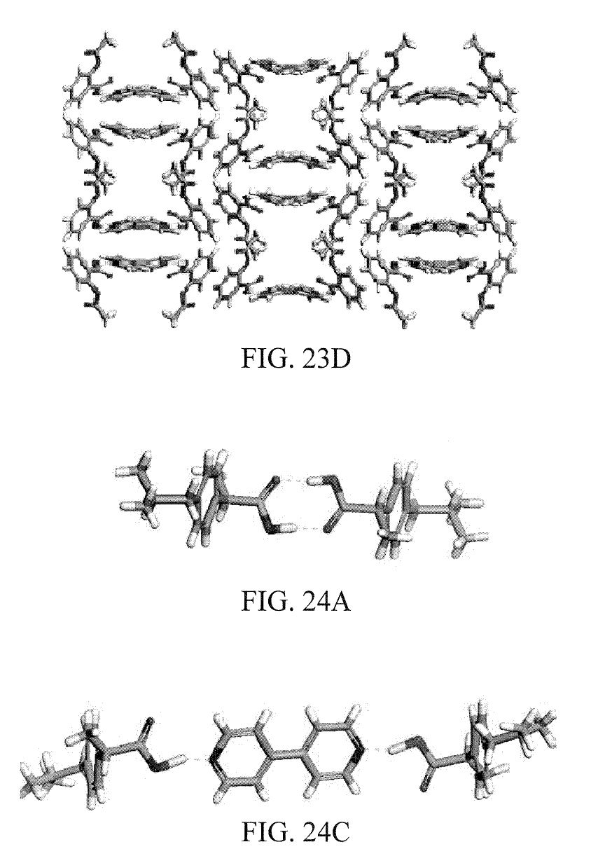

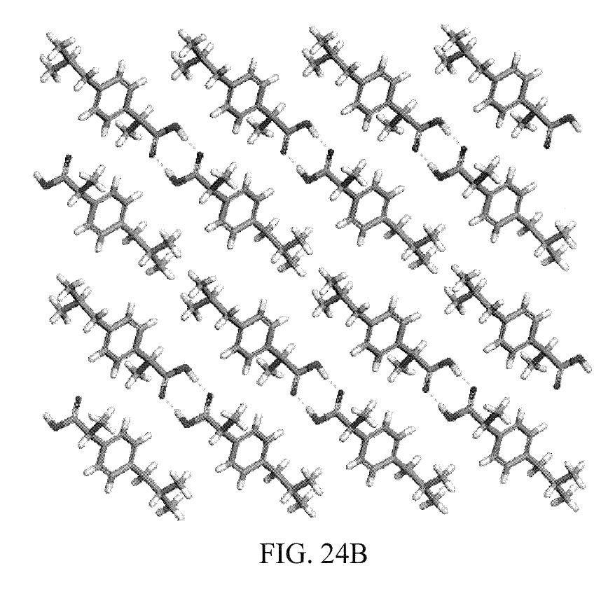

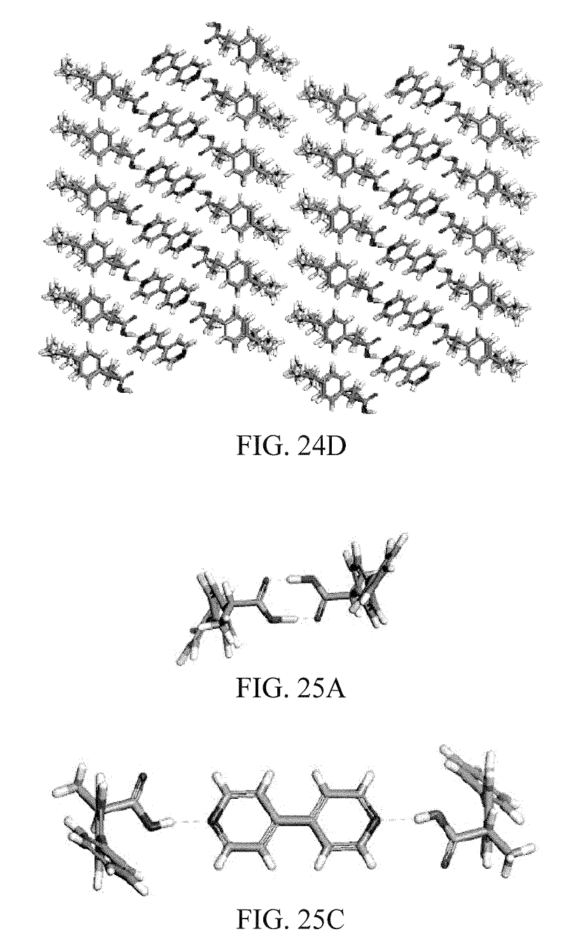

50 mg (0.242 mmol) racemic ibuprofen and 18 mg (0.0960 mmol) 4,4′-bipyridine were dissolved in 5 mL acetone. Slow evaporation of the solvent yielded colorless needles of a 2:1 ibuprofen:4,4′-bipyridine co-crystal, as shown in FIGS. 24A-D.

Crystal data: (Bruker SMART-APEX CCD Diffractometer), C₃₆H₄₄N₂O₄, M=568.73, triclinic, space group P-1; a=5.759(3), b=11.683(6), c=24.705(11) Å, α=93.674(11), β=90.880(10), γ=104.045(7)°, U=1608.3(13) Å³, T=200(2) K, Z=2, μ(Mo—Kα)=0.076 mm⁻¹, D_{c}=1.174 Mg/m³, λ=0.71073 Å, F(000)=612, 2θₘₐₓ=23.29°; 5208 reflections measured, 3362 unique (Rᵢₙₜ=0.0826). Final residuals for 399 parameters were R₁=0.0964, wR₂=0.2510 for I>2σ(I), and R₁=0.1775, wR₂=0.2987 for all 3362 data.

Crystal packing: The co-crystal contains ibuprofen:bipyridine heterodimers, sustained by two hydrogen bonded carboxylic acidpyridine supramolecular synthons, arranged in a herringbone motif that packs in the space group P-1. The heterodimer is an extended version of the homodimer and packs to form a two-dimensional network sustained by π-π stacking of the bipyridine and phenyl groups of the ibuprofen and hydrophobic interactions from the ibuprofen tails.

Infrared Spectroscopy: (Nicolet Avatar 320 FTIR). Analysis observed stretching of aromatic C—H at 2899 cm⁻¹; N—H bending and scissoring at 1886 cm₋₁; C═O stretching at 1679 cm⁻¹; C—H out-of-plane bending for both 4,4′-bipyridine and ibuprofen at 808 cm⁻¹ and 628 cm⁻¹.

Differential Scanning Calorimetry: (TA Instruments 2920 DSC), 64.85 degrees C. (endotherm) and 118.79 degrees C. (endotherm); m.p.=113-120 degrees C. (MEL-TEMP); (ibuprofen m.p.=75-77 degrees C., 4,4′-bipyridine m.p.=111-114 degrees C.). Thermogravimetric Analysis: (TA Instruments 2950 Hi-Resolution TGA), 13.28% weight loss between room temperature and 100.02 degrees C. immediately followed by complete decomposition.

Powder x-ray diffraction: (Rigaku Miniflex Diffractometer using Cu Kα (λ=1.540562), 30 kV, 15 mA). The powder data were collected over an angular range of 3° to 40° 2θ in continuous scan mode using a step size of 0.02° 2θ and a scan speed of 2.0°/minute. PXRD derived from the single crystal data, experimental (calculated): 3.4 (3.6); 6.9 (7.2); 10.4 (10.8); 17.3 (17.5); 19.1 (19.7).

### Example 21

### Flurbiprofen:4,4′-bipyridine (2:1 Stoichiometry)

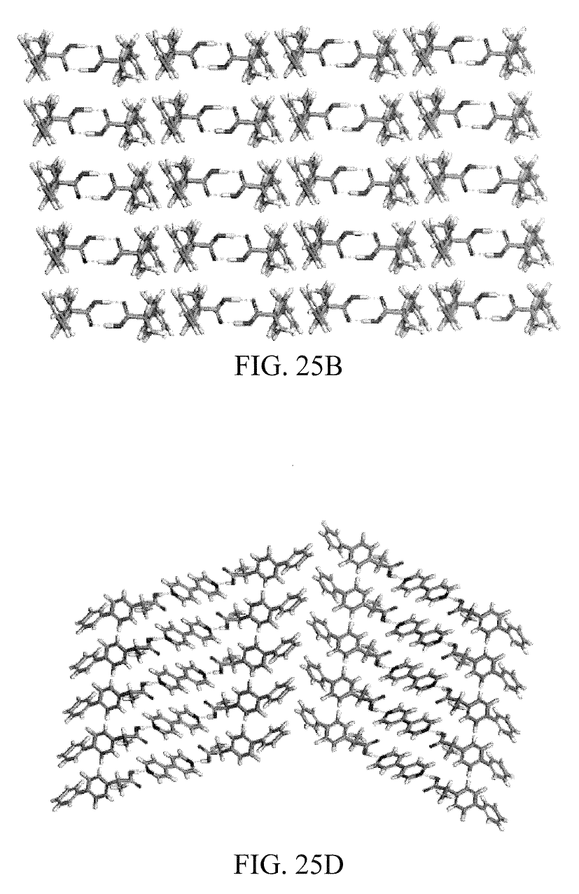

50 mg (0.2046 mmol) flurbiprofen and 15 mg (0.0960 mmol) 4,4′-bipyridine were dissolved in 3 mL acetone. Slow evaporation of the solvent yielded colorless needles of a 2:1 flurbiprofen:4,4′-bipyridine co-crystal, as shown in FIGS. 25A-D.

Crystal data: (Bruker SMART-APEX CCD Diffractometer), C₄₀H₃₄F₂N₂O₄, M=644.69, monoclinic P2₁/n; a=5.860(4), b=47.49(3), c=5.928(4) Å, β=107.382 (8)°, U=1574.3(19) Å³, T=200(2) K, Z=2, μ(Mo—Kα)=0.096 mm⁻¹, D_{c}=1.360 Mg/m³, λ=0.71073 Å, F(000)=676, 2θₘₐₓ=21.69°; 4246 reflections measured, 1634 unique (Rᵢₙₜ=0.0677). Final residuals for 226 parameters were R₁=0.0908, wR₂=0.2065 for I>2σ(I), and R₁=0.1084, wR₂=0.2209 for all 1634 data.

Crystal packing: The co-crystal contains flurbiprofen:bipyridine heterodimers, sustained by two hydrogen bonded carboxylic acidpyridine supramolecular synthon, arranged in a herringbone motif that packs in the space group P2₁/n. The heterodimer is an extended version of the homodimer and packs to form a two-dimensional network sustained by n-t stacking and hydrophobic interactions of the bipyridine and phenyl groups of the flurbiprofen.

Infrared Spectroscopy: (Nicolet Avatar 320 FTIR), aromatic C—H stretching at 3057 cm⁻¹ and 2981 cm⁻¹; N—H bending and scissoring at 1886 cm⁻¹; C═O stretching at 1690 cm⁻¹; C═C and C═N ring stretching at 1418 cm⁻¹.

Differential Scanning Calorimetry: (TA Instruments 2920 DSC), 162.47 degrees C. (endotherm); m.p.=155-160 degrees C. (MEL-TEMP); (flurbiprofen m.p.=110-111 degrees C., 4,4′-bipyridine m.p.=111-114 degrees C.).

Thermogravimetric Analysis: (TA Instruments 2950 Hi-Resolution TGA), 30.93% weight loss starting at 31.13 degrees C. and a 46.26% weight loss starting at 168.74 degrees C. followed by complete decomposition.

Powder x-ray diffraction: (Rigaku Miniflex Diffractometer using Cu Kα (λ=1.540562), 30 kV, 15 mA), the powder data were collected over an angular range of 3° □ to 40° 2θ in continuous scan mode using a step size of 0.02° 2θ and a scan speed of 2.0°/minute. PXRD derived from the single crystal data: experimental (calculated): 16.8 (16.8); 17.1 (17.5); 18.1 (18.4); 19.0 (19.0); 20.0 (20.4); 21.3 (21.7); 22.7 (23.0); 25.0 (25.6); 26.0 (26.1); 26.0 (26.6); 26.1 (27.5); 28.2 (28.7); 29.1 (29.7).

### Example 22

### Flurbiprofen:trans-1,2-bis(4-pyridyl)ethylene (2:1 Stoichiometry)

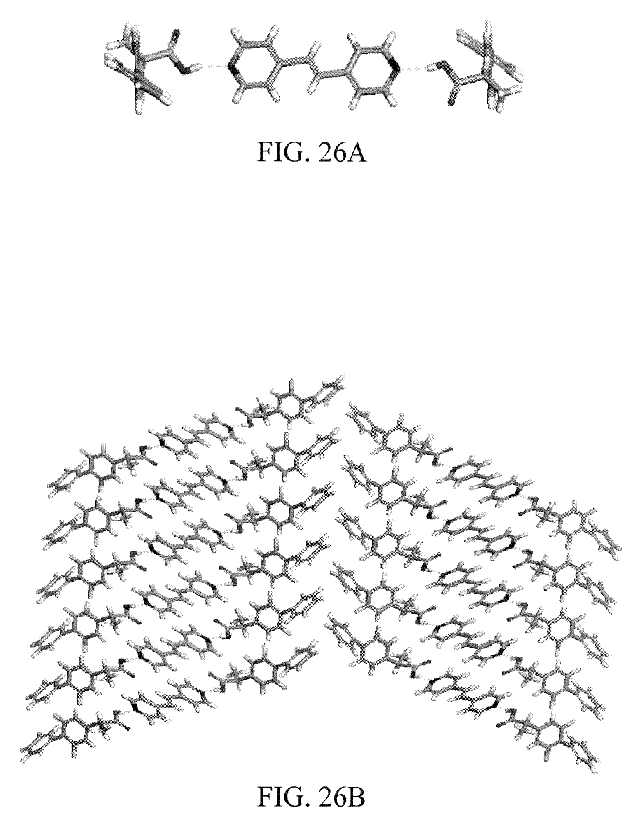

25 mg (0.1023 mmol) flurbiprofen and 10 mg (0.0548 mmol) trans-1,2-bis(4-pyridyl)ethylene were dissolved in 3 mL acetone. Slow evaporation of the solvent yielded colorless needles of a 2:1 flurbiprofen:1,2-bis(4-pyridyl)ethylene co-crystal, as shown in FIGS. 26A-B.

Crystal data: (Bruker SMART-APEX CCD Diffractometer), C₄₂H₃₆F₂N₂O₄, M=670.73, monoclinic P2₁/n; a=5.8697(9), b=47.357(7), c=6.3587(10) Å, β=109.492(3)°, U=1666.2(4) Å³, T=200(2) K, Z=2, μ(Mo—Kα)=0.093 mm⁻¹, D_{c}=1.337 Mg/m³, λ=0.71073 Å, F(000)=704, 2θₘₐₓ=21.69°, 6977 reflections measured, 2383 unique (Rᵢₙₜ=0.0383). Final residuals for 238 parameters were R₁=0.0686, wR₂=0.1395 for I>2σ(I), and R₁=0.1403, wR₂=0.1709 for all 2383 data.

Crystal packing: The co-crystal contains flurbiprofen:1,2-bis(4-pyridyl)ethylene heterodimers, sustained by two hydrogen bonded carboxylic acid-pyridine supramolecular synthons, arranged in a herringbone motif that packs in the space group P2₁/n. The heterodimer from 1,2-bis(4-pyridyl)ethylene further extends the homodimer relative to example 21 and packs to form a two-dimensional network sustained by π-π stacking and hydrophobic interactions of the bipyridine and phenyl groups of the flurbiprofen.

Infrared Spectroscopy: (Nicolet Avatar 320 FTIR), aromatic C—H stretching at 2927 cm⁻¹ and 2850 cm⁻¹; N—H bending and scissoring at 1875 cm⁻¹; C═O stretching at 1707 cm⁻¹; C═C and C═N ring stretching at 1483 cm⁻¹.

Differential Scanning Calorimetry: (TA Instruments 2920 DSC), 100.01 degrees C., 125.59 degrees C. and 163.54 degrees C. (endotherms); m.p.=153-158 degrees C. (MEL-TEMP); (flurbiprofen m.p.=110-111 degrees C., trans-1,2-bis(4-pyridyl)ethylene m.p.=150-153 degrees C.).

Thermogravimetric Analysis: (TA Instruments 2950 Hi-Resolution TGA), 91.79% weight loss starting at 133.18 degrees C. followed by complete decomposition. Powder x-ray diffraction: (Rigaku Miniflex Diffractometer using Cu Kα (λ=1.540562), 30 kV, 15 mA), the powder data were collected over an angular range of 3° to 40° 2θ in continuous scan mode using a step size of 0.02° 2θ and a scan speed of 2.0°/minute. PXRD derived from the single crystal data, experimental (calculated): 3.6 (3.7); 17.3 (17.7); 18.1 (18.6); 18.4 (18.6); 19.1 (19.3); 22.3 (22.5); 23.8 (23.9); 25.9 (26.4); 28.1 (28.5).

### Example 23

### Carbamazepine:p-Phthalaldehyde (2:1 Stoichiometry)

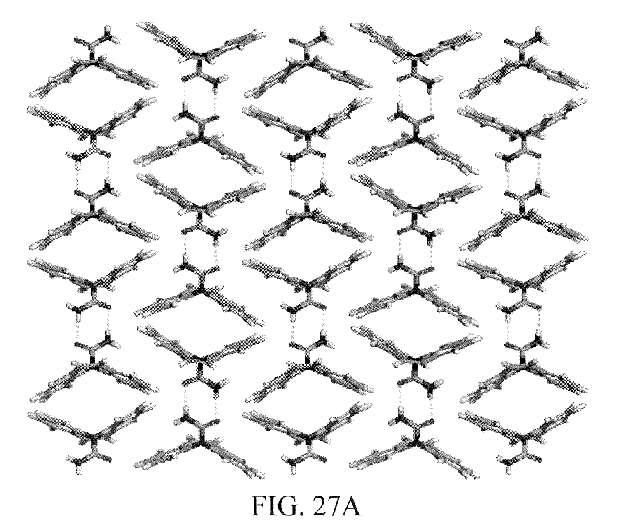

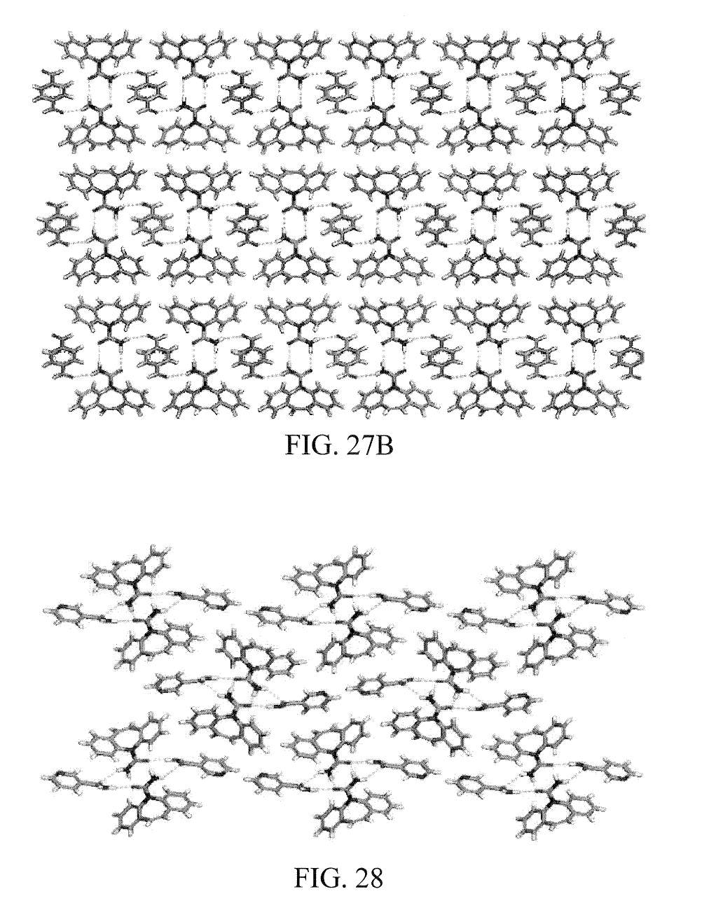

25 mg (0.1058 mmol) carbamazepine and 7 mg (0.0521 mmol)ₚ-phthalaldehyde were dissolved in approximately 3 mL methanol. Slow evaporation of the solvent yielded colorless needles of a 2:1 carbamazepine:p-phthalaldehyde co-crystal, as shown in FIGS. 27A-B.

Crystal data: (Bruker SMART-APEX CCD Diffractometer), C₃₈H₃₀N₄O₄, M=606.66, monoclinic C2/c; a=29.191(16), b=4.962(3), c=20.316(11) Å, β=92.105(8)°, U=2941(3) Å³, T=200(2) K, Z=4, μ(Mo—Kα)=0.090 mm⁻¹, D_{c}=1.370 Mg/m³, λ=0.71073 Å, F(000)=1272, 2θ=43.66°, 3831 reflections measured, 1559 unique (Rᵢₙₜ=0.0510). Final residuals for 268 parameters were R₁=0.0332, wR₂=0.0801 for I>2σ(I), and R₁=0.0403, wR₂=0.0831 for all 1559 data.

Crystal packing: The co-crystals contain hydrogen bonded carboxamide homodimers that crystallize in the space group C2/c. The 1° amines of the homodimer are bifurcated to the carbonyl of the p-phthalaldehyde forming a chain with an adjacent homodimer. The chains pack in a crinkled tape motif sustained by π-π interactions between phenyl rings of the carbamazepine.

Infrared Spectroscopy: (Nicolet Avatar 320 FTIR). The 1° amine unsymmetrical and symmetrical stretching was shifted down to 3418 cm⁻¹; aliphatic aldehyde and 1° amide C═O stretching was shifted up to 1690 cm⁻¹; N—H in-plane bending at 1669 cm⁻¹; C—H aldehyde stretching at 2861 cm⁻¹ and H—C═O bending at 1391 cm⁻¹.

Differential Scanning Calorimetry: (TA Instruments 2920 DSC), 128.46 degrees C. (endotherm), m.p.=121-124 degrees C. (MEL-TEMP), (carbamazepine m.p.=190.2 degrees C., p-phthalaldehyde m.p.=116 degrees C.).

Thermogravimetric Analysis: (TA Instruments 2950 Hi-Resolution TGA), 17.66% weight loss starting at 30.33 degrees C. then a 17.57% weight loss starting at 100.14 degrees C. followed by complete decomposition.

Powder x-ray diffraction: (Rigaku Miniflex Diffractometer using Cu Kα (λ=1.540562), 30 kV, 15 mA). The powder data were collected over an angular range of 3° to 40° 2θ in continuous scan mode using a step size of 0.02° 2θ and a scan speed of 2.0°/minute. PXRD derived from the single crystal data, experimental (calculated): 8.5 (8.7); 10.6 (10.8); 11.9 (12.1); 14.4 (14.7) 15.1 (15.2); 18.0 (18.1); 18.5 (18.2); 19.8 (18.7); 23.7 (24.0); 24.2 (24.2); 26.4 (26.7); 27.6 (27.9); 27.8 (28.2); 28.7 (29.1); 29.3 (29.6); 29.4 (29.8).

### Example 24

### Carbamazepine:nicotinamide (1:1 Stoichiometry)

25 mg (0.1058 mmol) carbamazepine and 12 mg (0.0982 mmol) nicotinamide were dissolved in 4 mL of DMSO, methanol or ethanol. Slow evaporation of the solvent yielded colorless needles of a 1:1 carbamazepine:nicotinamide co-crystal, as shown in FIG. 28. Using a separate method, 25 mg (0.1058 mmol) carbamazepine and 12 mg (0.0982 mmol) nicotinamide were ground together with mortar and pestle. The solid was determined to be 1:1 carbamazepine:nicotinamide microcrystals (PXRD).

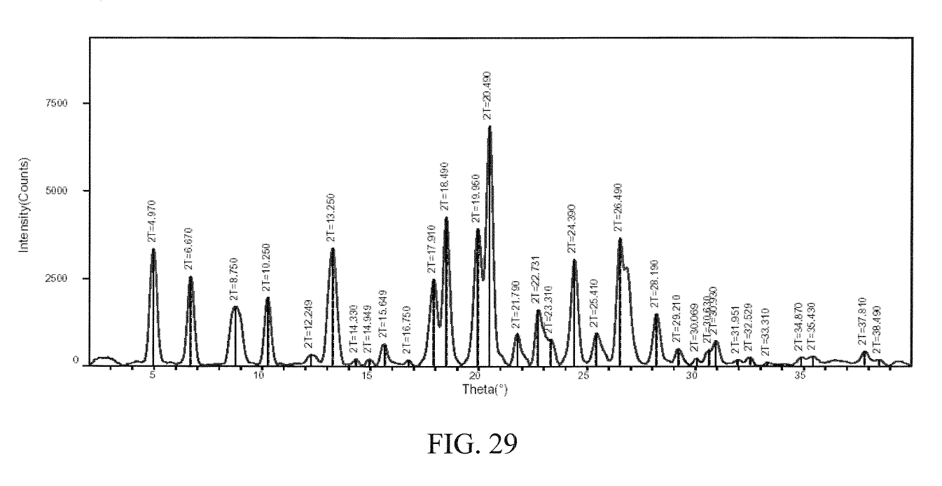

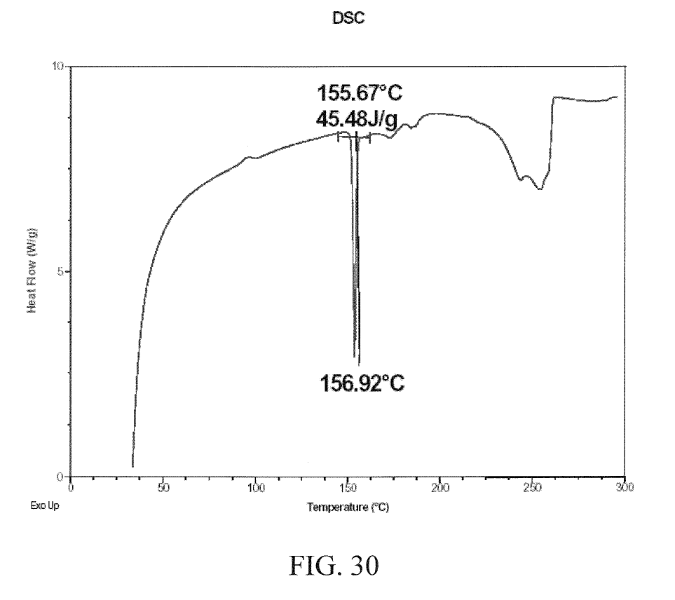

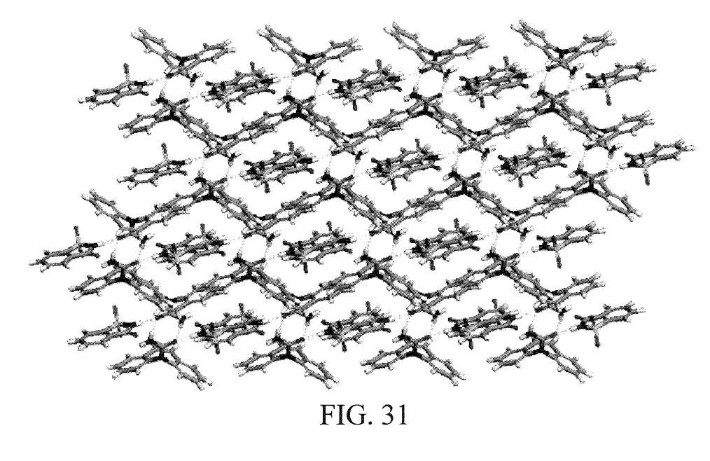

1:1 carbamazepine:nicotinamide co-crystals were also prepared via another method. A 12-block experiment was designed with 12 solvents. (A block is a receiving plate, which can be an industry standard 96 well, 384 well, or 1536 well format, or a custom format.) 1152 crystallization experiments were carried out using the CrystalMax™ platform. The co-crystal was obtained from samples containing toluene, acetone, or isopropyl acetate. The resulting co-crystal was characterized by PXRD and DSC and these data are shown in FIGS. 29 and 30, respectively. The co-crystals prepared from toluene, acetone, or isopropyl acetate may contain impurities such as carbamazepine in free form due to incomplete purification.

Crystal data: (Bruker SMART-APEX CCD Diffractometer), C₂₁H₁₈N₄O₂, M=358.39, monoclinic P2₁/n; a=5.0961(8), b=17.595(3), c=19.647(3) Å, β=90.917(3)°, U=1761.5(5) Å³, T=200(2) K, Z=4, μ(Mo—Kα)=0.090 mm⁻¹, D_{c}=1.351 Mg/m³, λ=0.71073 Å, F(000)=752, 2θₘₐₓ=56.60°, 10919 reflections measured, 4041 unique (Rᵢₙₜ=0.0514). Final residuals for 248 parameters were R₁=0.0732, wR₂=0.1268 for I>2σ(I), and R₁=0.1161, wR₂=0.1430 for all 4041 data.

Crystal packing: The co-crystals contain hydrogen bonded carboxamide homodimers. The 1° amines are bifurcated to the carbonyl of the nicotinamide on each side of the dimer. The 1° amines of each nicotinamide are hydrogen bonded to the carbonyl of the adjoining dimer. The dimers form chains with π-π interactions from the phenyl groups of the carbamazepine.

Infrared Spectroscopy: (Nicolet Avatar 320 FTIR), unsymmetrical and symmetrical stretching shifts down to 3443 cm⁻¹ and 3388 cm⁻¹ accounting for 1° amines; 1° amide C═O stretching at 1690 cm⁻¹; N—H in-plane bending at 1614 cm⁻¹; C═C stretching shifted down to 1579 cm⁻¹; aromatic H's from 800 cm⁻¹ to 500 cm⁻¹ are present.

Differential Scanning Calorimetry: (TA Instruments 2920 DSC), 74.49 degrees C. (endotherm) and 159.05 degrees C. (endotherm), m.p.=153-158 degrees C. (MEL-TEMP), (carbamazepine m.p.=190.2 degrees C., nicotinamide m.p.=150-160 degrees C.).

Thermogravimetric Analysis: (TA Instruments 2950 Hi-Resolution TGA), 57.94% weight loss starting at 205.43 degrees C. followed by complete decomposition.

Powder x-ray diffraction: (Rigaku Miniflex Diffractometer using Cu Kα (λ=1.540562), 30 kV, 15 mA). The powder data were collected over an angular range of 3° to 40° 2θ in continuous scan mode using a step size of 0.02° 2θ and a scan speed of 2.0°/minute. PXRD: Showed analogous peaks to the simulated PXRD derived from the single crystal data. PXRD analysis experimental (calculated): 6.5 (6.7); 8.8 (9.0); 10.1 (10.3); 13.2 (13.5); 15.6 (15.8); 17.7 (17.9); 17.8 (18.1); 18.3 (18.6); 19.8 (20.1); 20.4 (20.7); 21.6 (N/A); 22.6 (22.8); 22.9 (23.2); 26.4 (26.7); 26.7 (27.0); 28.0 (28.4).

### Example 25

### Carbamazepine:saccharin (1:1 Stoichiometry)

25 mg (0.1058 mmol) carbamazepine and 19 mg (0.1037 mmol) saccharin were dissolved in approximately 4 mL ethanol. Slow evaporation of the solvent yielded colorless needles of a 1:1 carbamazepine:saccharin co-crystal, as shown in FIG. 48. Solubility measurements indicate that this co-crystal of carbamazepine has improved solubility over previously known forms of carbamazepine (e.g., increased molar solubility and longer solubility in aqueous solutions).

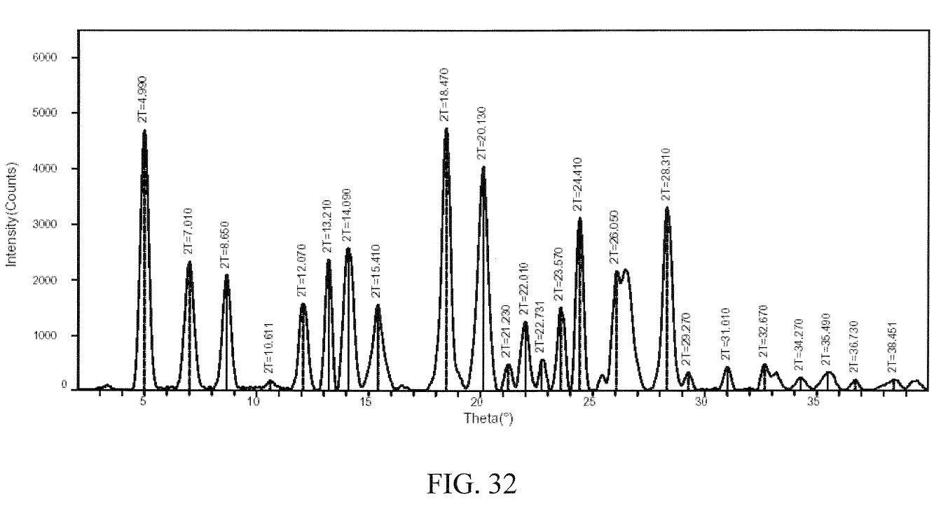

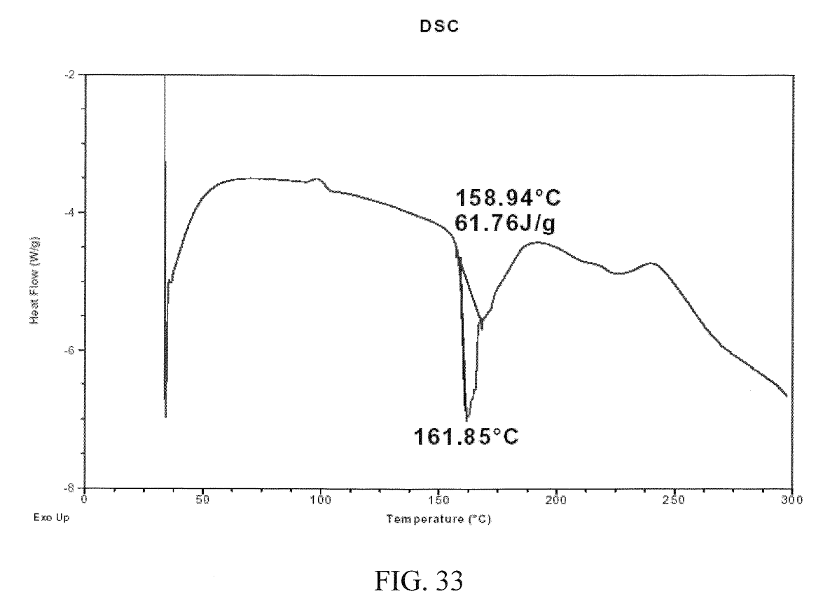

1:1 carbamazepine:saccharin co-crystals were also prepared via another method. A 12-block experiment was designed with 12 solvents. (A block is a receiving plate, which can be an industry standard 96 well, 384 well, or 1536 well format, or a custom format.) 1152 crystallization experiments were carried out using the CrystalMax™ platform. The carbamazepine:saccharin co-crystal was obtained from a mixture of isopropyl acetate and heptane. The resulting co-crystal was characterized by PXRD and DSC and these data are shown in FIGS. 32 and 33, respectively. The co-crystal prepared from a mixture of isopropyl acetate and heptane may contain impurities such as carbamazepine in free form due to incomplete purification.

Crystal data: (Bruker SMART-APEX CCD Diffractometer), C₂₂H₁₇N₃O₄S, M=419.45, triclinic P-1; a=7.5140(11), b=10.4538(15), c=12.6826(18) Å, α=83.642(2)°, β=85.697(2)°, γ=75.411(2)°, U=957.0(2) Å³, T=200(2) K, Z=2, μ(Mo—Kα)=0.206 mm⁻¹, D_{c}=1.456 Mg/m³, λ=0.71073 Å, F(000)=436, 2θₘₐₓ=56.20°; 8426 reflections measured, 4372 unique (Rᵢₙₜ=0.0305). Final residuals for 283 parameters were R₁=0.0458, wR₂=0.1142 for I>2σ(I), and R₁=0.0562, wR₂=0.1204 for all 4372 data.

Crystal packing: The co-crystals contain hydrogen bonded carboxamide homodimers. The 2° amines of the saccharin are hydrogen bonded to the carbonyl of the carbamazepine on each side forming a tetramer. The crystal has a space group of P-1 with π-π interactions between the phenyl groups of the carbamazepine and the saccharin phenyl groups. Infrared Spectroscopy: (Nicolet Avatar 320 FTIR), unsymmetrical and symmetrical stretching shifts up to 3495 cm⁻¹ accounting for 1° amines; C═O aliphatic stretching was shifted up to 1726 cm⁻¹; N—H in-plane bending at 1649 cm⁻¹; C═C stretching shifted down to 1561 cm⁻¹; (O═S═O) sulfonyl peak at 1330 cm⁻¹ C—N aliphatic stretching 1175 cm⁻¹.

Differential Scanning Calorimetry: (TA Instruments 2920 DSC), 75.31 degrees C. (endotherm) and 177.32 degrees C. (endotherm), m.p.=148-155 degrees C. (MEL-TEMP); (carbamazepine m.p.=190.2 degrees C., saccharin m.p.=228.8 degrees C.).

Thermogravimetric Analysis: (TA Instruments 2950 Hi-Resolution TGA), 3.342% weight loss starting at 67.03 degrees C. and a 55.09% weight loss starting at 118.71 degrees C. followed by complete decomposition.

Powder x-ray diffraction: (Rigaku Miniflex Diffractometer using Cu Kα (λ=1.540562), 30 kV, 15 mA). The powder data were collected over an angular range of 3° to 40° 2θ in continuous scan mode using a step size of 0.02° 2θ and a scan speed of 2.0°/minute. PXRD derived from the single crystal data, experimental (calculated): 6.9 (7.0); 12.2 (12.2); 13.6 (13.8); 14.0 (14.1); 14.1 (14.4); 15.3 (15.6); 15.9 (15.9); 18.1 (18.2); 18.7 (18.8); 20.2 (20.3); 21.3 (21.5); 23.7 (23.9); 26.3 (26.4); 28.3 (28.3).

### Example 26

### Carbamazepine:2,6-pyridinedicarboxylic acid (1:1 Stoichiometry)

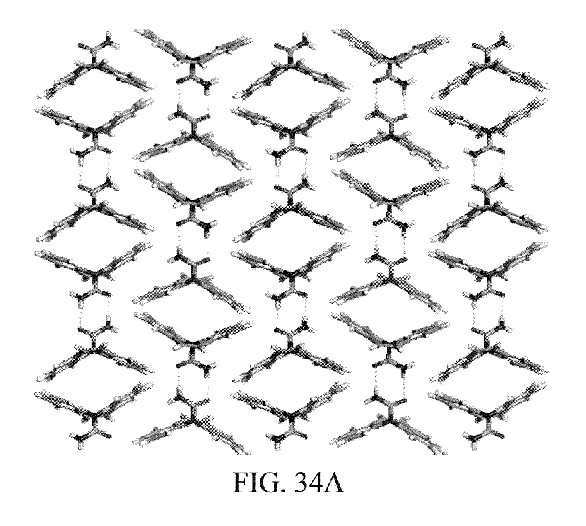

36 mg (0.1524 mmol) carbamazepine and 26 mg (0.1556 mmol) 2,6-pyridinedicarboxylic acid were dissolved in approximately 2 mL ethanol. Slow evaporation of the solvent yielded clear needles of a 1:1 carbamazepine:2,6-pyridinedicarboxylic acid co-crystal, as shown in FIGS. 34A-B.

Crystal data: (Bruker SMART-APEX CCD Diffractometer). C₂₂H₁₇N₃O₅. M=403.39, orthorhombic P2(1)2(1)2(1); a=7.2122, b=14.6491, c=17.5864 Å, α=90°, β=90°, γ=90°, U=1858.0(2) Å³, T=100 K, Z=4, μ(MO-Kα)=0.104 mm⁻¹, D_{c}-1.442 Mg/m³, λ=0.71073 Å, F(000)840, 2θₘₐₓ=28.3. 16641 reflections measured, 4466 unique (Rᵢₙₜ=0.093). Final residuals for 271 parameters were R₁=0.0425 and wR₂=0.0944 for I>2σ(I).

Crystal packing: Each hydrogen on the carbamazepine 1° amine is hydrogen bonded to a carbonyl group of a different 2,6-pyridinedicarboxylic acid moiety. The carbonyl of the carbamazepine carboxamide is hydrogen bonded to two hydroxide groups of one 2,6-pyridinedicarboxylic acid moiety.

Infrared Spectroscopy: (Nicolet Avatar 320 FTIR). 3439 cm⁻¹, (N—H stretch, 1° amine, carbamazepine); 1734 cm⁻¹, (C═O); 1649 cm⁻¹, (C═C).

Melting Point: 214-216 degrees C. (MEL-TEMP). (carbamazepine m.p.=191-192 degrees C., 2,6-pyridinedicarboxylic acid m.p.=248-250 degrees C.).

Thermogravimetric Analysis: (TA Instruments 2950 Hi-Resolution TGA). 69% weight loss starting at 215 degrees C. and a 17% weight loss starting at 392 degrees C. followed by complete decomposition.

### Example 27

### Carbamazepine:5-nitroisophthalic acid (1:1 Stoichiometry)

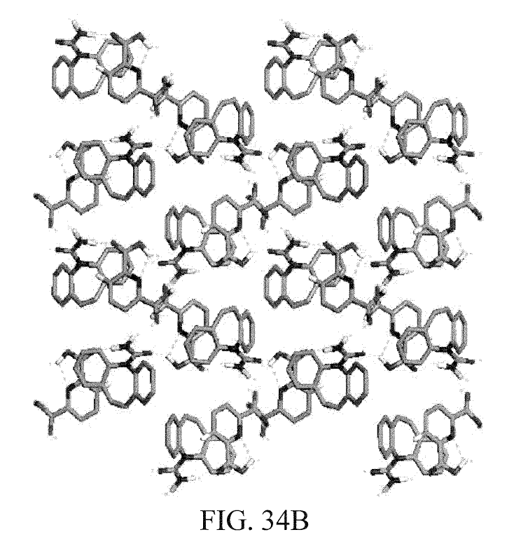

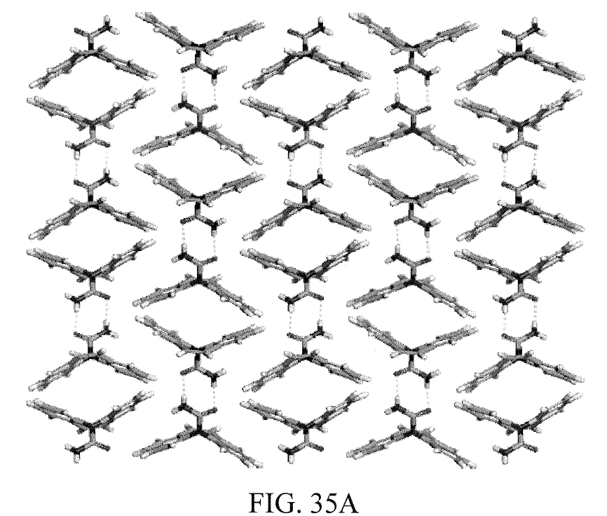

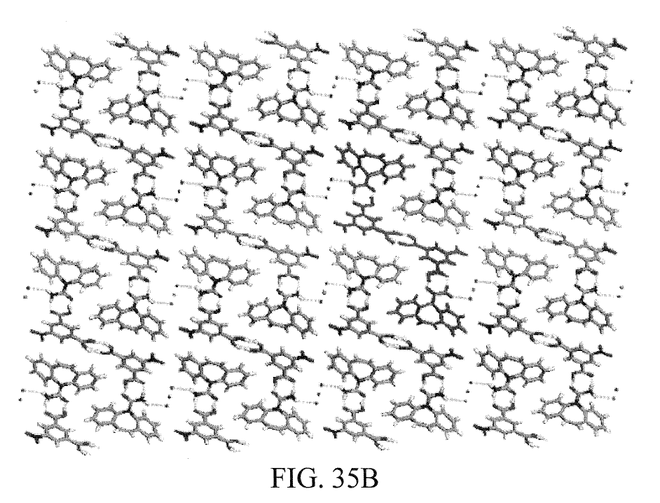

40 mg (0.1693 mmol) carbamazepine and 30 mg (0.1421 mmol) 5-nitroisophthalic acid were dissolved in approximately 3 mL methanol or ethanol. Slow evaporation of the solvent yielded yellow needles of a 1:1 carbamazepine:5-nitroisophthalic acid co-crystal, as shown in FIGS. 35A-B.

Crystal data: (Bruker SMART-APEX CCD Diffractometer). monoclinic C2/c; a=34.355(8), b=5.3795(13), c=23.654(6) Å, α=90°, β=93.952(6)°, γ=90°, U=4361.2(18) Å³, T=200(2) K, Z=4, μ(MO-Kα)=0.110 mm⁻¹, D_{c}=1.439 Mg/m³, λ=0.71073 Å, F(000)1968, 2θₘₐₓ=26.43°. 11581 reflections measured, 4459 unique (Rᵢₙₜ=0.0611). Final residuals for 311 parameters were R₁=0.0725, wR₂=0.1801 for I>2σ(I), and R₁=0.1441, wR₂=0.1204 for all 4459 data.

Crystal packing: The co-crystals are sustained by hydrogen bonded carboxylic acid homodimers between the two 5-nitroisophthalic acid moieties and hydrogen bonded carboxy-amide heterodimers between the carbamazepine and 5-nitroisophthalic acid moiety. There is solvent hydrogen bonded to an additional N—H donor from the carbamazepine moiety. Infrared Spectroscopy: (Nicolet Avatar 320 FTIR). 3470 cm⁻¹, (N—H stretch, 1° amine, carbamazepine); 3178 cm⁻¹, (C—H stretch, alkene); 1688 cm⁻¹, (C═O); 1602 cm⁻¹, (C═C). Differential Scanning Calorimetry: (TA Instruments 2920 DSC). 190.51 degrees C. (endotherm). m.p.=NA (decomposes at 197-200 degrees C.) (MEL-TEMP). (carbamazepine m.p.=191-192 degrees C., 5-nitroisophthalic acid m.p.=260-261 degrees C.).

Thermogravimetric Analysis: (TA Instruments 2950 Hi-Resolution TGA). 32.02% weight loss starting at 202 degrees C., a 12.12% weight loss starting at 224 degrees C. and a 17.94% weight loss starting at 285 degrees C. followed by complete decomposition.

Powder x-ray diffraction: (Rigaku Miniflex Diffractometer using CuKα (λ=1.540562), 30 kV, 15 mA). The powder data were collected over an angular range of 3 to 40 2 in continuous scan mode using a step size of 0.02 2 and a scan speed of 2.0/min. PXRD: Showed analogous peaks to the simulated PXRD derived from the single crystal data. PXRD analysis experimental (calculated): 10.138 (10.283), 15.291 (15.607), 17.438 (17.791), 21.166 (21.685), 31.407 (31.738), 32.650 (32.729).

### Example 28

### Carbamazepine:1,3,5,7-adamantane tetracarboxylic acid (2:1 Stoichiometry)

15 mg (0.1524 mmol) carbamazepine and 20 mg (0.1556 mmol) 1,3,5,7-adamantanetetracarboxylic acid were dissolved in approximately 1 mL methanol or 1 mL ethanol. Slow evaporation of the solvent yields clear plates of a 2:1 carbamazepine:1,3,5,7-adamantanetetracarboxylic acid co-crystal, as shown in FIGS. 36A-B.

Crystal data: (Bruker SMART-APEX CCD Diffractometer). C₄₄H₄₀N₄O₁₀, M=784.80, monoclinic C2/c; a=18.388(4), b=12.682(3), c=16.429(3) Å, β=100.491(6)°, U=3767.1(14) Å³, T=100(2) K, Z=4, μ(MO-Kα)=0.099 mm⁻¹, D_{c}-1.384 Mg/m³, λ=0.71073 Å, F(000)1648, 2θₘₐₓ=28.20°. 16499 reflections measured, 4481 unique (Rᵢₙₜ=0.052). Final residuals for 263 parameters were R₁=0.0433 and wR₂=0.0913 for I>2σ(I).

Crystal packing: The co-crystals form a single 3D network of four tetrahedron, linked by square planes similar to the PtS topology. The crystals are sustained by hydrogen bonding.

Infrared Spectroscopy: (Nicolet Avatar 320 FTIR). 3431 cm⁻¹, (N—H stretch, 1° amine, carbamazepine); 3123 cm⁻¹, (C—H stretch, alkene); 1723 cm⁻¹, (C═O); 1649 cm⁻¹, (C═C). Melting Point: (MEL-TEMP). 258-260 degrees C. (carbamazepine m.p.=191-192 degrees C., adamantanetetracarboxylic acid m.p.=>390 degrees C.).

Thermogravimetric Analysis: (TA Instruments 2950 Hi-Resolution TGA). 9% weight loss starting at 189 degrees C., a 52% weight loss starting at 251 degrees C. and a 31% weight loss starting at 374 degrees C. followed by complete decomposition.

### Example 29

### Carbamazepine:benzoquinone (1:1 Stoichiometry)

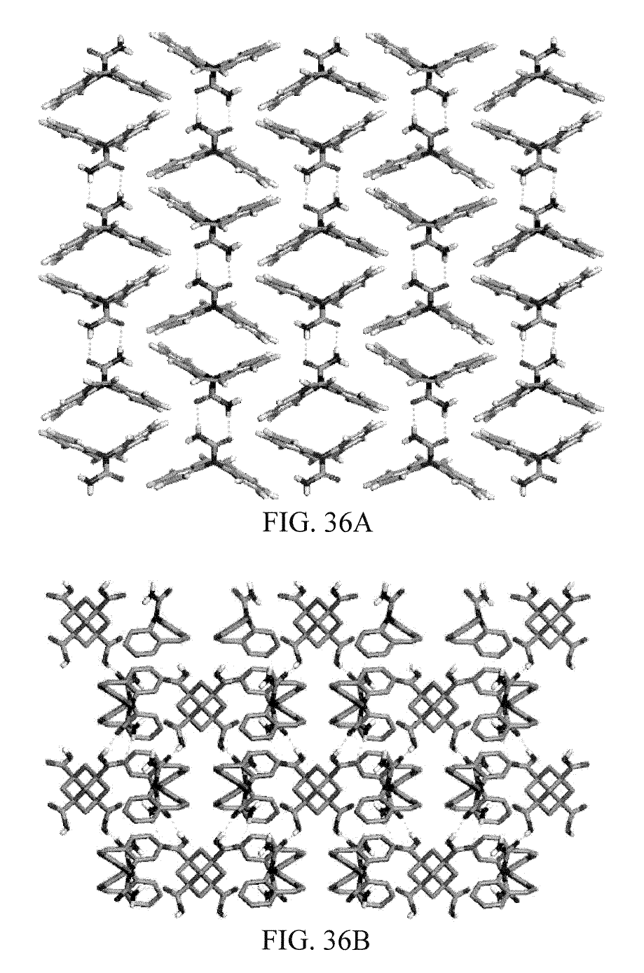

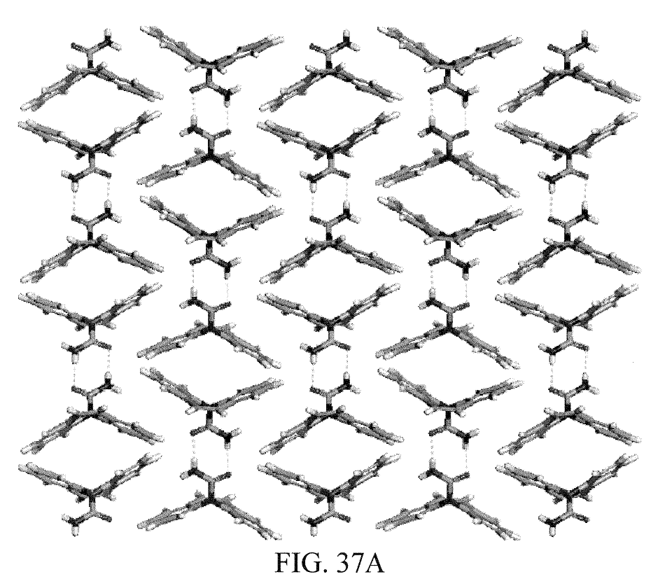

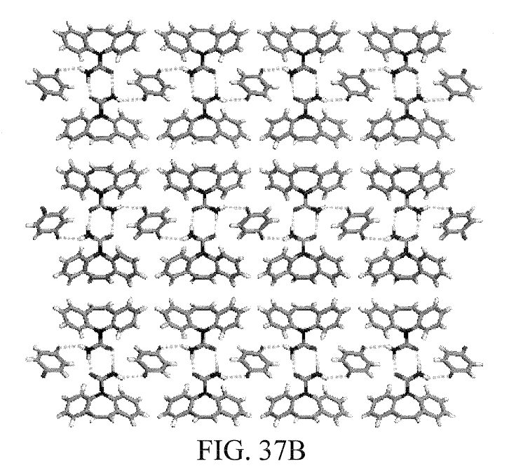

25 mg (0.1058 mmol) carbamazepine and 11 mg (0.1018 mmol) benzoquinone was dissolved in 2 mL methanol or THF. Slow evaporation of the solvent produced an average yield of yellow crystals of a 1:1 carbamazepine:benzoquinone co-crystal, as shown in FIGS. 37A-B.

Crystal data: (Bruker SMART-APEX CCD Diffractometer). C₂₁H₁₆N₂O₃, M=344.36, monoclinic P2(1)/c; a=10.3335(18), b=27.611(5), c=4.9960(9) Å, β=102.275(3)°, U=1392.9(4) Å³, T=100(2) K, Z=3, D_{c}=1.232 Mg/m³, μ(MO-Kα)=0.084 mm⁻¹, λ=0.71073 Å, F(000)540, 2θₘₐₓ=28.24°. 8392 reflections measured, 3223 unique (Rᵢₙₜ=0.1136). Final residuals for 199 parameters were R₁=0.0545 and wR₂=0.1358 for I>2σ(I), and R₁=0.0659 and wR₂=0.1427 for all 3223 data.

Crystal packing: The co-crystals contain hydrogen bonded carboxamide homodimers. Each 1° amine on the carbamazepine is bifurcated to a carbonyl group of a benzoquinone moiety. The dimers form infinite chains.

Infrared Spectroscopy: (Nicolet Avatar 320 FTIR). 3420 cm⁻¹, (N—H stretch, 1° amine, carbamazepine); 2750 cm⁻¹, (aldehyde stretch); 1672 cm⁻¹, (C═O); 1637 cm⁻¹, (C═C, carbamazepine).

Melting Point: 170 degrees C. (MEL-TEMP). (carbamazepine m.p.=191-192 degrees C., benzoquinone m.p.=115.7 degrees C.).

Thermogravimetric Analysis: (TA Instruments 2950 Hi-Resolution TGA). 20.62% weight loss starting at 168 degrees C. and a 78% weight loss starting at 223 degrees C. followed by complete decomposition.

### Example 30

### Carbamazepine:trimesic acid (1:1 Stoichiometry)

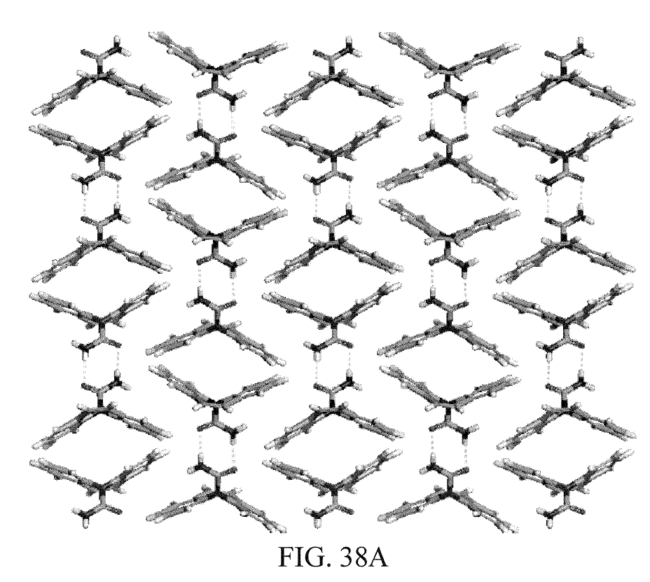

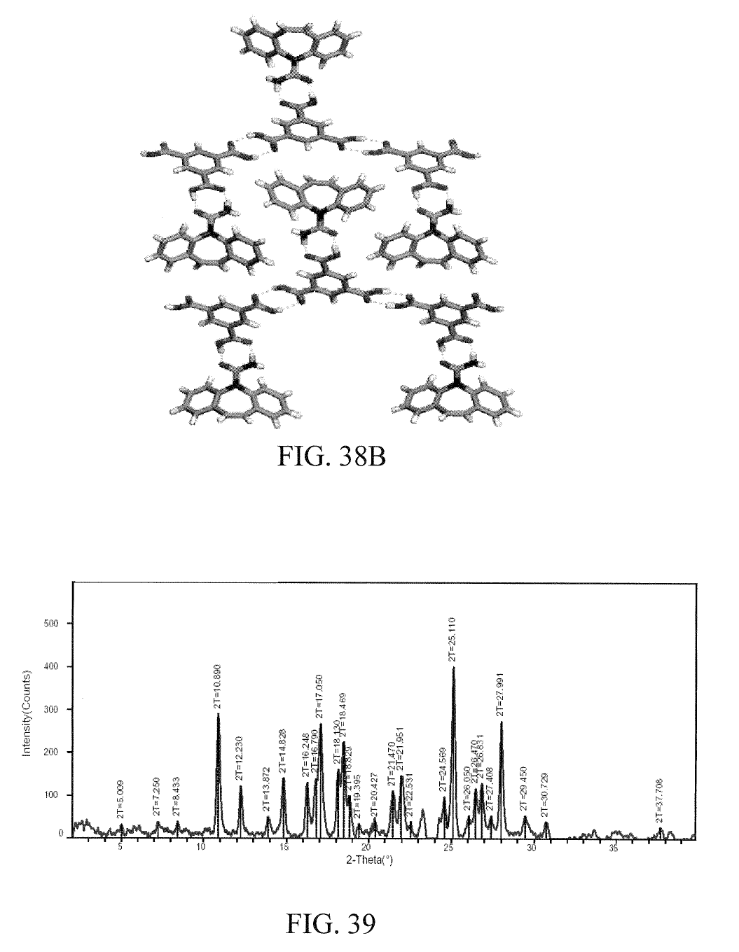

36 mg (0.1524 mmol) carbamazepine and 31 mg (0.1475 mmol) trimesic acid were dissolved in a solvent mixture of approximately 2 mL methanol and 2 mL dichloromethane. Slow evaporation of the solvent mixture yielded white starbursts of a 1:1 carbamazepine:trimesic acid co-crystal, as shown in FIGS. 38A-B.

1:1 carbamazepine:trimesic acid co-crystals were also prepared via another method. A 9-block experiment was designed with 10 solvents. 864 crystallization experiments with 8 co-crystal formers and 3 concentrations were carried out using the CrystalMax™ platform. The co-crystal was obtained from samples containing methanol. The resulting co-crystal was characterized by PXRD and the diffractogram is shown in FIG. 39.

Crystal data: (Bruker SMART-APEX CCD Diffractometer). C₂₄H₁₈N₂O₇, M=446.26, monoclinic C2/c; a=32.5312(50), b=5.2697(8), c=24.1594(37) Å, α=90°, β=98.191(3)°, γ=90°, U=4099.39(37) Å³, T=−173 K, Z=8, μ(MO-Kα)=0.110 mm⁻¹, D_{c}=1.439 Mg/m³, λ=0.71073 Å, F(000)1968, 2θₘₐₓ=26.43°. 11581 reflections measured, 4459 unique (Rᵢₙₜ=0.0611). Final residuals for 2777 parameters were R₁=0.1563, wR₂=0.1887 for I>2σ(I), and R₁=0.1441, wR₂=0.1204 for all 3601 data.

Crystal packing: The co-crystals are sustained by hydrogen bonded carboxylic acid homodimers between carbamazepine and trimesic acid moieties and hydrogen bonded carboxylic acid-amine heterodimers between two trimesic acid moieties arranged in a stacked ladder formation.

Infrared Spectroscopy: (Nicolet Avatar 320 FTIR). 3486 cm⁻¹(N—H stretch, 1° amine, carbamazepine); 1688 cm⁻¹ (C═O, 1° amide stretch, carbamazepine); 1602 cm⁻¹ (C═C, carbamazepine).

Differential Scanning Calorimetry: (TA Instruments 2920 DSC). 273 degrees C. (endotherm). m.p.=NA, decomposes at 278 degrees C. (MEL-TEMP). (carbamazepine m.p.=191-192 degrees C., trimesic acid m.p.=380 degrees C.)

Thermogravimetric Analysis: (TA Instruments 2950 Hi-Resolution TGA). 62.83% weight loss starting at 253 degrees C. and a 30.20% weight loss starting at 278 degrees C. followed by complete decomposition.

Powder x-ray diffraction: (Rigaku Miniflex Diffractometer using CuKα (λ=1.540562), 30 kV, 15 mA). The powder data were collected over an angular range of 3 to 40 degrees 2-theta in continuous scan mode using a step size of 0.02 degrees 2-theta and a scan speed of 2.0/min. PXRD analysis experimental: 10.736, 12.087, 16.857, 24.857, 27.857.

TABLE XXIV
Detailed Characterization of Co-Crystals

Celecoxib:Nicotinamide (Example 1)
PXRD: 3.77, 7.56, 9.63, 14.76, 15.21, 16.01, 17.78, 18.68, 19.31,
20.44, 21.19, 22.10
DSC: Two endothermic transitions at about 117 and 119 degrees C.
and a sharp endotherm at about 130 degrees C.
TGA: Decomposition beginning at about 150 degrees C.
Raman: 1618, 1599, 1452, 1370, 1163, 1044, 973, 796, 632, 393, 206
Celecoxib:18-Crown-6 (Example 2)
PXRD: 8.73, 11.89, 12.57, 13.13, 15.01, 16.37, 17.03, 17.75, 18.45,
20.75, 22.37, 23.11, 24.33, 24.97, 26.61, 28.15
DSC: Sharp endotherm at about 190 degrees C.
TGA: Decomposition above 200 degrees C. with a 25% weight loss
between about 190-210 degrees C.
Topiramate:18-Crown-6 (Example 3)
PXRD: 10.79, 11.07, 12.17, 13.83, 16.13, 18.03, 18.51, 18.79, 19.21,
21.43, 22.25, 24.11
DSC: Sharp endotherm at about 135 degrees C.
TGA: Rapid decomposition beginning at about 135 degrees C. and
leveling off slightly after 200 degrees C.
Raman: 2995, 2943, 1472, 1427, 1262, 849, 805, 745, 629, 280, 226
Olanzapine:Nicotinamide (Example 4)
PXRD (Form I): 4.89, 8.65, 12.51, 14.19, 15.59, 17.15, 19.71, 21.05,
23.95, 24.59, 25.53, 26.71
PXRD (Form II): 5.13, 8.65, 11.87, 14.53, 17.53, 18.09, 19.69, 24.19,
26.01 (data as received)
PXRD (Form III): 6.41, 12.85, 14.91, 18.67, 21.85, 24.37
DSC (Form I): Slightly broad endotherm at about 126 degrees C.
cis-Itraconazole:Succinic Acid (Example 5)
PXRD: 3.01, 6.01, 8.13, 9.05, 15.87, 16.17, 17.29, 24.47
DSC: Single endothermic transition at about 160 degrees C. ± 1.0
degrees C.
TGA: Less than 0.1% volatile components by weight
cis-Itraconazole:Fumaric Acid (Example 6)
PXRD: 4.61, 5.89, 9.23, 10.57, 15.51, 16.23, 16.93, 19.05, 20.79
DSC: The material had a weak endothermic transition at about 142
degrees C. and a strong endothermic transition at about 180 degrees C.
TGA: The sample loses 0.5% of its weight on the TGA between room
temperature and 100 degrees C.
cis-Itraconazole:L-Tartaric Acid (Example 7)
PXRD: 4.13, 6.19, 8.49, 16.13, 17.23, 18.07, 19.13, 20.79, 22.85,
26.17
DSC: An endothermic transition at about 181 degrees C.
TGA: Less than 0.1% volatile components by weight by TGA
cis-Itraconazole:L-Malic acid (Example 8)
PXRD: 4.43, 6.07, 8.85, 15.93, 17.05, 20.49, 21.27, 22.85, 23.17,
26.17
DSC: The sample has a strong endothermic transition at about 154
degrees C.
TGA: The sample contained less than 0.1% volatile components by
weight
cis-ItraconazoleHCl:DL-Tartaric acid (Example 9)
PXRD: 3.73, 10.95, 13.83, 16.53, 17.75, 19.65, 21.11, 23.95
DSC: The sample has a peak endothermic transition at about 162
degrees C.
TGA: The sample contained less than 0.1% volatile components by
weight
Modafinil:Malonic acid (Example 10)
PXRD (Form I): 5.11, 9.35, 16.87, 18.33, 19.53, 21.38, 22.05, 22.89,
24.73, 25.19, 25.81, 28.59
PXRD (Form II): 5.90, 9.54, 15.79, 18.02, 20.01, 21.66, 22.47, 25.30
DSC (Form I): Endothermic transition at about 106 degrees C.
Raman (Form I): 1601, 1183, 1032, 1004, 814, 633, 265, 222
Modafinil:Glycolic acid (Example 11)
PXRD: 6.09, 9.51, 14.91, 15.97, 19.01, 20.03, 21.59, 22.43, 22.75,
23.75, 25.03, 25.71
Modafinil:Maleic acid (Example 12)
PXRD: 4.69, 6.15, 9.61, 10.23, 15.65, 16.53, 17.19, 18.01, 19.27,
19.53, 19.97, 21.83, 22.45, 25.65
5-fluorouracil:Urea (Example 13)
PXRD: 11.23, 12.69, 13.27, 15.93, 16.93, 20.37, 23.65, 25.55, 26.87,
32.49
DSC: Sharp endotherm at about 208 degrees C.
TGA: Approximately 32 percent weight loss between 150 and 220
degrees C.
Raman: 1347, 1024, 757, 644, 545
Hydrochlorothiazide:Nicotinic acid (Example 14)
PXRD: 8.57, 13.23, 14.31, 16.27, 17.89, 18.75, 21.13, 21.45, 24.41,
25.73, 26.57, 27.43
Hydrochlorothiazide:18-crown-6 (Example 15)
PXRD: 9.97, 10.43, 11.57, 11.81, 12.83, 14.53, 15.67, 16.61, 19.05,
20.31, 20.65, 21.09, 21.85, 22.45, 23.63, 24.21, 25.33, 26.73
Hydrochlorothiazide:Piperazine (Example 16)
PXRD: 6.85, 13.75, 15.93, 18.71, 20.67, 20.93, 23.27, 24.17, 28.33,
28.87, 30.89
Acetaminophen:4,4′-Bipyridine:water (Example 17)
DSC: Endothermic transition at about 58 degrees C.
Phenytoin:Pyridone (Example 18)
PXRD: 5.2, 11.1, 15.1, 16.2, 16.7, 17.8, 19.4, 19.8, 20.3, 21.2,
23.3, 26.1, 26.4, 27.3, 29.5
DSC: Endothermic transitions at about 233 and 271 degrees C.
TGA: 29.09 percent weight loss starting at about 193 degrees C.,
48.72 percent weight loss starting at about 238 degrees C., 18.38
percent weight loss starting at about 260 degrees C.
Aspirin:4,4′-Bipyridine (Example 19)
DSC: Endothermic transition at about 95 degrees C.
TGA: 9 percent weight loss starting at about 23 degrees C., 49.06
percent weight loss starting at about 103 degrees C., decomposition
starting at about 209 degrees C.
Ibuprofen:4,4′-Bipyridine (Example 20)
PXRD: 3.4, 6.9, 10.4, 17.3, 19.1
DSC: Endothermic transitions at about 65 and 119 degrees C.
TGA: 13.28 percent weight loss between room temperature and about
100 degrees C.
Flurbiprofen:4,4′-Bipyridine (Example 21)
PXRD: 16.8, 17.1, 18.1, 19.0, 20.0, 21.3, 22.7, 25.0, 26.0, 26.1,
28.2, 29.1
DSC: Endothermic transition at about 162 degrees C.
TGA: 30.93 percent weight loss starting at about 31 degrees C.,
46.26 percent weight loss starting at about 169 degrees C.
Flurbiprofen:trans-1,2-bis (4-pyridyl) ethylene (Example 22)
PXRD: 3.6, 17.3, 18.1, 18.4, 19.1, 22.3, 23.8, 25.9, 28.1
DSC: Endothermic transitions at about 100, 126, and 164 degrees C.
TGA: 91.79 percent weight loss starting at about 133 degrees C.
Carbamazepine:p-phthalaldehyde (Example 23)
PXRD: 8.5, 10.6, 11.9, 14.4, 15.1, 18.0, 18.5, 19.8, 23.7, 24.2,
26.4, 27.6, 27.8, 28.7, 29.3, 29.4
DSC: Endothermic transition at about 128 degrees C.
TGA: 17.66 percent weight loss starting at about 30 degrees C.,
17.57 percent weight loss starting at about 100 degrees C.
Carbamazepine:Nicotinamide (Example 24)
PXRD: 6.5, 8.8, 10.1, 13.2, 15.6, 17.7, 17.8, 18.3, 19.8, 20.4,
21.6, 22.6, 22.9, 26.4, 26.7, 28.0
DSC: Sharp endotherm at about 157 degrees C.
TGA: Decomposition beginning at about 150 degrees C.
Carbamazepine:Saccharin (Example 25)
PXRD: 6.9, 12.2, 13.6, 14.0, 14.1, 15.3, 15.9, 18.1, 18.7, 20.2,
21.3, 23.7, 26.3, 28.3
DSC: Endotherms were present at about 75 and 177 degrees C.
TGA: 3.342 percent weight loss starting at about 67 degrees C.,
55.09 percent weight loss starting at about 119 degrees C.
Carbamazepine:2,6-pyridinecarboxylic acid (Example 26)
TGA: 69 percent weight loss starting at about 215 degrees C.,
17 percent weight loss starting at about 392 degrees C.
Carbamazepine:5-nitroisophthalic acid (Example 27)
PXRD: 10.14, 15.29, 17.44, 21.17, 31.41, 32.65
DSC: Endotherm at about 191 degrees C.
TGA: 32.02 percent weight loss starting at about 202 degrees C.,
12.12 percent weight loss starting at about 224 degrees C., 17.94
percent weight loss starting at about 285 degrees C.
Carbamazepine:1,3,5,7-adamantane tetracarboxylic acid (Example 28)
TGA: 9 percent weight loss starting at about 189 degrees C., 52
percent weight loss starting at about 251 degrees C., 31 percent
weight loss starting at about 374 degrees C.
Carbamazepine:Benzoquinone (Example 29)
TGA: 20.62 percent weight loss starting at about 168 degrees C.,
78 percent weight loss starting at about 223 degrees C.
Carbamazepine:Trimesic acid (Example 30)
PXRD: 10.89, 12.23, 14.83, 16.25, 17.05, 18.13, 18.47, 21.47, 21.95,
24.57, 25.11, 27.99
DSC: Endothermic transition at about 273 degrees C.
TGA: 62.83 percent weight loss starting at about 253 degrees C.,
30.20 percent weight loss starting at about 278 degrees C.
All PXRD peaks are in units of degrees 2-theta
All Raman shifts are in units of cm⁻¹

### Example 31

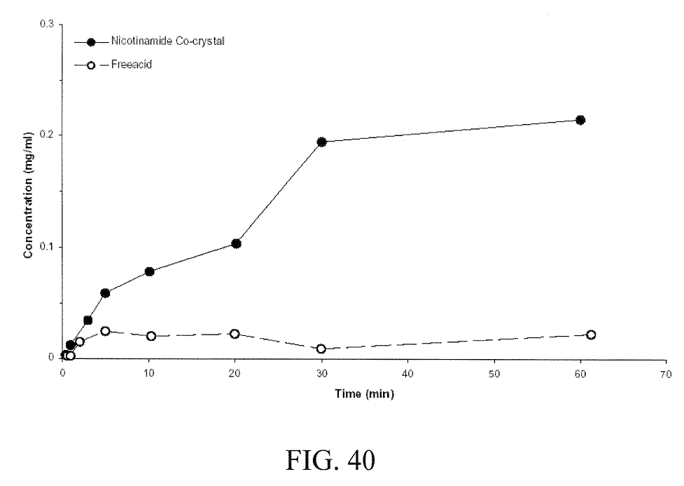

A co-crystal with a modulated dissolution profile has been prepared. Celecoxib: nicotinamide co-crystals were prepared via methods shown in Example 1. (See FIG. 40)

### Example 32

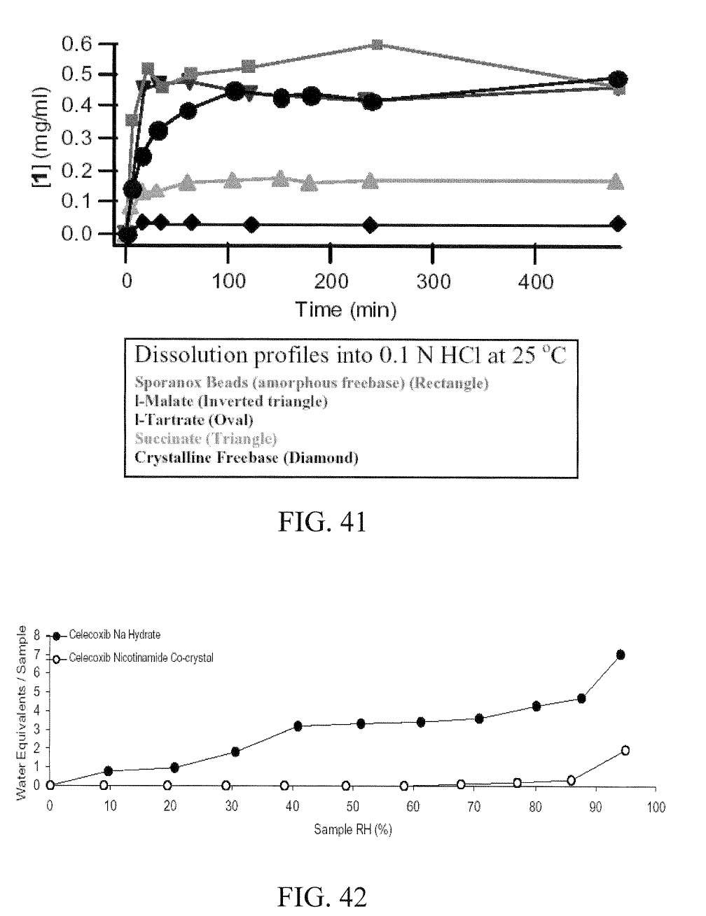

A co-crystal with a modulated dissolution profile has been prepared. cis-Itraconazole: succinic acid, cis-itraconazole:L-tartaric acid and cis-itraconazole:L-malic acid co-crystals were prepared via methods shown in Examples 5, 7 and 8. (See FIG. 41).

### Example 33

A co-crystal of an unsaltable or difficult to salt API has been prepared. Celecoxib: nicotinamide co-crystals were prepared via methods shown in Example 1.

### Example 34

A co-crystal with an improved hygroscopicity profile has been prepared. Celecoxib: nicotinamide co-crystals were prepared via methods shown in Example 1. (See FIG. 42).

### Example 35

A co-crystal with reduced form diversity as compared to the API has been prepared. Co-crystals of carbamazepine and saccharin have been prepared via method shown in Example 25.

### Example 36

The formulation of a modafinil:malonic acid form I co-crystal was completed using lactose. Two mixtures, one of modafinil and lactose, and the second of modafinil:malonic acid co-crystal and lactose, were ground together in a mortar an pestle. The mixtures targeted a 1:1 weight ratio of modafinil to lactose. In the modafinil and lactose mixture, 901.2 mg of modafinil and 901.6 mg of lactose were ground together. In the modafinil:malonic acid co-crystal and lactose mixture, 1221.6 mg of co-crystal and 871.4 mg of lactose were ground together. The resulting powders were analyzed by PXRD and DSC. The PXRD patterns and DSC thermograms of the mixtures showed virtually no change upon comparison with both individual components. The DSC of the co-crystal mixture showed only the co-crystal melting peak at 113.6 degrees C. with a heat of fusion of 75.9 J/g. This heat of fusion is 59.5% of that found for the co-crystal alone (127.5 J/g). This result is consistent with a 58.4% weight ratio of co-crystal in the mixture. The DSC of the modafinil and lactose mixture had a melting point of 165.7 degrees C. This is slightly lower then the measured melting point of modafinil (168.7 degrees C.). The heat of fusion of the mixture (59.3 J/g) is 46.9% that of the modafinil alone (126.6 J/g), which is consistent with the estimated value of 50%.

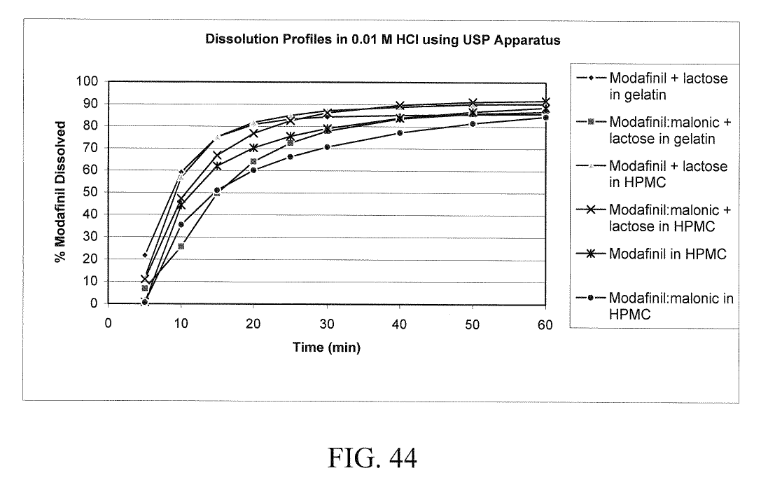

The in vitro dissolution of both the modafinil:malonic acid form I co-crystal and pure modafinil were tested in capsules. Both gelatin and hydroxypropylmethyl cellulose (HPMC) capsules were used in the dissolution study. The capsules were formulated with and without lactose. All formulations were ground in a mortar and pestle prior to transfer into a capsule. The dissolution of the capsules was tested in 0.01 M HCl (See FIG. 44).

### In 0.01M HCl, Using Sieved and Ground Materials in Gelatin Capsules:

Modafinil and the modafinil:malonic acid form I co-crystal were passed through a 38 micrometer sieve. Gelatin capsules (Size 0, B&B Pharmaceuticals, Lot #15-01202) were filled with 200.0 mg sieved modafinil, 280.4 mg sieved modafinil:malonic acid co-crystal, 200.2 mg ground modafinil, or 280.3 mg ground modafinil:malonic acid co-crystal. Dissolution studies were performed in a Vankel VK 7000 Benchsaver Dissolution Testing Apparatus with the VK750D heater/circulator set at 37 degrees C. At 0 minutes, the capsules were dropped into vessels containing 900 mL 0.01 M HCl and stirred by paddles.

Absorbance readings were taken using a Cary 50 Spectrophotometer (wavelength set at 260 nm) at the following time points: 0, 5, 10, 15, 20, 25, 30, 40, 50, and 60 minutes. The absorbance values were compared to those of standards and the modafinil concentrations of the solutions were calculated.

In 0.01M HCl, Using Ground Materials in Gelatin or HPMC Capsules, with and without Lactose:

Modafinil and the modafinil:malonic acid form I co-crystal were mixed with equivalent amounts of lactose (Spectrum, Lot QV0460) for approximately 5 minutes. Gelatin capsules (Size 0, B&B Pharmaceuticals, Lot #15-01202) were filled with 400.2 mg modafinil and lactose (approximately 200 mg modafinil), or 561.0 mg modafinil:malonic acid form I co-crystal and lactose (approximately 200 mg modafinil). HPMC capsules (Size O, Shionogi, Lot #A312A6) were filled with 399.9 mg modafinil and lactose, 560.9 mg modafinil:malonic acid co-crystal and lactose, 199.9 mg modafinil, or 280.5 mg modafinil:malonic acid form I co-crystal. The dissolution study was carried out as described above.

### Example 37

The modafinil:malonic acid form I co-crystal (from Example 10) was administered to dogs in a pharmacokinetic study. Particles of modafinil:malonic acid co-crystal with a median particle size of about 16 micrometers were administered in the study. As a reference, micronized modafinil with a median particle size of about 2 micrometers was also administered in the study. The AUC of the modafinil:malonic acid co-crystal was determined to be 40 to 60 percent higher than that of the pure modafinil. Such a higher bioavailability illustrates the modulation of an important pharmacokinetic parameter due to an embodiment of the present invention. A compilation of important pharmacokinetic parameters measured during the animal study are included in Table XXV.

TABLE XXV
Pharmacokinetic parameters of modafinil:malonic
acid co-crystal and pure modafinil in dogs

| Parameter | Pure Modafinil | Modafinil:malonic acid co-crystal |
|---|---|---|
| Median particle size | 2 micrometers | 16 micrometers |
| Cₘₐₓ (ng/mL) | 11.0 ± 5.9 | 10.3 ± 3.4 |
| Tₘₐₓ (hours) | 1.3 ± 0.6 | 1.7 ± 0.6 |
| AUC (relative) | 1.0 | 1.4-1.6 |
| Half-life (hours) | 2.1 ± 0.7 | 5.1 ± 2.4 |

The increased half-life and bioavailability of modafinil in the malonic acid form I co-crystal may be due to the presence of malonic acid. It is believed that the malonic acid may be inhibiting one or more pathways responsible for the metabolism or elimination of modafinil. It is noted that modafinil and malonic acid share a similar structure: each including two carbonyl or sulfonyl groups separated by a —CH₂— and each molecule is terminated with a group that is capable of participation in a hydrogen bond with an enzyme. Such a mechanism may take place with other APIs or co-crystal formers of similar structure.

### Example 38

The stability of the modafinil:malonic acid form I co-crystal was measured at various temperatures and relative humidities over a four week period. No degradation was found to occur at 20 or 40 degrees C. At 60 degrees C., about 0.14 percent degradation per day was determined based on a simple exponential model. At 80 degrees C., about 8 percent degradation per day was determined.

Lengthy table referenced here
US20100311701A1-20101209-T00001
Please refer to the end of the specification for access instructions.

Lengthy table referenced here
US20100311701A1-20101209-T00002
Please refer to the end of the specification for access instructions.

Lengthy table referenced here
US20100311701A1-20101209-T00003
Please refer to the end of the specification for access instructions.

Lengthy table referenced here
US20100311701A1-20101209-T00004
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20100311701A1). An electronic copy of the table will also be available from the USPTO upon request and payment of the fee set forth in 37 CFR 1.19(b)(3).

We claim:

1. A process for preparing a pharmaceutical co-crystal composition comprising an API and a co-crystal former, comprising:
(a) providing an API and a co-crystal former, wherein the API is a liquid or a solid at room temperature and the co-crystal former is a solid at room temperature;
(b) grinding, heating, co-subliming, co-melting, or contacting in solution the API with the co-crystal former under crystallization conditions, so as to form a solid phase, wherein the API and co-crystal former are hydrogen bonded to each other;
(c) isolating co-crystals formed thereby; and
(d) incorporating the co-crystals into a pharmaceutical composition.

2. The process of claim 1, wherein:
(a) the co-crystal former is selected from a co-crystal former of Table I or Table II;
(b) the API is selected from an API of Table IV;
(c) the API is selected from an API of Table IV and the co-crystal former is selected from a co-crystal former of Table I or Table II;
(d) the API is a liquid at room temperature;
(e) the API is a solid at room temperature;
(f) the API has at least one functional group selected from the group consisting of: ether, thioether, alcohol, thiol, aldehyde, ketone, thioketone, nitrate ester, phosphate ester, thiophosphate ester, ester, thioester, sulfate ester, carboxylic acid, phosphonic acid, phosphinic acid, sulfonic acid, amide, primary amine, secondary amine, ammonia, tertiary amine, imine, thiocyanate, cyanamide, oxime, nitrile, diazo, organohalide, nitro, S-heterocyclic ring, thiophene, N-heterocyclic ring, pyrrole, O-heterocyclic ring, furan, epoxide, peroxide, hydroxamic acid, imidazole, and pyridine;
(g) the co-crystal former has at least one functional group selected from the group consisting of: ether, thioether, alcohol, thiol, aldehyde, ketone, thioketone, nitrate ester, phosphate ester, thiophosphate ester, ester, thioester, sulfate ester, carboxylic acid, phosphonic acid, phosphinic acid, sulfonic acid, amide, primary amine, secondary amine, ammonia, tertiary amine, imine, thiocyanate, cyanamide, oxime, nitrile, diazo, organohalide, nitro, S-heterocyclic ring, thiophene, N-heterocyclic ring, pyrrole, O-heterocyclic ring, furan, epoxide, peroxide, hydroxamic acid, imidazole, and pyridine; or
(h) the difference in pKₐ between the API and the co-crystal former does not exceed 2.

3. A process for preparing a pharmaceutical co-crystal composition comprising an API, a co-crystal former, and a third molecule, comprising:
(a) providing an API and a co-crystal former, wherein the API is a liquid or a solid at room temperature and the co-crystal former is a solid at room temperature;
(b) grinding, heating, co-subliming, co-melting, or contacting in solution the API with the co-crystal former under crystallization conditions, so as to form a solid phase, wherein the API and the third molecule are bonded to each other, and further wherein the co-crystal former and the third molecule are hydrogen bonded to each other;
(c) isolating co-crystals formed thereby; and
(d) incorporating the co-crystals into a pharmaceutical composition.

4. The process of claim 3, wherein:
(a) the co-crystal former is selected from a co-crystal former of Table I or Table II;
(b) the API is selected from an API of Table IV;
(c) the API is selected from an API of Table IV and the co-crystal former is selected from a co-crystal former of Table I or Table II;
(d) the API is a liquid at room temperature;
(e) the API is a solid at room temperature;
(f) the API has at least one functional group selected from the group consisting of: ether, thioether, alcohol, thiol, aldehyde, ketone, thioketone, nitrate ester, phosphate ester, thiophosphate ester, ester, thioester, sulfate ester, carboxylic acid, phosphonic acid, phosphinic acid, sulfonic acid, amide, primary amine, secondary amine, ammonia, tertiary amine, imine, thiocyanate, cyanamide, oxime, nitrile, diazo, organohalide, nitro, S-heterocyclic ring, thiophene, N-heterocyclic ring, pyrrole, O-heterocyclic ring, furan, epoxide, peroxide, hydroxamic acid, imidazole, and pyridine;
(g) the co-crystal former has at least one functional group selected from the group consisting of: ether, thioether, alcohol, thiol, aldehyde, ketone, thioketone, nitrate ester, phosphate ester, thiophosphate ester, ester, thioester, sulfate ester, carboxylic acid, phosphonic acid, phosphinic acid, sulfonic acid, amide, primary amine, secondary amine, ammonia, tertiary amine, imine, thiocyanate, cyanamide, oxime, nitrile, diazo, organohalide, nitro, S-heterocyclic ring, thiophene, N-heterocyclic ring, pyrrole, O-heterocyclic ring, furan, epoxide, peroxide, hydroxamic acid, imidazole, and pyridine; or
(h) the difference in pKₐ between the API and the co-crystal former does not exceed 2.

5. A process for preparing a pharmaceutical co-crystal composition comprising a first and a second API, comprising:
(a) providing a first and a second API, wherein each API is either a liquid or a solid at room temperature;
(b) grinding, heating, co-subliming, co-melting, or contacting in solution the APIs under crystallization conditions, so as to form a solid phase, wherein the APIs are hydrogen bonded to a molecule;
(c) isolating co-crystals formed thereby; and
(d) incorporating the co-crystals into a pharmaceutical composition.

6. The process of claim 5, wherein:
(a) the first API is hydrogen bonded to the second API;
(b) an API is selected from an API of Table IV;
(c) each API is selected from an API of Table IV;
(d) an API is a liquid at room temperature and the other API is a solid at room temperature;
(e) each API is a solid at room temperature;
(f) an API has at least one functional group selected from the group consisting of: ether, thioether, alcohol, thiol, aldehyde, ketone, thioketone, nitrate ester, phosphate ester, thiophosphate ester, ester, thioester, sulfate ester, carboxylic acid, phosphonic acid, phosphinic acid, sulfonic acid, amide, primary amine, secondary amine, ammonia, tertiary amine, imine, thiocyanate, cyanamide, oxime, nitrile, diazo, organohalide, nitro, S-heterocyclic ring, thiophene, N-heterocyclic ring, pyrrole, O-heterocyclic ring, furan, epoxide, peroxide, hydroxamic acid, imidazole, and pyridine;
(g) each API has at least one functional group selected from the group consisting of: ether, thioether, alcohol, thiol, aldehyde, ketone, thioketone, nitrate ester, phosphate ester, thiophosphate ester, ester, thioester, sulfate ester, carboxylic acid, phosphonic acid, phosphinic acid, sulfonic acid, amide, primary amine, secondary amine, ammonia, tertiary amine, imine, thiocyanate, cyanamide, oxime, nitrile, diazo, organohalide, nitro, S-heterocyclic ring, thiophene, N-heterocyclic ring, pyrrole, O-heterocyclic ring, furan, epoxide, peroxide, hydroxamic acid, imidazole, and pyridine; or
(h) the difference in pKₐ between the first API and the second API does not exceed 2.
